(19) United States
(12) Patent Application Publication
(10) Pub. No.: US 2026/0297102 A1
(43) Pub. Date: Oct. 1, 2026

(54) HETEROCYCLIC COMPOUNDS AS PARP1 INHIBITORS

(71) Applicant: Synnovation Therapeutics, Inc., Wilmington, DE (US)

(72) Inventors: Jun Pan, Media, PA (US); Liangxing Wu, Wilmington, DE (US); Wenqing Yao, Chadds Ford, PA (US)

(21) Appl. No.: 19/479,040

(22) PCT Filed: Apr. 26, 2024
(86) PCT No.: PCT/US2024/026578

§ 371 Date: Oct. 27, 2025

Related U.S. Application Data
(60) Provisional application No. 63/462,569, filed on Apr. 28, 2023.
(60) Provisional application No. 63/521,382, filed on Jun. 16, 2023.
(60) Provisional application No. 63/509,294, filed on Jun. 21, 2023.

(51) Int. Cl. C07D 487/16; A61K 31/519; A61K 31/5383; C07D 471/16; C07D 487/06; C07D 498/16

(52) U.S. Cl. CPC C07D 487/16; A61K 31/519; A61K 31/5383; C07D 471/16; C07D 487/06; C07D 498/16

(57) ABSTRACT

The present disclosure provides compounds, compositions, and methods useful for inhibiting PARP1, and/or treating a disease, disorder, or condition associated with PARP1, such as cancer.

### TECHNICAL FIELD

The present disclosure provides heterocyclic compounds as well as their pharmaceutical compositions that modulate the activity of PARP1 and are useful in the treatment of various diseases related to PARP1, including cancer.

### BACKGROUND

Poly ADP-Ribose Polymerases (PARPs) are a superfamily of enzymes that comprise at least 17 family members. Some of these PARP enzymes, including PARP1, PARP2, PARP5A, and PARP5B, catalyze NAD+ substrate to covalently attach poly ADP-ribose (PAR), a linear or branched, heterogeneous polymer to acceptor proteins, while other members attach mono ADP-ribose (MAR) to acceptor proteins. Accumulating evidence suggests that PARP enzymes have distinct functions. Among those identified PARPs, PARP1, PARP2 and PARP3 are DNA-dependent of which enzymatic activity is strongly stimulated by endogenous and exogenous DNA damage (van Beek, L. et al. Int. J. Mol. Sci., 2021, 22, 5112). These first three PARP enzyme members are therefore important for the regulation of DNA damage repair through a mechanism called Poly ADP-ribosylation (PARylation).

PARylation is a dynamic, short-lived post-translational modification, which can take place in very few minutes. The polymer generated by PARylation can then be degraded through another enzyme called poly ADP-ribose glycohydrolase (PARG). These key enzymes protect cells from DNA damage-induced cell dysfunction and cell death. Because of the large size and highly charged property of PAR, PARylation dramatically alters the regulation of DNA damage response (DDR), cellular stress response and RNA transcription/processing in various biological systems (Feng, X. et al. Int. Rev. Cell Mol. Biol., 2013, 304, 227; Kraus, W. L., Mol. Cell, 2015, 58, 902; Cohen, M. S., et al. Nat. Chem. Biol., 2018, 14, 236).

PARP1, the founding member of the PARP superfamily, contributing to over 90% of PARylation, has been extensively studied for its pivotal role in DNA damage response, especially for the repair of DNA single strand breaks (SSBs) (Durkacz, B. W., et al. Nature, 1980, 283, 593). The basal level of PARylation in quiescent cells is typically below detection. When exposed to genotoxic stress, PARP1 is rapidly activated by self-modification (auto-PARylation), which initiates the DNA damage-response signaling pathways. This process includes a complex cascade of signaling events starting from binding of PARP proteins to the damage sites, to PARylating and recruiting of repair factors, and eventually dissociating from the damage sites (Bai, P., Mol. Cell, 2015, 58, 947). PARP2 is involved in DNA damage repair as well. However, distinct from PARP1, mounting evidence suggests that PARP2 also plays crucial roles in the development and maintenance of hematopoietic cells and some other tissues.

Clinical data have clearly demonstrated the effectiveness of PARP inhibitors in treating a variety of human cancers, particularly the BRCA1/2-mutated, homologous recombination deficient (HRD) cancers. PARP inhibition compromises repair of SSBs by blocking PARylation. On the other hand, PARP inhibitors also trap the PARP protein onto DNA damage sites. PARP trapping leads to blockade of DNA replication, resulting in single-ended DNA double strand breaks (DSBs) due to collapse of replication forks. These breaks require homologous recombination (HR) for faithful repair. Otherwise, these cells would die due to accumulated DNA damage and genomic instability. PARP hyperactivation is frequently observed in cancer patients with HRD tumors. This correlation clearly indicates a hyper-reliance of these tumors on PARP mediated DNA repair pathways (Helleday, T., Mol. Oncol., 2011, 5, 387). These mechanistic studies provide the rationale for targeting HRD cancers with PARP inhibitors.

Although PARP1 is the primary target for developing PARP inhibitors, most if not all current PARP inhibitors also suppress enzymatic activities of other PARPs, particularly PARP2, a close paralog of PARP1 that sharing a 69% identity of its catalytic domain. PARP2 catalyzes only about 10% of cellular PARylation in the presence of PARP1 (Ame, J. C., et al. Bioessays, 2004, 26, 882; Ame, J. C., et al. J. Biol. Chem., 1999, 274, 17860). Despite the functional redundancy with PARP1, PARP2 also has its own unique functions in controlling hematopoiesis, spermatogenesis, adipogenesis and transcriptional regulation. Therefore, pharmacologic inhibition of the PARP2 enzyme may lead to unfavorable effects in aforementioned tissues, consequently resulting in adverse effects in clinical applications (Farres, J., et al. Blood, 2013, 122, 44; Chen, Q., et al. Nat. Commun., 2018, 9, 3233; Gui, B., et al. PNAS, 2019, 116, 14573). Taken together, selective inhibition of PARP1 while retaining the essential functions of PARP2 and other PARP family members is expected to maximize efficacy of PARP inhibitors in treating human cancers while minimizing its unfavorable side effects.

### SUMMARY

The present disclosure provides, inter alia, compounds of Formula I.

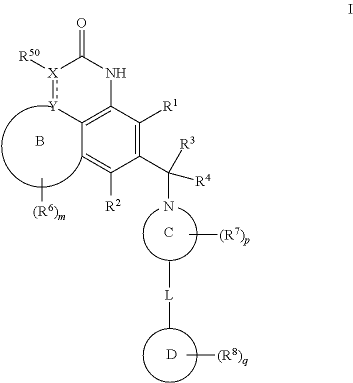

or pharmaceutically acceptable salts thereof, wherein constituent members are defined herein.

The present disclosure further provides a pharmaceutical composition comprising a compound of the disclosure, or a pharmaceutically acceptable salt thereof, and a pharmaceutically acceptable carrier or excipient.

The present disclosure further provides methods of inhibiting PARP1 activity, comprising contacting the PARP1 with a compound described herein, or a pharmaceutically acceptable salt thereof.

The present disclosure further provides methods of treating a disease or a disorder associated with PARP1 in a patient by administering to the patient a therapeutically effective amount of a compound of the disclosure, or a pharmaceutically acceptable salt thereof.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

### DETAILED DESCRIPTION

The present application provides a compound of Formula I:

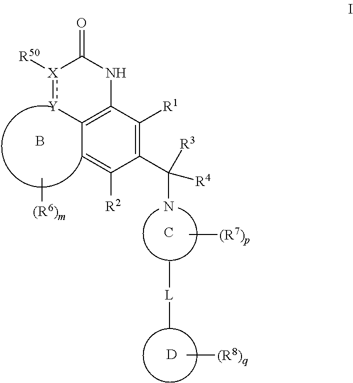

or a pharmaceutically acceptable salt thereof, wherein:
X is C or N;
Y is C or N;
[symbol] is a single or double bond;
m is 0, 1, 2, 3, 4, 5, or 6;
p is 0, 1, 2, 3, 4, 5, or 6;
q is 0, 1, 2, 3, 4, 5, or 6;
Ring B is C₅₋₁₀ cycloalkyl, phenyl, 5-10 membered heterocycloalkyl, or 5-6 membered heteroaryl;
Ring C is 4-14 membered heterocycloalkyl;
Ring D is C₃₋₁₀ cycloalkyl, C₆₋₁₀ aryl, 4-10 membered heterocycloalkyl, or 5-10 membered heteroaryl;
L is selected from bond, C₁₋₄ alkylene, C₁₋₄ haloalkylene, C₃₋₇ cycloalkylene, 4-7 membered heterocycloalkylene, —C₃₋₇ cycloalkylene-C₁₋₄ alkyl-, -(4-7 membered heterocycloalkylene)-C₁₋₄ alkyl-, —O—, —N(R^{L})—, —C(O)—, —C(O)N(R^{L})—, —N(R^{L})C(O)N(R^{L})—, —N(R^{L})C(O)O—, —S(O)₂—, —N(R^{L})S(O)₂—, and —N(R^{L})S(O)₂N(R^{L})—, wherein the C₁₋₄ alkylene, C₁₋₄ haloalkylene, C₃₋₇ cycloalkylene, 4-7 membered heterocycloalkylene, C₃₋₇ cycloalkylene-C₁₋₄ alkyl, and (4-7 membered heterocycloalkylene)-C₁₋₄ alkyl of L are each optionally substituted with 1, 2, 3, or 4 independently selected R^{G} substituents;
each R^{L} is independently selected from H, C₁₋₆ alkyl, and C₁₋₆ haloalkyl;
R¹ is selected from H, halo, CN, C₁₋₃ alkyl, C₁₋₃ haloalkyl, C₁₋₃ alkoxy, and C₁₋₃ haloalkoxy;
R² is selected from H, halo, CN, C₁₋₃ alkyl, C₁₋₃ haloalkyl, C₁₋₃ alkoxy, and C₁₋₃ haloalkoxy;
R³ is selected from H, halo, C₁₋₃ alkyl, and C₁₋₃ haloalkyl;
R⁴ is selected from H, halo, C₁₋₃ alkyl, and C₁₋₃ haloalkyl;
or, R³ and R⁴, together with the carbon atom to which they are attached, form a C₃₋₇ cycloalkyl, or 4-7 membered heterocycloalkyl group, wherein the C₃₋₇ cycloalkyl, and 4-7 membered heterocycloalkyl group is optionally substituted with 1, 2, 3, 4, 5, or 6 independently selected R^{G} substituents;
R⁵⁰ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a50}, —SR^{a50}, —NR^{c50}R^{d50}, —NO₂, —C(O)R^{a50}, —C(O)OR^{a50}, —C(O)NR^{c50}R^{d50}, —C(O)NR^{c50}(OR^{a50}), —OC(O)R^{a50}, —OC(O)NR^{c50}R^{d50}, —OC(O)OR^{a50}, —OS(O)₂R^{b50}, —OS(O)₂NR^{c50}R^{d50}, —NR^{c50}C(O)R^{a50}, —NR^{c50}C(O)OR^{a50}, —NR^{c50}C(O)NR^{c50}R^{d50}, —NR^{c50}S(O)₂R^{b50}, —NR^{c50}S(O)₂NR^{c50}R^{d50}, —NR^{c50}OR^{a50}, —NR^{c50}S(O)R^{b50}, —NR^{c50}S(O)NR^{c50}R^{d50}, —S(O)R^{b50}, —S(O)₂R^{b50}, —S(O)NR^{c50}R^{d50}, —S(O)₂NR^{c50}R^{d50}, —C(═NR^{e50})R^{a50}, —C(═NR^{e50})NR^{c50}R^{d50}, —NR^{c50}C(═NR^{e50})R^{a50}, —NR^{c50}C(═NR^{e50})NR^{c50}R^{d50}, —NR^{c50}S(O)(═NR^{e50})R^{b50}, —NR^{c50}S(O)(═NR^{e50})NR^{c50}R^{d50}, —OS(O)(═NR^{e50})R^{b50}, —S(O)(═NR^{e50})R^{b50}, —S(O)(═NR^{e50})NR^{c50}R^{d50}, —C(O)NR^{c50}S(O)₂R^{b50}, —C(O)NR^{c50}S(O)₂NR^{c50}R^{d50}, —S(O)₂NR^{c50}C(O)R^{b50}, —NR^{c50}S(O)NR^{c50}C(O)R^{b50}, and —P(O)R^{f50}R^{g50}, wherein the C₁₋₆ alkyl, C₂₋₆ alkenyl, C₂-6 alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁵⁰ are each optionally substituted with 1, 2, 3, 4, 5, or 6 independently selected R^{G} substituents;
each R^{a50}, R^{c50}, and R^{d50} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a50}, R^{c50} and R^{d50} are each optionally substituted with 1, 2, 3, 4, 5, or 6 independently selected R^{G} substituents;
or, any R^{c50} and R^{d50} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b50} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b50} are each optionally substituted with 1, 2, 3, 4, 5, or 6 independently selected R^{G} substituents;
each R^{e50} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f50} and R^{g50} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
or, R⁵⁰ and one R⁶, taken together with the atoms to which they are attached, form a Ring A which is selected from C₅₋₁₀ cycloalkyl, phenyl, 5-10 membered heterocycloalkyl, and 5-6 membered heteroaryl, wherein the C₅₋₁₀ cycloalkyl, phenyl, 5-10 membered heterocycloalkyl, and 5-6 membered heteroaryl group is optionally substituted with 1, 2, 3, 4, 5, or 6 independently selected R⁵ substituents;
each R⁵ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{a5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R^{b5}, —NR^{c5}S(O)₂NR^{c5}R^{d5}, —NR^{c5}OR^{a5}, —NR^{c5}S(O)R^{b5}, —NR^{c5}S(O)NR^{c5}R^{d5}, —S(O)R^{b5}, —S(O)₂R^{b5}, —S(O)NR^{c5}R^{d5}, —S(O)₂NR^{c5}R^{d5}, —C(═NR^{e5})R^{a5}, —C(═NR^{e5})NR^{c5}R^{d5}, —NR^{c5}C(═NR^{e5})R^{a5}, —NR^{c5}C(═R^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g5}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁵ are each optionally substituted with 1, 2, 3, 4, 5, or 6 independently selected R^{G} substituents;
each R^{a5}, R^{c5} and R^{d5} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a5}, R^{c5}, and R^{d5} are each optionally substituted with 1, 2, 3, 4, 5, or 6 independently selected R^{G} substituents;
or, any R^{a5} and R^{d5} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b5} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b5} are each optionally substituted with 1, 2, 3, 4, 5, or 6 independently selected R^{G} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f5} and R^{g5} are independently selected from H, C₁₋₆ alkyl, C₁₋₆ alkoxy, C₁. 6 haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁶ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6}, —SR^{a6}, —NR^{c6}R^{d6}, —NO₂, —C(O)R^{a6}, —C(O)OR^{a6}, —C(O)NR^{c6}R^{d6}, —C(O)NR^{c6}(OR^{a6}), —OC(O)R^{a6}, —OC(O)NR^{c6}R^{d6}, —OC(O)OR^{a6}, —OS(O)₂R^{b6}, —OS(O)₂NR^{c6}R^{d6}, —NR^{c6}C(O)R^{a6}, —NR^{c6}C(O)OR^{a6}, —NR^{c6}C(O)NR^{c6}R^{d6}, —NR^{c6}S(O)₂R^{b6}, —NR^{c6}S(O)₂NR^{c6}R^{d6}, —NR^{c6}OR^{a6}, —NR^{c6}S(O)R^{b6}, —NR^{c6}S(O)NR^{c6}R^{d6}, —S(O)R^{b6}, —S(O)₂R^{b6}, —S(O)NR^{c6}R^{d6}, —S(O)₂NR^{c6}R^{d6}, —C(═NR^{e6})R^{a6}, —C(═NR^{e6})R^{c6}R^{d6}, —R^{c6}C(═NR^{e6})R^{a6}, —NR^{c6}C(═NR^{e6})NR^{c6}R^{d6}, —NR^{c6}S(O)(═NR^{e6})R^{b6}, —NR^{c6}S(O)(═NR^{e6})NR^{c6}R^{d6}, —OS(O)(═NR^{e6})R^{b6}, —S(O)(═NR^{e6})R^{b6}, —S(O)(═NR^{e6})NR^{c6}R^{d6}, —C(O)NR^{c6}S(O)₂R^{b6}, —C(O)NR^{c6}S(O)₂NR^{c6}R^{d6}, —S(O)₂NR^{c6}C(O)R^{b6}, —NR^{c6}S(O)NR^{c6}C(O)R^{b6}, and —P(O)R^{f6}R^{g6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, 4, 5, or 6 independently selected R^{G} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a6}, R^{c6}, and R^{d6} are each optionally substituted with 1, 2, 3, 4, 5, or 6 independently selected R^{G} substituents;
or, any R^{c6} and R^{d6} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b6} are each optionally substituted with 1, 2, 3, 4, 5, or 6 independently selected R^{G} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f6} and R^{g6} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁷ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a7}, —SR^{a7}, —NR^{c7}R^{d7}, —NO₂, —C(O)R^{a7}, —C(O)OR^{a7}, —C(O)NR^{c7}R^{d7}, —C(O)NR^{c7}(OR^{a7}), —OC(O)R^{a7}, —OC(O)NR^{c7}R^{d7}, —OC(O)OR^{a7}, —OS(O)₂R^{b7}, —OS(O)₂NR^{c7}R^{d7}, —NR^{c7}C(O)R^{a7}, —NR^{c7}C(O)OR^{a7}, —NR^{c7}C(O)NR^{c7}R^{d7}, —NR^{c7}S(O)₂R^{b7}, —NR^{c7}S(O)₂NR^{c7}R^{d7}, —NR^{c7}OR^{a7}, —NR^{c7}S(O)R^{b7}, —R^{c7}S(O)NR^{c7}R^{d7}, —S(O)R^{b7}, —S(O)₂R^{b7}, —S(O)NR^{c7}R^{d7}, —S(O)₂NR^{c7}R^{d7}, —C(═NR^{e7})R^{a7}, —C(═NR^{e7})NR^{c7}R^{d7}, —NR^{c7}C(═NR^{e7})R^{a7}, —NR^{c7}C(═NR^{c7})NR^{c7}R^{d7}, —NR^{c7}S(O)(═NR^{e7})R^{b7}, —NR^{c7}S(O)(═NR^{e7})NR^{c7}R^{d7}, —OS(O)(═NR^{e7})R^{b7}, —S(O)(═NR^{e7})R^{b7}, —S(O)(═NR^{e7})NR^{c7}R^{d7}, —C(O)NR^{c7}S(O)₂R^{b7}, —C(O)NR^{c7}S(O)₂NR^{c7}R^{d7}, —S(O)₂NR^{c7}C(O)R^{b7}, —NR^{c7}S(O)NR^{c7}C(O)R^{b7} and —P(O)R^{f7}R^{g7}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁷ are each optionally substituted with 1, 2, 3, 4, 5, or 6 independently selected R^{G} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a7}, R^{c7}, and R^{d7} are each optionally substituted with 1, 2, 3, 4, 5, or 6 independently selected R^{G} substituents;
or, any R^{c7} and R^{d7} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b7} are each optionally substituted with 1, 2, 3, 4, 5, or 6 independently selected R^{G} substituents;
each R^{c7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁸ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, —C(═NR^{e8})R^{a8}, —C(═NR^{e8})NR^{c8}R^{d8}, —NR^{c8}C(═NR^{e8})R^{a8}, —NR^{c8}C(═NR^{e8})NR^{c8}R^{d8}, —NR^{c8}S(O)(═NR^{e8})R^{b8}, —NR^{c8}S(O)(═NR^{e8})NR^{c8}R^{d8}, —OS(O)(═NR^{e8})R^{b8}, —S(O)(═NR^{e8})R^{b8}, —S(O)(═NR^{e8})NR^{c8}R^{d8}, —C(O)NR^{c8}S(O)₂R^{b8}, —C(O)NR^{c8}S(O)₂NR^{c8}R^{d8}, —S(O)₂NR^{c8}C(O)R^{b8}, —NR^{c8}S(O)NR^{c8}C(O)R^{b8}, and —P(O)R^{f8}R^{g8}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁸ are each optionally substituted with 1, 2, 3, 4, 5, or 6 independently selected R^{8A} substituents;
or, a R⁷ and a R⁸, together with the atoms to which they are attached, form a C₅₋₁₀ cycloalkyl, phenyl, 5-10 membered heterocycloalkyl, or 5-6 membered heteroaryl group, wherein the C₅₋₁₀ cycloalkyl, phenyl, 5-10 membered heterocycloalkyl, or 5-6 membered heteroaryl group is optionally substituted with 1, 2, 3, 4, 5, or 6 independently selected R^{8A} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{c8}, and R^{d8} are each optionally substituted with 1, 2, 3, 4, 5, or 6 independently selected R^{8A} substituents;
or, any R^{c8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{b8} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8} are each optionally substituted with 1, 2, 3, 4, 5, or 6 independently selected R^{8A} substituents;
each R⁸ is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{c8A}R^{d8A}, —NO₂, —C(O)R^{a8A}, —C(O)OR^{a8A}, —C(O)NR^{c8A}R^{d8A}, —C(O)NR^{c8A}(OR^{a8A}), —OC(O)R^{a8A}, —OC(O)NR^{c8A}R^{d8A}, —OC(O)OR^{a8A}, —OS(O)₂R^{b8A}, —OS(O)₂NR^{c8A}R^{d8A}, —NR^{c8A}C(O)R^{a8A}, —NR^{c8A}C(O)OR^{a8A}, —NR^{c8A}C(O)NR^{c8A}R^{d8A}, —NR^{e8A}S(O)₂R^{b8A}, —NR^{c8A}S(O)₂NR^{c8A}R^{d8A}, —NR^{e8A}OR^{a8A}, —NR^{c8A}S(O)R^{b8A}, —NR^{e8A}S(O)NR^{c8A}R^{d8A}, —S(O)R^{b8A}, —S(O)₂R^{b8A}, —S(O)NR^{c8A}R^{d8A}, —S(O)₂NR^{c8A}R^{d8A}, —C(═NR^{c8A})R^{b8A}, —C(═NR^{c8A})NR^{c8A}R^{d8A}, —NR^{c8A}C(═NR^{c8A})R^{b8A}, —NR^{c8A}C(═NR^{c8A})NR^{c8A}R^{d8A}, —NR^{e8A}S(O)(═NR^{c8A})R^{b8A}, —NR^{e8A}S(O)(═NR^{c8A})NR^{c8A}R^{d8A}, —OS(O)(═NR^{c8A})R^{b8A}, —S(O)(═NR^{c8A})R^{b8A}, —S(O)(═NR^{c8A})NR^{c8A}R^{d8A}, —C(O)NR^{c8A}S(O)₂R^{b8A}, —C(O)NR^{c8A}S(O)₂NR^{c8A}R^{d8A}, —S(O)₂NR^{c8A}C(O)R^{b8A}, —NR^{e8A}S(O)NR^{c8A}C(O)R^{b8A}, and —P(O)R^{f8A}R^{g8A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{8A} are each optionally substituted with 1, 2, 3, 4, 5, or 6 independently selected R^{G} substituents;
each R^{a8A}, R^{c8A}, and R^{d8A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8A}, R^{c8A} and R^{d8A} are each optionally substituted with 1, 2, 3, 4, 5, or 6 independently selected R^{G} substituents;
or, any R^{c8A} and R^{d8A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b8A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8A} are each optionally substituted with 1, 2, 3, 4, 5, or 6 independently selected R^{G} substituents;
each R^{e8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, R⁵⁰ is selected from H, halo, C₁₋₆ alkyl, C₂₋₆ alkenyl, C₂-6 alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a50}, —SR^{a50}, —NR^{c50}R^{d50}, —NO₂, —C(O)R^{a50}, —C(O)OR^{a50}, —C(O)NR^{c50}R^{d50}, —C(O)NR^{c50}(OR^{a50}), —OC(O)R^{a50}, —OC(O)NR^{c50}R^{d50}, —OC(O)OR^{a50}, —OS(O)₂R^{b50}, —OS(O)₂NR^{c50}R^{d50}, —NR^{c50}C(O)R^{a50}, —NR^{c50}C(O)OR^{a50}, —NR^{c50}C(O)NR^{c50}R^{d50}, —NR^{c50}S(O)₂R^{b50}, —NR^{c50}S(O)₂NR^{c50}R^{d50}, —NR^{c50}R^{d50}, —NR^{c50}S(O)R^{b50}, —NR^{c50}S(O)NR^{c50}R^{d50}, —S(O)R^{b50}, —S(O)₂R^{b50}, —S(O)NR^{c50}R^{d50}, —S(O)₂NR^{c50}R^{d50}, —C(═NR^{e50})R^{a50}, —C(═NR^{e50})NR^{c50}R^{d50}, —NR^{c50}C(═NR^{e50})R^{a50}, —NR^{c50}C(═NR^{e50})NR^{c50}R^{d50}, —NR^{c50}S(O)(═NR^{e50})R^{b50}, —NR^{c50}S(O)(═NR^{e50})NR^{c50}R^{d50}, —OS(O)(═NR^{e50})R^{b50}, —S(O)(═NR^{e50})R^{b50}, —S(O)(═NR^{e50})NR^{c50}R^{d50}, —C(O)NR^{c50}S(O)₂R^{b50}, —C(O)NR^{c50}S(O)₂NR^{c50}R^{d50}, —S(O)₂NR^{c50}C(O)R^{b50}, —NR^{c50}S(O)NR^{c50}C(O)R^{b50}, and —P(O)R^{f50}R^{g50}, wherein the C₁₋₆ alkyl, C₂₋₆ alkenyl, C₂-6 alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁵⁰ are each optionally substituted with 1, 2, 3, 4, 5, or 6 independently selected R^{G} substituents;
each R^{a50}, R^{c50}, and R^{d50} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a50}, R^{c50} and R^{d50} are each optionally substituted with 1, 2, 3, 4, 5, or 6 independently selected R^{G} substituents;
or, any R^{c50} and R^{d50} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b50} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b5}° are each optionally substituted with 1, 2, 3, 4, 5, or 6 independently selected R^{G} substituents;
each R^{e50} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl; and
each R^{f50} and R^{g50} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl.

In some embodiments, R⁵⁰ and one R⁶, taken together with the atoms to which they are attached, form a Ring A which is selected from C₅₋₁₀ cycloalkyl, phenyl, 5-10 membered heterocycloalkyl, and 5-6 membered heteroaryl, wherein the C₅₋₁₀ cycloalkyl, phenyl, 5-10 membered heterocycloalkyl, and 5-6 membered heteroaryl group is optionally substituted with 1, 2, 3, 4, 5, or 6 independently selected R⁵ substituents;

In some embodiments, the compound of Formula I is a compound of Formula II:

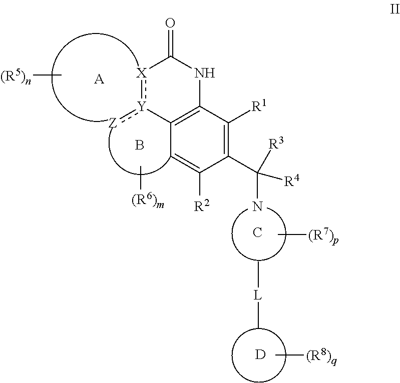

or a pharmaceutically acceptable salt thereof, wherein:
X is C or N;
Y is C or N;
Z is C or N;
[symbol] is a single or double bond;
n is 0, 1, 2, 3, 4, 5, or 6;
m is 0, 1, 2, 3, 4, 5, or 6;
p is 0, 1, 2, 3, 4, 5, or 6;
q is 0, 1, 2, 3, 4, 5, or 6;
Ring A is C₅₋₁₀ cycloalkyl, phenyl, 5-10 membered heterocycloalkyl, or 5-6 membered heteroaryl;
Ring B is C₅₋₁₀ cycloalkyl, phenyl, 5-10 membered heterocycloalkyl, or 5-6 membered heteroaryl;
Ring C is 4-14 membered heterocycloalkyl;
Ring D is C₃₋₁₀ cycloalkyl, C₆₋₁₀ aryl, 4-10 membered heterocycloalkyl, or 5-10 membered heteroaryl;
L is selected from bond, C₁₋₄ alkylene, C₁₋₄ haloalkylene, C₃₋₇ cycloalkylene, 4-7 membered heterocycloalkylene, —C₃₋₇ cycloalkylene-C₁₋₄ alkyl-, -(4-7 membered heterocycloalkylene)-C₁₋₄ alkyl-, —O—, —N(R^{L})—, —C(O)—, —C(O)N(R^{L})—, —N(R^{L})C(O)N(R^{L})—, —N(R^{L})C(O)O—, —S(O)₂—, —N(R^{L})S(O)₂—, and —N(R^{L})S(O)₂N(R^{L})—, wherein the C₁₋₄ alkylene, C₁₋₄ haloalkylene, C₃₋₇ cycloalkylene, 4-7 membered heterocycloalkylene, C₃₋₇ cycloalkylene-C₁₋₄ alkyl, and (4-7 membered heterocycloalkylene)-C₁₋₄ alkyl of L are each optionally substituted with 1, 2, 3, or 4 independently selected R^{G} substituents;
each R^{L} is independently selected from H, C₁₋₆ alkyl, and C₁₋₆ haloalkyl;
R¹ is selected from H, halo, CN, C₁₋₃ alkyl, C₁₋₃ haloalkyl, C₁₋₃ alkoxy, and C₁₋₃ haloalkoxy;
R² is selected from H, halo, CN, C₁₋₃ alkyl, C₁₋₃ haloalkyl, C₁₋₃ alkoxy, and C₁₋₃ haloalkoxy;
R³ is selected from H, halo, C₁₋₃ alkyl, and C₁₋₃ haloalkyl;
R⁴ is selected from H, halo, C₁₋₃ alkyl, and C₁₋₃ haloalkyl;
or, R³ and R⁴, together with the carbon atom to which they are attached, form a C₃₋₇ cycloalkyl, or 4-7 membered heterocycloalkyl group, wherein the C₃₋₇ cycloalkyl, and 4-7 membered heterocycloalkyl group is optionally substituted with 1, 2, 3, 4, 5, or 6 independently selected R^{G} substituents;
each R⁵ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{c5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R^{b5}, —NR^{c5}S(O)₂NR^{c5}R^{d5}, —NR^{c5}OR^{a5}, —NR^{c5}S(O)R^{b5}, —NR^{c5}S(O)NR^{c5}R^{d5}, —S(O)R^{b5}, —S(O)₂R^{b5}, —S(O)NR^{c5}R^{d5}, —S(O)₂NR^{c5}R^{d5}, —C(═NR^{e5})R^{a5}, —C(═NR^{e5})NR^{c5}R^{d5}, —NR^{c5}C(═NR^{e5})R^{a5}, —NR^{c5}C(═NR^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g5}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁵ are each optionally substituted with 1, 2, 3, 4, 5, or 6 independently selected R^{G} substituents;
each R^{a5}, R^{c5} and R^{d5} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a5}, R^{c5}, and R^{d5} are each optionally substituted with 1, 2, 3, 4, 5, or 6 independently selected R^{G} substituents;
or, any R^{c5} and R^{d5} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b5} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b5} are each optionally substituted with 1, 2, 3, 4, 5, or 6 independently selected R^{G} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁶ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6}, —SR^{a6}, —NR^{c6}R^{d6}, —NO₂, —C(O)R^{a6}, —C(O)OR^{a6}, —C(O)NR^{c6}R^{d6}, —C(O)NR^{c6}(OR^{a6}), —OC(O)R^{a6}, —OC(O)NR^{c6}R^{d6}, —OC(O)OR^{a6}, —OS(O)₂R^{b6}, —OS(O)₂NR^{c6}R^{d6}, —NR^{c6}C(O)R^{a6}, —NR^{c6}C(O)OR^{a6}, —NR^{c6}C(O)NR^{c6}R^{d6}, —NR^{c6}S(O)₂R^{b6}, —NR^{c6}S(O)₂NR^{c6}R^{d6}, —NR^{c6}OR^{a6}, —NR^{c6}S(O)R^{b6}, —NR^{c6}S(O)NR^{c6}R^{d6}, —S(O)R^{b6}, —S(O)₂R^{b6}, —S(O)NR^{c6}R^{d6}, —S(O)₂NR^{c6}R^{d6}, —C(═NR^{e6})R^{a6}, —C(═NR^{e6})R^{c6}R^{d6}, —R^{c6}C(═NR^{e6})R^{a6}, —NR^{c6}C(═NR^{e6})NR^{c6}R^{d6}, —NR^{c6}S(O)(═NR^{e6})R^{b6}, —NR^{c6}S(O)(═NR^{e6})NR^{c6}R^{d6}, —OS(O)(═NR^{e6})R^{b6}, —S(O)(═NR^{e6})R^{b6}, —S(O)(═NR^{e6})NR^{c6}R^{d6}, —C(O)NR^{c6}S(O)₂R^{b6}, —C(O)NR^{c6}S(O)₂NR^{c6}R^{d6}, —S(O)₂NR^{c6}C(O)R^{b6}, —NR^{c6}S(O)NR^{c6}C(O)R^{b6}, and —P(O)R^{f6}R^{g6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, 4, 5, or 6 independently selected R^{G} substituents;
each R^{a6}, R^{a6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a6}, R^{c6}, and R^{d6} are each optionally substituted with 1, 2, 3, 4, 5, or 6 independently selected R^{G} substituents;
or, any R^{c6} and R^{d6} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b6} are each optionally substituted with 1, 2, 3, 4, 5, or 6 independently selected R^{G} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f6} and R^{g6} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁷ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a7}, —SR^{a7}, —NR^{c7}R^{d7}, —NO₂, —C(O)R^{a7}, —C(O)OR^{a7}, —C(O)NR^{c7}R^{d7}, —C(O)NR^{c7}(OR^{a7}), —OC(O)R^{a7}, —OC(O)NR^{c7}R^{d7}, —OC(O)OR^{a7}, —OS(O)₂R^{b7}, —OS(O)₂NR^{c7}R^{d7}, —NR^{c7}C(O)R^{a7}, —NR^{c7}C(O)OR^{a7}, —NR^{c7}C(O)NR^{c7}R^{d7}, —NR^{c7}S(O)₂R^{b7}, —NR^{c7}S(O)₂NR^{c7}R^{d7}, —NR^{c7}OR^{a7}, —NR^{c7}S(O)R^{b7}, —NR^{c7}S(O)NR^{c7}R^{d7}, —S(O)R^{b7}, —S(O)₂R^{b7}, —S(O)NR^{c7}R^{d7}, —S(O)₂NR^{c7}R^{d7}, —C(═NR^{c7})R^{a7}, —C(═NR^{e7})NR^{c7}R^{d7}, —NR^{c7}C(═NR^{e7})R^{a7}, —NR^{c7}C(═NR^{e7})NR^{c7}R^{d7}, —NR^{c7}S(O)(═NR^{e7})R^{b7}, —NR^{c7}S(O)(═NR^{e7})NR^{c7}R^{d7}, —OS(O)(═NR^{e7})R^{b7}, —S(O)(═NR^{e7})R^{b7}, —S(O)(═NR^{e7})NR^{c7}R^{d7}, —C(O)NR^{c7}S(O)₂R^{b7}, —C(O)NR^{c7}S(O)₂NR^{c7}R^{d7}, —S(O)₂NR^{c7}C(O)R^{b7}, —NR^{c7}S(O)NR^{c7}C(O)R^{b7} and —P(O)R^{f7}R^{g7}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁷ are each optionally substituted with 1, 2, 3, 4, 5, or 6 independently selected R^{G} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a7}, R^{c7}, and R^{d7} are each optionally substituted with 1, 2, 3, 4, 5, or 6 independently selected R^{G} substituents;
or, any R^{c7} and R^{d7} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b7} are each optionally substituted with 1, 2, 3, 4, 5, or 6 independently selected R^{G} substituents;
each R^{c7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁸ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, —C(═NR^{e8})R^{a8}, —C(═NR^{e8})NR^{c8}R^{d8}, —NR^{c8}C(═NR^{e8})R^{a8}, —NR^{c8}C(═NR^{e8})NR^{c8}R^{d8}, —R^{c8}S(O)(═NR^{e8})R^{b8}, —NR^{c8}S(O)(═NR^{e8})NR^{c8}R^{d8}, —OS(O)(═NR^{e8})R^{b8}, —S(O)(═NR^{e8})R^{b8}, —S(O)(═NR^{e8})NR^{c8}R^{d8}, —C(O)NR^{c8}S(O)₂R^{b8}, —C(O)NR^{c8}S(O)₂NR^{c8}R^{d8}, —S(O)₂NR^{c8}C(O)R^{b8}, —NR^{c8}S(O)NR^{c8}C(O)R^{b8}, and —P(O)R^{f8}R^{g8}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁸ are each optionally substituted with 1, 2, 3, 4, 5, or 6 independently selected R^{8A} substituents;
or, a R⁷ and a R⁸, together with the atoms to which they are attached, form a C₅₋₁₀ cycloalkyl, phenyl, 5-10 membered heterocycloalkyl, or 5-6 membered heteroaryl group, wherein the C₅₋₁₀ cycloalkyl, phenyl, 5-10 membered heterocycloalkyl, or 5-6 membered heteroaryl group is optionally substituted with 1, 2, 3, 4, 5, or 6 independently selected R^{8A} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{c8}, and R^{d8} are each optionally substituted with 1, 2, 3, 4, 5, or 6 independently selected R^{8A} substituents;
or, any R^{c8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{b8} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8} are each optionally substituted with 1, 2, 3, 4, 5, or 6 independently selected R^{8A} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{c8A}R^{d8A}, —NO₂, —C(O)R^{a8A}, —C(O)OR^{a8A}, —C(O)NR^{c8A}R^{d8A}, —C(O)NR^{c8A}(OR^{a8A}), —OC(O)R^{a8A}, —OC(O)NR^{c8A}R^{d8A}, —OC(O)OR^{a8A}, —OS(O)₂R^{b8A}, —OS(O)₂NR^{c8A}R^{d8A}, —NR^{c8A}C(O)R^{a8A}, —NR^{c8A}C(O)OR^{a8A}, —NR^{c8A}C(O)NR^{c8A}R^{d8A}, —NR^{c8A}S(O)₂R^{b8A}, —NR^{c8A}S(O)₂NR^{c8A}R^{d8A}, —NR^{e8A}OR^{a8A}, —NR^{c8A}S(O)R^{b8A}, —NR^{c8A}S(O)NR^{c8A}R^{d8A}, —S(O)R^{b8A}, —S(O)₂R^{b8A}, —S(O)NR^{c8A}R^{d8A}, —S(O)₂NR^{c8A}R^{d8A}, C(═NR^{e8A})R^{b8A}, —C(═NR^{c8A})R^{c8A}R^{d8A}, —NR^{c8A}C(═NR^{c8A})R^{a8A}, —NR^{c8A}C(═NR^{c8A})NR^{c8A}R^{d8A}, —NR^{e8A}S(O)(═NR^{c8A})R^{b8A}, —NR^{e8A}S(O)(═NR^{c8A})NR^{c8A}R^{d8A}, —OS(O)(═NR^{c8A})R^{b8A}, —S(O)(═NR^{c8A})R^{b8A}, —S(O)(═NR^{c8A})R^{c8A}R^{d8A}, —C(O)NR^{e8A}S(O)₂R^{b8A}, —C(O)NR^{c8A}S(O)₂NR^{c8A}R^{d8A}, —S(O)₂NR^{c8A}C(O)R^{b8A}, —NR^{e8A}S(O)NR^{c8A}C(O)R^{b8A}, and —P(O)R^{f8A}R^{g8A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{8A} are each optionally substituted with 1, 2, 3, 4, 5, or 6 independently selected R^{G} substituents;
each R^{a8A}, R^{c8A}, and R^{d8A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8A}, R^{c8A} and R^{d8A} are each optionally substituted with 1, 2, 3, 4, 5, or 6 independently selected R^{G} substituents;
or, any R^{c8A} and R^{d8A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b8A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8A} are each optionally substituted with 1, 2, 3, 4, 5, or 6 independently selected R^{G} substituents;
each R^{e8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁. 3 alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, X is N.

In some embodiments, X is C.

In some embodiments, Y is C.

In some embodiments, Y is N.

In some embodiments, X is N and Y is C.

In some embodiments, Z is N.

In some embodiments, Z is C.

In some embodiments, X is N, Y is C, and Z is N.

In some embodiments, X is N, Y is C, and Z is C.

In some embodiments, Ring A is C₅₋₇ cycloalkyl, phenyl, 5-7 membered heterocycloalkyl, or 5-6 membered heteroaryl.

In some embodiments, Ring A is 5-10 membered heterocycloalkyl.

In some embodiments, Ring A is 5-7 membered heterocycloalkyl.

In some embodiments, Ring A is 6-membered heterocycloalkyl.

In some embodiments, Ring A is selected from

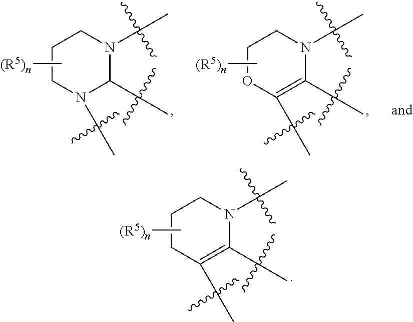

In some embodiments, Ring A is

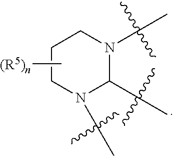

In some embodiments, Ring A is

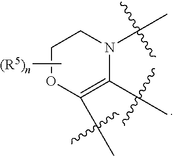

In some embodiments, Ring A is

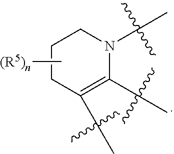

In some embodiments, n is 0, 1, 2, 3, or 4.

In some embodiments, n is 0, 1, or 2.

In some embodiments, n is 0 or 1.

In some embodiments, n is 0.

In some embodiments, Ring B is C₅₋₇ cycloalkyl, phenyl, 5-7 membered heterocycloalkyl, or 5-6 membered heteroaryl.

In some embodiments, Ring B is 5-10 membered heterocycloalkyl or 5-6 membered heteroaryl.

In some embodiments, Ring B is 5-7 membered heterocycloalkyl or 5-6 membered heteroaryl.

In some embodiments, Ring B is 5-6 membered heteroaryl.

In some embodiments, Ring B is 5-membered heteroaryl.

In some embodiments, Ring B is 6-membered heteroaryl.

In some embodiments, Ring B is selected from A and

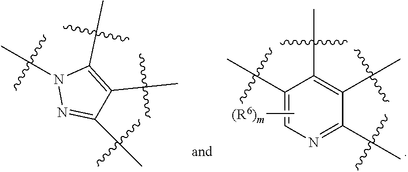

In some embodiments, Ring B is

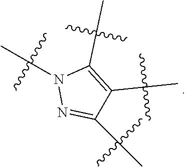 [SMILES: *c1nn(*)c(*)c1*]

In some embodiments, Ring B is

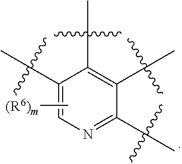

In some embodiments, m is 0, 1, 2, 3, or 4.

In some embodiments, m is 0, 1, or 2.

In some embodiments, m is 0 or 1.

In some embodiments, m is 0.

In some embodiments, R¹ is selected from H, halo, C₁₋₃ alkyl, and C₁₋₃ haloalkyl; In some embodiments, R¹ is selected from H, halo, and C₁₋₃ alkyl.

In some embodiments, R¹ is halo.

In some embodiments, R¹ is fluoro. In some embodiments, R² is selected from H, halo, and C₁₋₃ alkyl.

In some embodiments, R² is selected from H and C₁₋₃ alkyl.

In some embodiments, R² is selected from H.

In some embodiments, R³ is selected from H, halo, and C₁₋₃ alkyl.

In some embodiments, R³ is selected from H and C₁₋₃ alkyl.

In some embodiments, R³ is H.

In some embodiments, R⁴ is selected from H, halo, and C₁₋₃ alkyl.

In some embodiments, R⁴ is selected from H and C₁₋₃ alkyl.

In some embodiments, R⁴ is H.

In some embodiments, R³ and R⁴ are each H.

In some embodiments, Ring C is 4-10 membered heterocycloalkyl.

In some embodiments, Ring C is 4-7 membered heterocycloalkyl.

In some embodiments, Ring C is 6-membered heterocycloalkyl.

In some embodiments, Ring C is piperazinyl.

In some embodiments, Ring C is

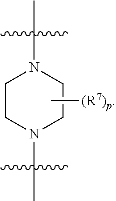

In some embodiments, p is 0, 1, 2, 3, or 4.

In some embodiments, p is 0, 1, or 2.

In some embodiments, p is 0 or 1.

In some embodiments p is 0.

In some embodiments, Ring D is 5-10 membered heteroaryl.

In some embodiments, Ring D is pyridinyl.

In some embodiments, L is selected from bond, —O—, and —N(R^{L})—.

In some embodiments, L is a bond.

In some embodiments, L is —O—.

In some embodiments, L is —N(R^{L})—.

In some embodiments, q is 0, 1, 2, 3, 4, or 5.

In some embodiments, q is 1, 2, 3, 4, or 5.

In some embodiments, q is 1, 2, 3, or 4.

In some embodiments, q is 1, 2, or 3.

In some embodiments, q is 1 or 2.

In some embodiments, q is 2. In some embodiments, each R⁸ is independently selected from halo, C₁₋₆ alkyl, C₂₋₆ alkenyl, C₂₋₆ alkynyl, C₁₋₆ haloalkyl, C₃₋₁₀ cycloalkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, —C(═NR^{e8})R^{a8}, —C(═NR^{e8})NR^{c8}R^{d8}, —NR^{c8}C(═NR^{e8})R^{a8}, —NR^{c8}C(═NR^{e8})NR^{c8}R^{d8}, —NR^{c8}S(O)(═NR^{e8})R^{b8}, —NR^{c8}S(O)(═NR^{e8})NR^{c8}R^{d8}, —OS(O)(═NR^{e8})R^{b8}, —S(O)(═NR^{e8})R^{b8}, —S(O)(═NR^{e8})NR^{c8}R^{d8}, —C(O)NR^{c8}S(O)₂R^{b8}, —C(O)NR^{c8}S(O)₂NR^{c8}R^{d8}, —S(O)₂NR^{c8}C(O)R^{b8}, —NR^{c8}S(O)NR^{c8}C(O)R^{b8}, and —P(O)R^{f8}R^{g8}, wherein the C₁₋₆ alkyl, C₂₋₆ alkenyl, C₂₋₆ alkynyl, and C₁₋₆ haloalkyl, of R⁸ are each optionally substituted with 1, 2, 3, 4, 5, or 6 independently selected R^{8A} substituents.

In some embodiments, each R⁸ is independently selected from halo, C₁₋₆ alkyl, C₂₋₆ alkenyl, C₂₋₆ alkynyl, C₁₋₆ haloalkyl, C₃₋₁₀ cycloalkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, and —P(O)R^{f8}R^{g8}, wherein the C₁₋₆ alkyl, C₂₋₆ alkenyl, C₂₋₆ alkynyl, and C₁₋₆ haloalkyl, of R⁸ are each optionally substituted with 1, 2, 3, 4, 5, or 6 independently selected R^{8A} substituents.

In some embodiments, each R⁸ is independently selected from halo, C₁₋₆ alkyl, C₂₋₆ alkenyl, C₂₋₆ alkynyl, C₁₋₆ haloalkyl, —CN, —OR^{a8}, —NR^{c8}R^{d8}, —C(O)R^{a8}, —C(O)OR^{a8}, —C(O)NR^{c8}R^{d8}, and —NR^{c8}C(O)R^{a8}.

In some embodiments, each R⁸ is independently selected from C₁₋₆ alkyl, C₁₋₆ haloalkyl, —C(O)NR^{c8}R^{d8}, and —NR^{c8}C(O)R^{a8}.

In some embodiments, each R⁸ is independently selected from C₁₋₆ alkyl and —C(O)NR^{c8}R^{d8}.

In some embodiments, each R⁸ is independently selected from C₁₋₃ alkyl and —C(O)NR^{c8}R^{d8}.

In some embodiments, each R^{a8}, R^{b8}, R^{c8}, R^{d8}, R^{e8}, R^{f8}, and R^{g8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl.

In some embodiments, each R^{a8}, R^{b8}, R^{c8}, R^{d8}, R^{e8}, R^{f8}, and R^{g8} is independently selected from H, C₁₋₆ alkyl, and C₃₋₇ cycloalkyl.

In some embodiments, each R^{b8} and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl.

In some embodiments, each R^{c8} and R^{d8} is independently selected from H, C₁₋₆ alkyl, and C₃₋₇ cycloalkyl.

In some embodiments, each R⁸ is independently selected from C₁₋₆ alkyl and —C(O)NR^{c8}R^{d8}; and
each R^{c8} and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl.

In some embodiments, each R⁸ is independently selected from C₁₋₃ alkyl and —C(O)NR^{c8}R^{d8}; and
each R^{c8} and R^{d8} is independently selected from H, C₁₋₆ alkyl, and C₃₋₇ cycloalkyl.

In some embodiments, each R⁸ is independently selected from methyl, methylaminocarbonyl, and cyclopropylaminocarbonyl.

In some embodiments:

X is C or N;
[symbol] is a single or double bond;
m is 0, 1, 2, 3, or 4;
p is 0, 1, 2, 3, or 4;
q is 0, 1, 2, 3, or 4;
Ring B is 5-6 membered heteroaryl;
Ring C is 4-10 membered heterocycloalkyl;
Ring D is 5-10 membered heteroaryl;
L is selected from bond, —O—, and —N(R^{L})—;
each R^{L} is independently selected from H, C₁₋₆ alkyl, and C₁₋₆ haloalkyl;
R¹ is selected from H, halo, and C₁₋₃ alkyl;
R² is selected from H, halo, and C₁₋₃ alkyl;
R³ is selected from H, halo, and C₁₋₃ alkyl;
R⁴ is selected from H, halo, and C₁₋₃ alkyl;
R⁵⁰ and one R⁶, taken together with the atoms to which they are attached, form a Ring A which is selected from C₅₋₁₀ cycloalkyl, phenyl, 5-10 membered heterocycloalkyl;
each R⁸ is independently selected from oxo, halo, C₁₋₆ alkyl, C₂₋₆ alkenyl, C₂₋₆ alkynyl, C₁₋₆ haloalkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, —C(═NR^{e8})R^{a8}, —C(═NR^{e8})NR^{c8}R^{d8}, —NR^{c8}C(═NR^{e8})R^{a8}, —NR^{c8}C(═NR^{e8})NR^{c8}R^{d8}, —NR^{c8}S(O)(═NR^{e8})R^{b8}, —NR^{c8}S(O)(═NR^{e8})NR^{c8}R^{d8}, —OS(O)(═NR^{e8})R^{b8}, —S(O)(═NR^{e8})R^{b8}, —S(O)(═NR^{e8})NR^{c8}R^{d8}, —C(O)NR^{c8}S(O)₂R^{b8}, —C(O)NR^{c8}S(O)₂NR^{c8}R^{d8}, —S(O)₂NR^{c8}C(O)R^{b8}, —NR^{c8}S(O)NR^{c8}C(O)R^{b8}, and —P(O)R^{f8}R^{g8}, wherein the C₁₋₆ alkyl, C₂₋₆ alkenyl, C₂₋₆ alkynyl, and C₁₋₆ haloalkyl of R⁸ are each optionally substituted with 1, 2, 3, 4, 5, or 6 independently selected R^{c8A} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{c8}, and R^{d8} are each optionally substituted with 1, 2, 3, 4, 5, or 6 independently selected R^{8A} substituents;
or, any R^{c8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{b8} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₆ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8} are each optionally substituted with 1, 2, 3, 4, 5, or 6 independently selected R^{8A} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{c8A}R^{d8A}, —NO₂, —C(O)R^{a8A}, —C(O)OR^{a8A}, —C(O)NR^{c8A}R^{d8A}, —C(O)NR^{c8A}(OR^{a8A}), —OC(O)R^{a8A}, —OC(O)NR^{c8A}R^{d8A}, —OC(O)OR^{a8A}, —OS(O)₂R^{b8A}, —OS(O)₂NR^{c8A}R^{d8A}, —NR^{c8A}C(O)R^{a8A}, —NR^{c8A}C(O)OR^{a8A}, —NR^{c8A}C(O)NR^{c8A}R^{d8A}, —NR^{c8A}S(O)₂R^{b8A}, —NR^{c8A}S(O)₂NR^{c8A}R^{d8A}, —NR^{e8A}OR^{a8A}, —NR^{c8A}S(O)R^{b8A}, —NR^{e8A}S(O)NR^{c8A}R^{d8A}, —S(O)R^{b8A}, —S(O)₂R^{b8A}, —S(O)NR^{c8A}R^{d8A}, —S(O)₂NR^{c8A}R^{d8A}, C(═NR^{c8A})R^{b8A}, —C(═NR^{c8A})R^{c8A}R^{d8A}, —NR^{c8A}C(═NR^{c8A})R^{a8A}, —NR^{c8A}C(═NR^{c8A})NR^{c8A}R^{d8A}, —NR^{e8A}S(O)(═NR^{c8A})R^{b8A}, —NR^{e8A}S(O)(═NR^{c8A})NR^{c8A}R^{d8A}, —OS(O)(═NR^{c8A})R^{b8A}, —S(O)(═NR^{c8A})R^{b8A}, —S(O)(═NR^{c8A})R^{c8A}R^{d8A}, —C(O)NR^{e8A}S(O)₂R^{b8A}, —C(O)NR^{c8A}S(O)₂NR^{c8A}R^{d8A}, —S(O)₂NR^{c8A}C(O)R^{b8A}, —NR^{e8A}S(O)NR^{c8A}C(O)R^{b8A} and —P(O)R^{f8A}R^{g8A};
each R^{a8A}, R^{b8A}, and R^{d8A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{b8A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and
each R^{f8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl.

In some embodiments:
X is C or N;
[symbol] is a single or double bond;
m is 0, 1, 2, 3, or 4;
p is 0, 1, 2, 3, or 4;
q is 0, 1, 2, 3, or 4;
Ring B is 5-6 membered heteroaryl;
Ring C is 4-10 membered heterocycloalkyl;
Ring D is 5-10 membered heteroaryl;
L is selected from bond, —O—, and —N(R^{L})—;
each R^{L} is independently selected from H, C₁₋₆ alkyl, and C₁₋₆ haloalkyl;
R¹ is selected from H, halo, and C₁₋₃ alkyl;
R² is selected from H, halo, and C₁₋₃ alkyl;
R³ is selected from H, halo, and C₁₋₃ alkyl;
R⁴ is selected from H, halo, and C₁₋₃ alkyl;
R⁵⁰ and one R⁶, taken together with the atoms to which they are attached, form a Ring A which is selected from 5-7 membered heterocycloalkyl;
each R⁸ is independently selected from halo, C₁₋₆ alkyl, C₂₋₆ alkenyl, C₂₋₆ alkynyl, C₁₋₆ haloalkyl, —CN, —OR^{a8}, —NR^{c8}R^{d8}, —C(O)R^{a8}, —C(O)OR^{a8}, —C(O)NR^{c8}R^{d8}, and —NR^{c8}C(O)R^{a8};
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl.

In some embodiments, the compound of Formula I is a compound of Formula II, wherein:
X is C or N;
Y is C or N;
Z is C or N;
[symbol] s a single or double bond;
n is 0, 1, 2, 3, or 4;
p is 0, 1, 2, 3, or 4;
p is 0, 1, 2, 3, or 4;
q is 0, 1, 2, 3, or 4;
Ring A is 5-10 membered heterocycloalkyl;
Ring B is 5-6 membered heteroaryl;
Ring C is 4-10 membered heterocycloalkyl;
Ring D is 5-10 membered heteroaryl;
L is selected from bond, —O—, and —N(R^{L})—;
each R^{L} is independently selected from H, C₁₋₆ alkyl, and C₁₋₆ haloalkyl;
R¹ is selected from H, halo, and C₁₋₃ alkyl;
R² is selected from H, halo, and C₁₋₃ alkyl;
R³ is selected from H, halo, and C₁₋₃ alkyl;
R⁴ is selected from H, halo, and C₁₋₃ alkyl;
each R⁸ is independently selected from oxo, halo, C₁₋₆ alkyl, C₂₋₆ alkenyl, C₂₋₆ alkynyl, C₁₋₆ haloalkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, —C(═NR^{e8})R^{a8}, —C(═NR^{e8})NR^{c8}R^{d8}, —NR^{c8}C(═NR^{e8})R^{a8}, —NR^{c8}C(═NR^{e8})NR^{c8}R^{d8}, —NR^{c8}S(O)(═NR^{e8})R^{b8}, —NR^{c8}S(O)(═NR^{e8})NR^{c8}R^{d8}, —OS(O)(═NR^{e8})R^{b8}, —S(O)(═NR^{e8})R^{b8}, —S(O)(═NR^{e8})NR^{c8}R^{d8}, —C(O)NR^{c8}S(O)₂R^{b8}, —C(O)NR^{c8}S(O)₂NR^{c8}R^{d8}, —S(O)₂NR^{c8}C(O)R^{b8}, —NR^{c8}S(O)NR^{c8}C(O)R^{b8}, and —P(O)R^{f8}R^{g8}, wherein the C₁₋₆ alkyl, C₂₋₆ alkenyl, C₂₋₆ alkynyl, and C₁₋₆ haloalkyl of R⁸ are each optionally substituted with 1, 2, 3, 4, 5, or 6 independently selected R^{c8A} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{c8}, and R^{d8} are each optionally substituted with 1, 2, 3, 4, 5, or 6 independently selected R^{8A} substituents;
or, any R^{c8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{b8} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8} are each optionally substituted with 1, 2, 3, 4, 5, or 6 independently selected R^{8A} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{c8A}R^{d8A}, —NO₂, —C(O)R^{a8A}, —C(O)OR^{a8A}, —C(O)NR^{c8A}R^{d8A}, —C(O)NR^{c8A}(OR^{a8A}), —OC(O)R^{a8A}, —OC(O)NR^{c8A}R^{d8A}, —OC(O)OR^{a8}, —OS(O)₂R^{b8A}, —OS(O)₂NR^{c8A}R^{d8A}, —NR^{c8A}C(O)R^{a8A}, —NR^{c8A}C(O)OR^{a8A}, —NR^{c8A}C(O)NR^{c8A}R^{d8A}, —NR^{c8A}S(O)₂R^{b8A}, —R^{e8A}S(O)₂NR^{c8A}R^{d8A}, —NR^{e8A}OR^{a8A}, —NR^{e8A}S(O)R^{b8A}, —NR^{c8A}S(O)NR^{c8A}R^{d8A}, —S(O)R^{b8A}, —S(O)₂R^{b8A}, —S(O)NR^{c8A}R^{d8A}, —S(O)₂NR^{c8A}R^{d8A}, C(═R^{c8A})R^{b8A}, —C(═NR^{c8A})R^{c8A}R^{d8A}, —NR^{c8A}C(═NR^{c8A})R^{a8A}, —NR^{c8A}C(═NR^{e8A})NR^{c8A}R^{d8A}, —R^{c8A}S(O)(═NR^{c8A})R^{b8A}, —NR^{e8A}S(O)(═NR^{c8A})NR^{c8A}R^{d8A}, —OS(O)(═NR^{c8A})R^{b8A}, —S(O)(═NR^{c8A})R^{b8A}, —S(O)(═NR^{c8A})R^{c8A}R^{d8A}, —C(O)NR^{c8A}S(O)₂R^{b8A}, —C(O)NR^{c8A}S(O)₂NR^{c8A}R^{d8A}, —S(O)₂NR^{c8A}C(O)R^{b8A}, —NR^{e8A}S(O)NR^{c8A}C(O)R^{b8A} and —P(O)R^{f8A}R^{g8A};
each R^{a8A}, R^{c8A}, and R^{d8A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{b8A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{e8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and
each R^{f8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl.

In some embodiments, the compound of Formula I is a compound of Formula II, wherein:
X is C or N;
Y is C or N;
Z is C or N;
[symbol] is a single or double bond;
n is 0, 1, 2, 3, or 4;
m is 0, 1, 2, 3, or 4;
p is 0, 1, 2, 3, or 4;
q is 0, 1, 2, 3, or 4;
Ring A is 5-10 membered heterocycloalkyl;
Ring B is 5-6 membered heteroaryl;
Ring C is 4-10 membered heterocycloalkyl;
Ring D is 5-10 membered heteroaryl;
L is selected from bond, —O—, and —N(R^{L})—;
each R^{L} is independently selected from H, C₁₋₆ alkyl, and C₁₋₆ haloalkyl;
R¹ is selected from H, halo, and C₁₋₃ alkyl;
R² is selected from H, halo, and C₁₋₃ alkyl;
R³ is selected from H, halo, and C₁₋₃ alkyl;
R⁴ is selected from H, halo, and C₁₋₃ alkyl;
each R⁸ is independently selected from halo, C₁₋₆ alkyl, C₂₋₆ alkenyl, C₂₋₆ alkynyl, C₁₋₆ haloalkyl, —CN, —OR^{a8}, —R^{c8}R^{d8}, —C(O)R^{a8}, —C(O)OR^{a8}, —C(O)NR^{c8}R^{d8}, and —NR^{c8}C(O)R^{a8};
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl.

In some embodiments, the compound of Formula I is a compound of Formula IIa:

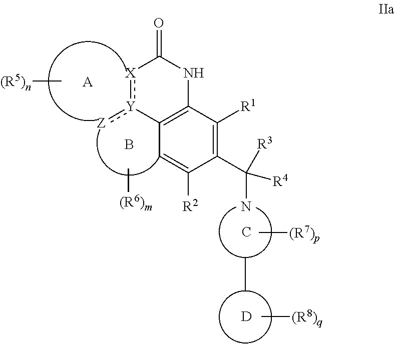

or a pharmaceutically acceptable salt thereof.

In some embodiments, the compound of Formula I is a compound of Formula III:

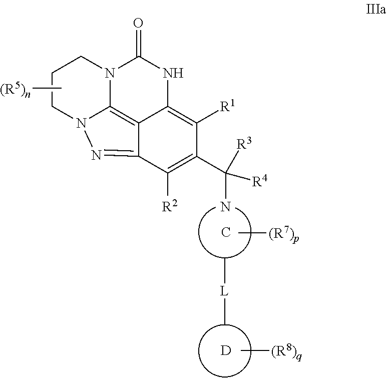

or a pharmaceutically acceptable salt thereof.

In some embodiments, the compound of Formula I is a compound of Formula IIIa:

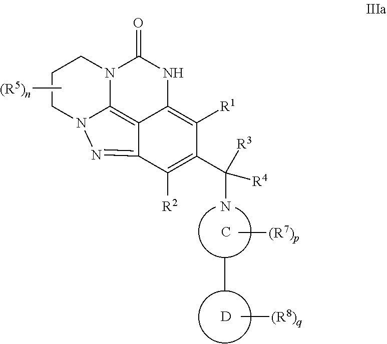

or a pharmaceutically acceptable salt thereof.

In some embodiments, the compound of Formula I is a compound of Formula IIIb:

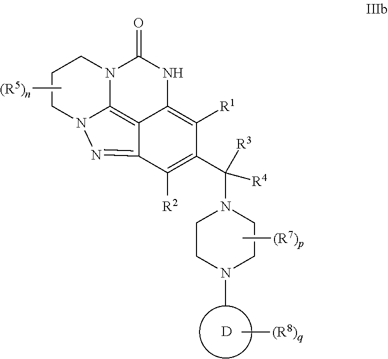

or a pharmaceutically acceptable salt thereof.

In some embodiments, the compound of Formula I is a compound of Formula IV:

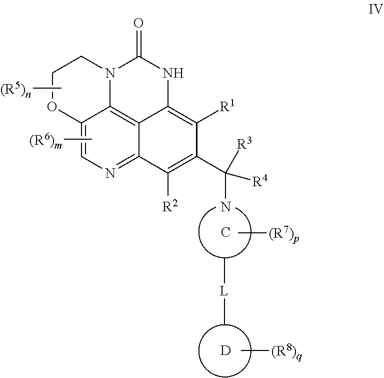

or a pharmaceutically acceptable salt thereof.

In some embodiments, the compound of Formula I is a compound of Formula IVa:

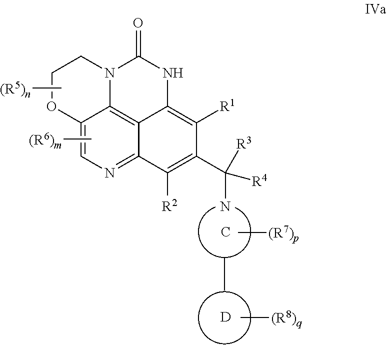

or a pharmaceutically acceptable salt thereof.

In some embodiments, the compound of Formula I is a compound of Formula IVb:

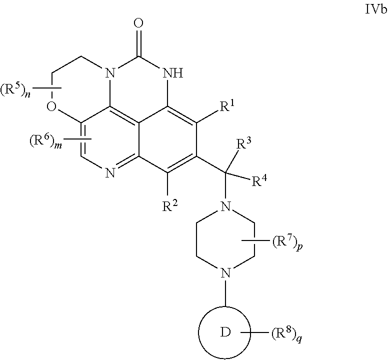

or a pharmaceutically acceptable salt thereof.

In some embodiments, the compound of Formula I is a compound of Formula V:

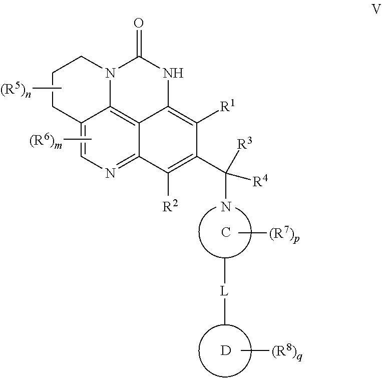

or a pharmaceutically acceptable salt thereof.

In some embodiments, the compound of Formula I is a compound of Formula Va:

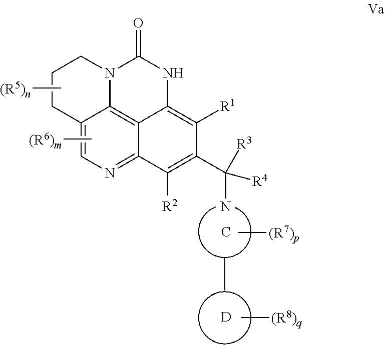

or a pharmaceutically acceptable salt thereof.

In some embodiments, the compound of Formula I is a compound of Formula Vb:

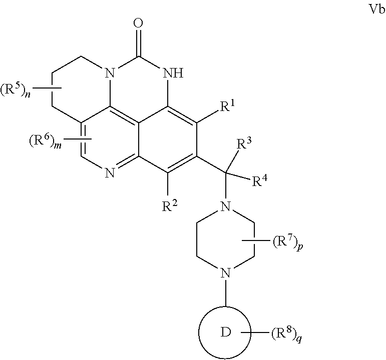

or a pharmaceutically acceptable salt thereof.

In some embodiments, the compound provided herein is selected from:
5-(4-((7-fluoro-9-oxo-2,3,8,9-tetrahydro-1H-3a,4,8,9a-tetraazacyclopenta[def]phenanthren-6-yl)methyl)piperazin-1-yl)-N,6-dimethylpicolinamide;
N-cyclopropyl-5-(4-((7-fluoro-9-oxo-2,3,8,9-tetrahydro-1H-3a,4,8,9a-tetraazacyclopenta[def]phenanthren-6-yl)methyl)piperazin-1-yl)-6-methylpicolinamide;
5-(4-((6-fluoro-4-oxo-2,3,4,5-tetrahydro-1-oxa-3a,5,9-triazapyren-7-yl)methyl)piperazin-1-yl)-N,6-dimethylpicolinamide;
N-cyclopropyl-5-(4-((6-fluoro-4-oxo-2,3,4,5-tetrahydro-1-oxa-3a,5,9-triazapyren-7-yl)methyl)piperazin-1-yl)-6-methylpicolinamide;
5-(4-((7-fluoro-5-oxo-2,3,5,6-tetrahydro-1H-[1,6]naphthyridino[1,8,7-cde]quinazolin-8-yl)methyl)piperazin-1-yl)-N,6-dimethylpicolinamide; and
N-cyclopropyl-5-(4-((7-fluoro-5-oxo-2,3,5,6-tetrahydro-1H-[1,6]naphthyridino[1,8,7-cde]quinazolin-8-yl)methyl)piperazin-1-yl)-6-methylpicolinamide;
or a pharmaceutically acceptable salt thereof.

In some embodiments, the compound provided herein is selected from:
5-(4-((3-ethyl-9-fluoro-2-oxo-2,3-dihydro-1H-pyrimido[4,5,6-de]quinazolin-8-yl)methyl)piperazin-1-yl)-N-(2-hydroxyethyl)-6-methylpicolinamide;
N-(2-cyanoethyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-(cyanomethyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-N-((1r,3r)-3-hydroxycyclobutyl)-6-methylpicolinamide;
5-(4-((3-ethyl-2-oxo-2,3-dihydro-1H-pyrimido[4,5,6-de]quinazolin-8-yl)methyl)piperazin-1-yl)-N-(2-hydroxyethyl)-6-methylpicolinamide;
N-(2-cyanoethyl)-5-(4-((3-ethyl-2-oxo-2,3-dihydro-1H-pyrimido[4,5,6-de]quinazolin-8-yl)methyl)piperazin-1-yl)-6-methylpicolinamide;
N-(cyanomethyl)-5-(4-((3-ethyl-2-oxo-2,3-dihydro-1H-pyrimido[4,5,6-de]quinazolin-8-yl)methyl)piperazin-1-yl)-6-methylpicolinamide;
N-cyclopropyl-5-(4-((3-ethyl-2-oxo-2,3-dihydro-1H-pyrimido[4,5,6-de]quinazolin-8-yl)methyl)piperazin-1-yl)-6-methylpicolinamide;
N-(1-cyanocyclopropyl)-5-(4-((3-ethyl-2-oxo-2,3-dihydro-1H-pyrimido[4,5,6-de]quinazolin-8-yl)methyl)piperazin-1-yl)-6-methylpicolinamide;
N-cyclobutyl-5-(4-((3-ethyl-2-oxo-2,3-dihydro-1H-pyrimido[4,5,6-de]quinazolin-8-yl)methyl)piperazin-1-yl)-6-methylpicolinamide;
5-(4-((3-ethyl-2-oxo-2,3-dihydro-1H-pyrimido[4,5,6-de]quinazolin-8-yl)methyl)piperazin-1-yl)-N-((1s,3s)-3-hydroxycyclobutyl)-6-methylpicolinamide;
5-(4-((3-ethyl-2-oxo-2,3-dihydro-1H-pyrimido[4,5,6-de]quinazolin-8-yl)methyl)piperazin-1-yl)-N-((1r,3r)-3-hydroxycyclobutyl)-6-methylpicolinamide;
N-((1R,5S,6r)-3-oxabicyclo[3.1.0]hexan-6-yl)-5-(4-((3-ethyl-2-oxo-2,3-dihydro-1H-pyrimido[4,5,6-de]quinazolin-8-yl)methyl)piperazin-1-yl)-6-methylpicolinamide;
N-((1r,3r)-3-cyanocyclobutyl)-5-(4-((3-ethyl-2-oxo-2,3-dihydro-1H-pyrimido[4,5,6-de]quinazolin-8-yl)methyl)piperazin-1-yl)-6-methylpicolinamide;
N-((1s,3s)-3-cyanocyclobutyl)-5-(4-((3-ethyl-2-oxo-2,3-dihydro-1H-pyrimido[4,5,6-de]quinazolin-8-yl)methyl)piperazin-1-yl)-6-methylpicolinamide;
N-(1-(cyanomethyl)cyclopropyl)-5-(4-((3-ethyl-2-oxo-2,3-dihydro-1H-pyrimido[4,5,6-de]quinazolin-8-yl)methyl)piperazin-1-yl)-6-methylpicolinamide;
N-cyclopropyl-5-(4-((3-cyclopropyl-2-oxo-2,3-dihydro-1H-pyrimido[4,5,6-de]quinazolin-8-yl)methyl)piperazin-1-yl)-6-methylpicolinamide; and
5-(4-((3-(cyanomethyl)-2-oxo-2,3-dihydro-1H-pyrimido[4,5,6-de]quinazolin-8-yl)methyl)piperazin-1-yl)-N-cyclopropyl-6-methylpicolinamide;
   or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —N(R^{L})C(O)— includes both —N(R^{L})C(O)— and —C(O)N(R^{L})— (e.g. —NHC(O)— includes both —NHC(O)— and —C(O)NH—). Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the terms “Cₙ₋ₘ” and “Cₘ₋ₙ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, from 2 to 6 carbon atoms, from 2 to 4 carbon atoms, from 2 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, and the like. In some embodiments, aryl groups have from 5 to 10 carbon atoms. In some embodiments, the aryl group is phenyl or naphthyl. In some embodiments, the aryl is phenyl.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula —O-haloalkyl having n to m carbon atoms. Example haloalkoxy groups include OCF₃ and OCHF₂.

In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-, 7-, 8-, 9-, or 10-membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-, 7-, 8-, 9-, or 10-membered monocyclic or bicyclic heteroaryl having 1, 2, 3, or 4 heteroatom ring members independently selected from N, O, and S. In some embodiments, the heteroaryl is a 5-6 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, and S. In some embodiments, the heteroaryl group contains 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, indazolyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 10 ring-forming atoms, 4 to 8 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, tetrahydrothiopheneyl, tetrahydrothiopheneyl 1,1-dioxide, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, 2-azaspiro[3.3]heptanyl, diazaspiro[3.3]heptanyl, azaspiro[3.5]nonanyl, 7-azaspiro[3.5]nonanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 3-oxopiperazinyl, oxo-pyrrolidinyl, oxo-pyridinyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” or “alkylene linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, a “haloalkyl linking group” or “haloalkylene linking group” is a bivalent straight chain or branched haloalkyl linking group (“haloalkylene group”). Example haloalkylene groups include —CF₂—, —C₂F₄—, —CHF—, —CCl₂—, —CHCl—, —C₂Cl₄—, and the like.

As used herein, a “cycloalkyl linking group” or “cycloalkylene linking group” is a bivalent straight chain or branched cycloalkyl linking group (“cycloalkylene group”). Examples of “cycloalkyl linking groups” or “cycloalkylene groups” include cyclopropy-1,1,-diyl, cyclopropy-1,2-diyl, cyclobut-1,3,-diyl, cyclopent-1,3,-diyl, cyclopent-1,4,-diyl, cyclohex-1,2,-diyl, cyclohex-1,3,-diyl, cyclohex-1,4,-diyl, and the like.

As used herein, a “heterocycloalkyl linking group” or “heterocycloalkylene linking group” is a bivalent straight chain or branched heterocycloalkyl linking group (“heterocycloalkylene group”). Examples of “heterocycloalkyl linking groups” or “heterocycloalkylene groups” include azetidin-1,2-diyl, azetidin-1,3-diyl, pyrrolidin-1,2-diyl, pyrrolidin-1,3-diyl, pyrrolidin-2,3-diyl, piperidin-1,2-diyl, piperidin-1,3-diyl, piperidin-1,4-diyl, piperidin-2,3-diyl, piperidin-2,4-diyl, and the like.

As used herein, a “heteroaryl linking group” or “heteroarylene linking group” is a bivalent straight chain or branched heteroaryl linking group (“heteroarylene group”). Examples of “heteroaryl linking groups” or “heteroarylene groups” include pyrazol-1,3-diyl, imidazol-1,2,-diyl, pyridin-2,3-diyl, pyridin-2,4-diyl, pyridin-3,4-diyl, and the like.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{G}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula II, etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

Compounds of the invention, including salts thereof, can be prepared using known organic synthesis techniques and according to various possible synthetic routes. Example synthetic methods for preparing compounds of the invention are provided in the Schemes below.

Intermediates (e.g., compounds of Formula Int) are prepared, for example, according to the procedures shown in Scheme I: When L is bond and W is N, intermediate Int-3 is prepared by a process comprising reacting compounds of Formulae Int-1 and Int-2 under suitable conditions (e.g., nucleophilic aromatic substitution, Buchwald-Hartwig cross-coupling, Ullmann coupling, or Chan-Lam coupling). When L is bond and W is C, intermediate Int-6 is prepared by a process comprising reacting compounds of Formulae Int-4 and Int-5 under suitable conditions (e.g., Suzuki, Stille, or Negishi coupling). Compounds of Formula Int are prepared by treating intermediate Int-3 or Int-6 under suitable conditions (e.g., removal of PG).

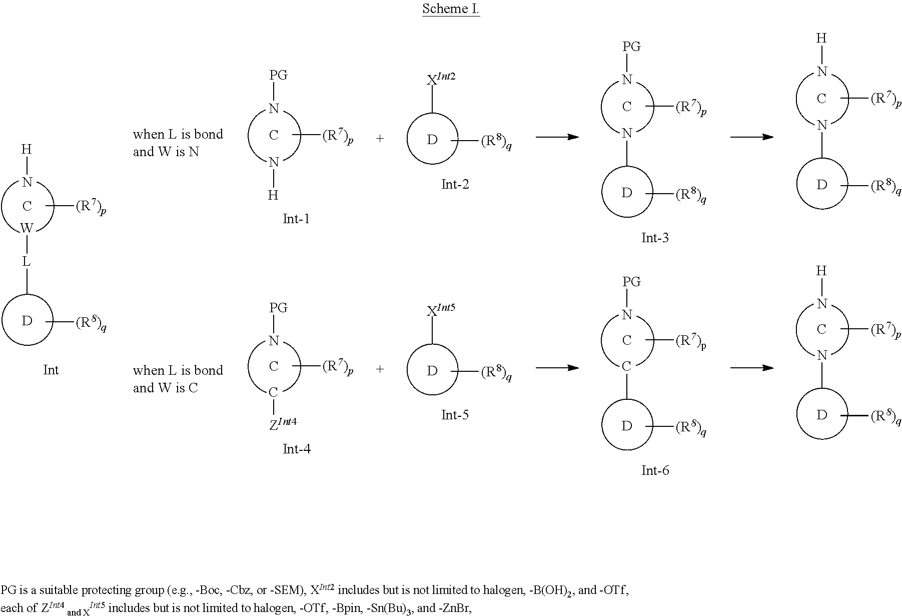

Intermediates of Formula Int can also be prepared according to the procedures shown in Scheme Ia. Reacting of Int-7 (wherein PG is a suitable protecting group such as Boc; LG is a suitable leaving group such as Br, OMs or OH) with Int-8 (wherein L is a suitable linking group such as OH or NH₂) under suitable conditions, such as nucleophilic substitution conditions or Mitsunobu reaction conditions, can give compounds of Formula Int-9. Removal of protecting group in Int-9 can afford compounds of Formula Int.

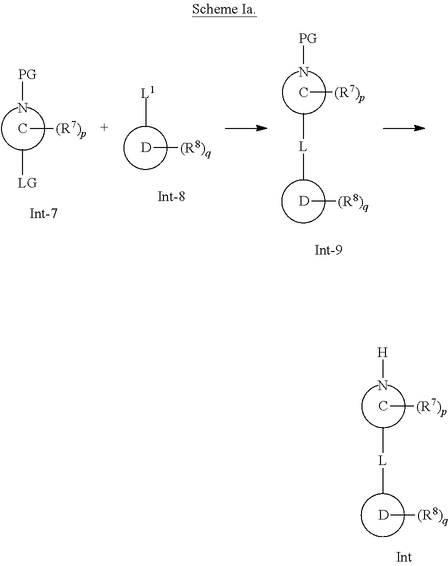

Compounds of Formula I-A can be prepared, for example, according to the procedures shown in Scheme II. Reacting compound II-1 and compound II-2 under reductive amination conditions in the presence of an appropriate reagent (e.g., a reducing agent such as NaBH(OAc)₃ or NaBH₃CN) followed by cyclization affords compound II-3, which can be converted to compound II-4 under suitable conditions (e.g., treating with triphosgene followed by NH₄OH). Treating compound II-4 under suitable conditions (e.g., palladium-catalyzed cross-coupling) gives compound 11-5. Compound II-6 is then prepared by treating compound II-5 under suitable conditions (e.g., Suzuki, Stille, or Negishi coupling). Finally, treating compound 11-6 with Int affords compound of Formula I-A.

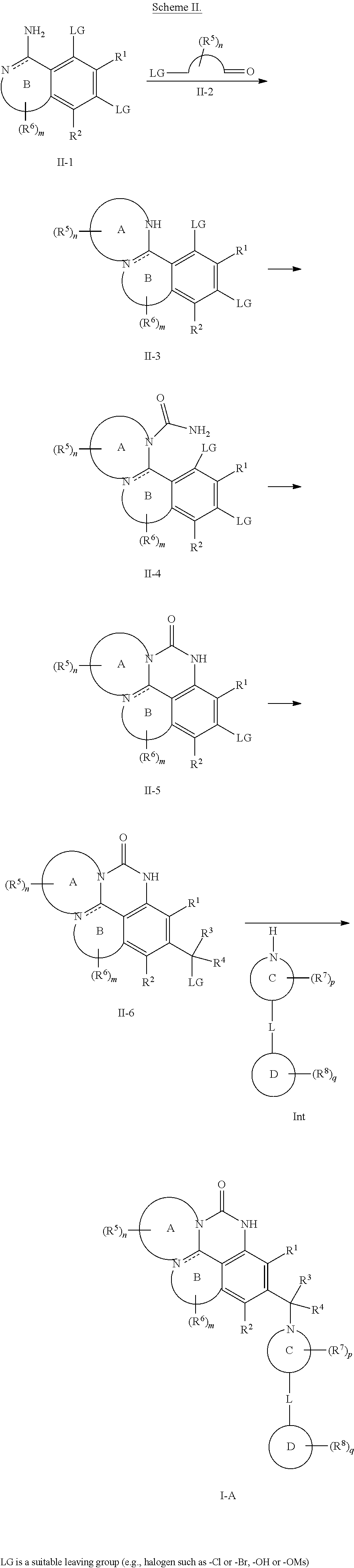

Compounds of Formula I-B can be prepared, for example, according to the procedures shown in Scheme III. Treating compound III-1 with compound III-2 under suitable conditions (e.g., nucleophilic aromatic substitution, or Buchwald-Hartwig cross-coupling) affords compound III-3 which can be converted to compound III-4 under suitable conditions (e.g., palladium-catalyzed cross-coupling). Compound III-4 can be subsequently converted into compound III-5 under suitable conditions (e.g., treating with NaH followed by 4-methoxybenzyl isocyanate). Compound III-6 is then prepared by treating compound III-5 under suitable conditions (e.g., Suzuki, Stille, or Negishi coupling). Finally, treating compound III-6 with Int affords compound of Formula I-B.

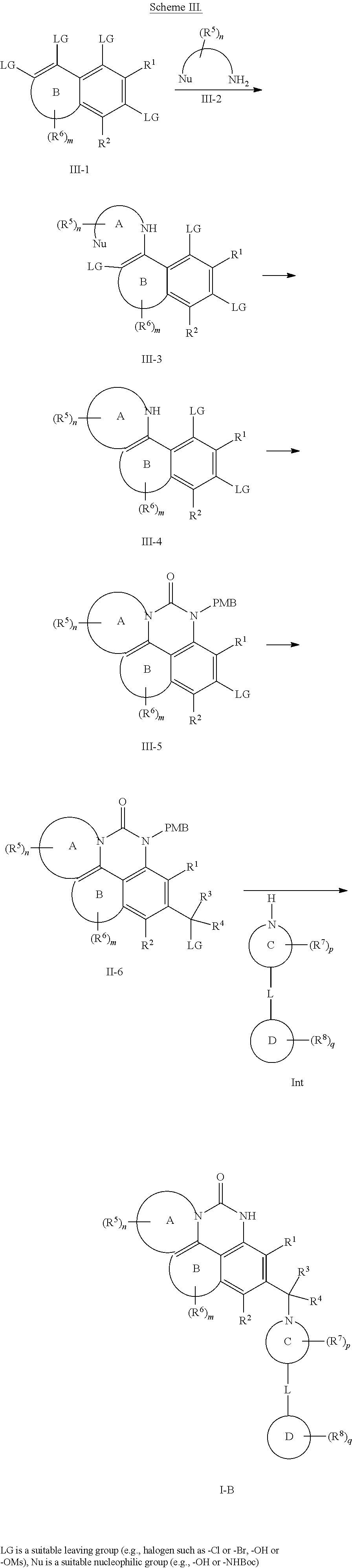

Compounds of Formula I-C can be prepared, for example, according to the procedures shown in Scheme IV. Compound IV-3 is prepared by treating compound IV-2 with an appropriate reagent (e.g., 9-BBN) followed by cross-coupling with compound IV-1 under suitable conditions (e.g., PdCl₂(dppf)/K₃PO₄). Subsequently, compound IV-3 is converted to compound IV-4 via removing Boc group followed by ring closing under suitable conditions (e.g., nucleophilic aromatic substitution, or Buchwald-Hartwig cross-coupling). Under suitable conditions (e.g., treating with NaH followed by 4-methoxybenzyl isocyanate), compound IV-4 can be converted into compound IV-5. Compound IV-6 is then prepared by treating compound IV-5 under suitable conditions (e.g., Suzuki, Stille, or Negishi coupling). Finally, treating compound IV-6 with Int affords compound of Formula I-C.

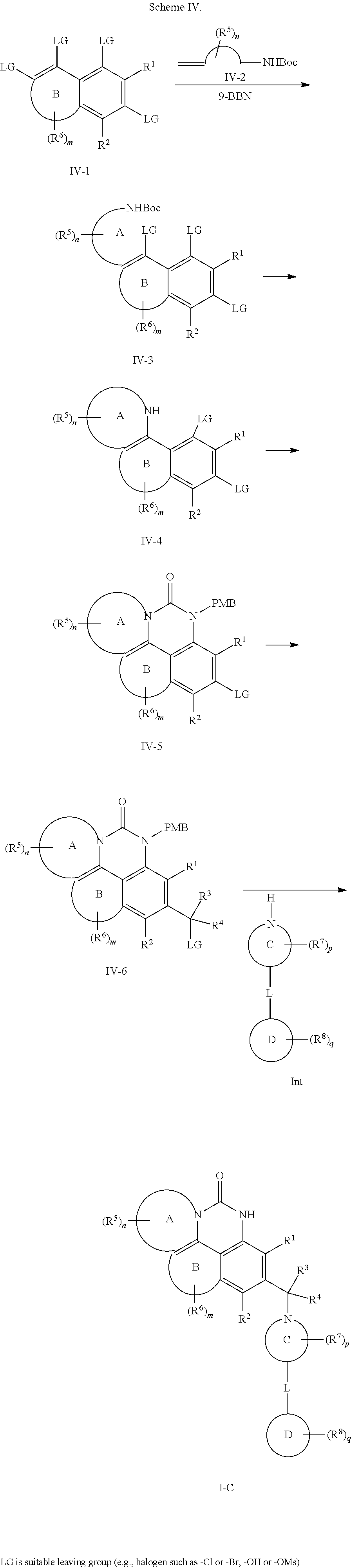

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.”, as used herein, are understood in the art, and refer generally to a temperature, e.g. a reaction temperature, that is about the temperature of the room in which the reaction is carried out, for example, a temperature from about 20° C. to about 30° C.

### Methods of Use

The present disclosure provides uses for compounds and compositions described herein. In some embodiments, provided compounds and compositions are for use in medicine (e.g., as therapy). In some embodiments, provided compounds and compositions are useful in treating a disease, disorder, or condition, wherein an underlying pathology is, wholly or partially, mediated by PARP1. In some embodiments, provided compounds and compositions are useful in research as, for example, analytical tools and/or control compounds in biological assays.

In some embodiments, the present disclosure provides methods of administering provided compounds or compositions to a subject in need thereof. In some embodiments, the present disclosure provides methods of administering provided compounds or compositions to a subject suffering from or susceptible to a disease, disorder, or condition associated with PARP1. In some embodiments, the present disclosure provides methods of administering provided compounds or compositions to a subject suffering from or susceptible to a disease, disorder, or condition, wherein an underlying pathology is, wholly or partially, mediated by PARP1.

In some embodiments, the compounds provided herein (e.g., a compound of Formula I, or a pharmaceutically acceptable salt thereof) are useful as PARP1 inhibitors. In some embodiments, the present disclosure provides methods of inhibiting PARP1 in a subject comprising administering a provided compound or composition. In some embodiments, the present disclosure provides methods of inhibiting PARP1 in a biological sample comprising contacting the sample with a provided compound or composition.

In some embodiments, the present disclosure provides methods of treating a disease, disorder or condition associated with PARP1 in a subject in need thereof, comprising administering to the subject a provided compound or composition. In some embodiments, a disease, disorder or condition is associated with overexpression of PARP1. In some embodiments, the present disclosure provides methods of treating a disease, disorder or condition, wherein an underlying pathology is, wholly or partially, mediated by PARP1, in a subject in need thereof, comprising administering to the subject a provided compound or composition.

In some embodiments, the present disclosure provides methods of treating cancer, comprising administering a compound, salt, or composition provided herein to a subject in need thereof. In some embodiments, the present disclosure provides methods of treating proliferative diseases, comprising administering a compound, salt, or composition provided herein to a subject in need thereof. In some embodiments, the present disclosure provides methods of treating metastatic cancers, comprising administering a compound, salt, or composition provided herein to a subject in need thereof. Exemplary cancers include but are not limited to breast cancer, ovarian cancer, cervical cancer, epithelial ovarian cancer, fallopian tube cancer, primary peritoneal cancer, endometrial cancer, prostate cancer, testicular cancer, pancreatic cancer, esophageal cancer, head and neck cancer, gastric cancer, bladder cancer, lung cancer (e.g., adenocarcinoma, non-small-cell lung carcinoma (NSCLC) and small-cell lung carcinoma (SCLC)), bone cancer (e.g., osteosarcoma), colon cancer, rectal cancer, thyroid cancer, brain and central nervous system cancers, glioblastoma, neuroblastoma, neuroendocrine cancer, rhabdoid cancer, keratoacanthoma, epidermoid carcinoma, seminoma, melanoma, sarcoma (e.g., liposarcoma), bladder cancer, uterine serous carcinoma, liver cancer (e.g., hepatocellular carcinoma), kidney cancer (e.g., renal cell carcinoma), myeloid disorders (e.g., acute myelogenous leukemia (AML), chronic myelogenous leukemia (CML), myelodysplastic syndrome and promyelocytic leukemia), and lymphoid disorders (e.g., leukemia, multiple myeloma, mantle cell lymphoma, acute lymphocytic leukemia (ALL), chronic lymphocytic leukemia (CLL), B-cell lymphoma, T-cell lymphoma, Hodgkin's lymphoma, and non-Hodgkin's lymphoma, hairy cell lymphoma).

In some embodiments, the cancer is selected from ovarian cancer, breast cancer, prostate cancer, and pancreatic cancer. In some embodiments, the cancer is ovarian cancer. In some embodiments, the cancer is breast cancer. In some embodiments, the cancer is prostate cancer. In some embodiments, the cancer is pancreatic cancer.

When used as a single agent for monotherapy, the compounds, salts, and compositions provided herein are expected to selectively kill tumor cells characterized by homologous recombination deficiency while generating minimal impact on normal tissues. In some embodiments, the present disclosure provides methods of treating advanced cancer induced by or correlated with a dysregulated DNA repair system, comprising administering a provided compound or composition to a subject in need thereof. In some embodiments, such advanced cancers include but are not limited to breast cancer, ovarian cancer, pancreatic cancer, and prostate cancer. These malignant tumors are features of deleterious or suspected deleterious mutations of key genes involved in DNA damage repair pathways. In some embodiments, such key genes include but are not limited to ATM, ATR, BAP1, BRCA1, BRCA2, CDK12, CHEK2, FANCA, FANCC, FANCD2, FANCE, FANCF, PALB2, NBS1, WRN, RAD51C, RAD51D, MRE11A, CHEK1, BLM, RAD51B, and BRIP1. Cancer patients with such mutations can be identified using companion diagnostics. Advanced cancer patients with a positive status of homologous recombination deficiency are expected to benefit from monotherapy with a compound, salt, or composition provided herein.

When used as a frontline maintenance therapy, the compounds, salts, or compositions provided herein are useful in treating cancer featured by dysregulated DNA damage repair. Exemplary cancers include but are not limited to triple-negative breast cancer, high-grade serous ovarian cancer, platinum-sensitive advanced pancreatic cancer, and castration-resistant prostate cancer. These tumors are typically sensitive to platinum-based therapies and other DNA damaging agents. As a maintenance therapy, provided compounds and compositions of the present disclosure may reduce risks of recurrence or relapse and therefore prolong progression free survival of patients with advanced cancers.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” PARP1 with a compound described herein includes the administration of a compound described herein to an individual or patient, such as a human, having PARP1, as well as, for example, introducing a compound described herein into a sample containing a cellular or purified preparation containing the PARP1.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapy

Cancer cell growth and survival can be impacted by dysfunction in multiple signaling pathways. It is useful to combine compounds modulating different biological targets to treat such conditions. Targeting more than one signaling pathway or more than one biological molecule involved in a given signaling pathway also may reduce the likelihood of drug resistance.

In some embodiments, a compound, salt, or composition provided herein is administered as part of a combination therapy. As used herein, the term “combination therapy” refers to those situations in which a subject is simultaneously exposed to two or more therapeutic or prophylactic regimens (e.g., two or more therapeutic or prophylactic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

For example, in some embodiments, a compound, salt, or composition provided herein is administered to a subject who is receiving or has received one or more additional therapies (e.g., an anti-cancer therapy and/or therapy to address one or more side effects of such anti-cancer therapy, or otherwise to provide palliative care).

Exemplary additional therapies include but are not limited to chemotherapies, radiotherapies, anti-inflammatory agents, steroids, immunosuppressants, immune-oncology agents, metabolic enzyme inhibitors, chemokine receptor inhibitors, phosphatase inhibitors, and targeted therapies such as kinase inhibitors.

In some embodiments, a compound, salt, or composition provided herein can be combined with one or more agents targeting the following biological targets, including but not limiting to Wee1, ATR, ATM, DNA-PK, CDK4/6, CHK1/2, HER2, PI3K, mTOR, EGFR, VEGFR, FGFR, PDGFR, BTK, IGF-1R, BRAF, MEK, KRAS, EZH2, BCL2, HSP90, HDAC, Topoisomerases, HIF-2a, androgen receptor, estrogen receptor, proteosome, RAD51, RAD52, POLQ, WRN, PD-1, and PD-L1. Hypoxia induced by HIF-2a inhibition results in down-regulated expression of the BRCA gene, consequently making tumor cells more vulnerable to PARP1 inhibition. Exemplary cancers for combination of PARP1 and HIF-2a inhibitors include but not limited to clear cell renal cell carcinoma, particularly for the subgroup with the tumor suppressor von Hippel Lindau (VHL) deficiency.

In some embodiments, a compound, salt, or composition provided herein can be combined with chemotherapies for treatment of cancer. In some embodiments, a compound, salt, or composition provided herein can be combined with chemotherapies for treatment of high-grade serous ovarian cancer. Exemplary chemotherapies include but are not limited to platinum-based therapy, taxane-based therapy and some others including albumin bound paclitaxel, altretamine, capecitabine, cyclophosphamide, gemcitabine, ifosfamide, irinotecan, liposomal doxorubicin, melphalan, pemetrexed, topotecan, and vinorelbine.

In some embodiments, a compound, salt, or composition provided herein can be combined with chemotherapies for treatment of advanced metastatic breast cancer. Exemplary chemotherapies include but are not limited to taxanes such as paclitaxel, docetaxel, and albumin-bound paclitaxel, anthracyclines, platinum agents, vinorelbine, capecitabine, gemcitabine, ixabepilone, and eribulin. In some embodiments, such combination therapies can be used for malignancies derived from other histologies, including but limited to brain, lung, kidney, liver, and hematologic cancers.

Radiotherapies are widely used in clinic for treatment of cancers. A compound, salt, or composition provided herein may improve the effectiveness of radiation therapy through its potent activity in suppressing DNA damage repair. In some embodiments, a compound, salt, or composition provided herein can be combined with radiotherapies for treatment of cancer. Exemplary cancers that can be treated with radiotherapies include but are not limited to small cell lung cancer, leukemias, lymphomas, germ cell tumors, non-melanoma skin cancer, head and neck cancer, breast cancer, non-small cell lung cancer, cervical cancer, anal cancer, and prostate cancer. In some embodiments, a compound, salt, or composition provided herein may overcome the resistance of certain cancer to radiotherapy, particularly for renal cell carcinoma and melanomas.

Immunotherapies including antibodies of PD1, PD-L1, and CTLA4 have been successfully used for treatment of cancer. Despite this huge success, resistance and relapse remain a challenge for the vast majority of cancer patients. In some embodiments, a compound, salt, or composition provided herein can be combined with immunotherapies to improve the effectiveness of conventional antibody-medicated immunotherapies by promoting DNA damage, increasing mutation burden, and modulating the STING innate immune pathway. In some embodiments, a compound, salt, or composition provided herein can be combined with immunotherapies for treatment of adult and pediatric patients with unresectable or metastatic tumors. In some embodiments, a compound, salt, or composition provided herein can be combined with immunotherapies for treatment of cancer. Exemplary cancers include but are not limited to non-small cell lung cancer, melanoma, head and neck squamous cell carcinoma, classical Hodgkin lymphoma, urothelial carcinoma, microsatellite instability-high cancer, gastric cancer, cervical cancer, primary mediastinal large B-cell lymphoma, hepatocellular carcinoma, Merkel cell carcinoma, renal cell carcinoma, esophageal cancer, endometrial cancer, tumor mutational burden-high cancer, cutaneous squamous cell carcinoma, microsatellite instability-high or mismatch repair deficient colorectal cancer, and triple-negative breast cancer.

In some embodiments, a compound, salt, or composition provided herein can be combined with targeted therapies of well-established therapeutic targets including but not limited to PI3K inhibitors, KRAS inhibitors, CDK4/6 inhibitors, BRAF inhibitors, MEK inhibitors, androgen receptor inhibitors, selective estrogen receptor modulators, proteosome inhibitors, mTOR inhibitors, EGFR inhibitors, FGFR inhibitors, MET inhibitors, PDGFR inhibitors, VEGFR inhibitors, EZH2 inhibitors, BTK inhibitors, and BCL2 inhibitors for treatment of cancer. Exemplary cancers include but are not limited to breast cancer, ovarian cancer, non-small cell lung cancer, hepatocellular carcinoma, clear cell renal cell carcinoma, melanoma, colorectal cancer, bladder cancer, prostate cancer, cholangiocarcinoma, and hematologic cancers.

In some embodiments, a compound, salt, or composition provided herein can be combined with inhibitors of other DNA damage repair proteins including but not limited to CHEK1, CHEK2, ATM, ATR, DNA-PK, WEE1, RAD51, RAD52, POLQ, and WRN for treatment of cancer sensitive to DNA damage. In some embodiments, a compound, salt, or composition provided herein can be combined with a WEE1 inhibitor for treatment of uterine serous carcinoma and cancers with mutation of the TP53 genes. In some embodiments, a compound, salt, or composition provided herein can be combined with a WRN inhibitor for treatment of microsatellite instability-high cancers, such as colon cancer, gastric cancer, endometrium cancer, ovarian cancer, hepatobiliary tract cancer, urinary tract cancer, brain cancer, and skin cancers.

### Pharmaceutical Formulations and Dosage Forms

When employed as pharmaceuticals, the compounds and salts provided herein can be administered in the form of pharmaceutical compositions which refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

The liquid forms in which the compounds and compositions of the present invention can be incorporated for administration orally or by injection include aqueous solutions, suitably flavored syrups, aqueous or oil suspensions, and flavored emulsions with edible oils.

The compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are provided herein.

### Labeled Compounds and Assay Methods

Another aspect of the present invention relates to fluorescent dye, spin label, heavy metal or radio-labeled compounds of the invention that would be useful not only in imaging but also in assays, both in vitro and in vivo, for localizing and quantitating the PARP1 protein in tissue samples, including human, and for identifying PARP1 protein ligands by inhibition binding of a labeled compound. Accordingly, the present invention includes PARP1 biochemical assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro PARP1 labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

One or more constituent atoms of the compounds presented herein can be replaced or substituted with isotopes of the atoms in natural or non-natural abundance. In some embodiments, one or more atoms are replaced or substituted by deuterium. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ being substituted for —CH₃). In some embodiments, alkyl groups of the disclosed Formulas (e.g., the compound of any of Formulas I-IV) can be perdeuterated.

In some embodiments, the compound provided herein (e.g., the compound of any of Formulas I-IV), or a pharmaceutically acceptable salt thereof, comprises at least one deuterium atom.

In some embodiments, the compound provided herein (e.g., the compound of any of Formulas I-IV), or a pharmaceutically acceptable salt thereof, comprises two or more deuterium atoms.

In some embodiments, the compound provided herein (e.g., the compound of any of Formulas I-IV), or a pharmaceutically acceptable salt thereof, comprises three or more deuterium atoms.

In some embodiments, for a compound provided herein (e.g., the compound of any of Formulas I-IV), or a pharmaceutically acceptable salt thereof, all of the hydrogen atoms are replaced by deuterium atoms (i.e., the compound is “perdeuterated”).

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ⁸²Br.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the PARP1 protein. Accordingly, the ability of a test compound to compete with the radio-labeled compound for binding to the PARP1 protein directly correlates to its binding affinity.

### Kits

The present invention also includes pharmaceutical kits useful, for example, in the treatment or prevention of PARP1-associated diseases or disorders referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples were found to be inhibitors of PARP1 as described below.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

The final compounds were purified on a preparative scale reverse-phase high performance liquid chromatography (RP-HPLC) or flash chromatography (silica gel) as indicated in the Examples. Typical preparative reverse-phase high performance liquid chromatography (RP-HPLC) column conditions are as follows:

TFA conditions: column, Waters XSelect CSH C18 5 μm particle size, 30×150 mm; eluting with mobile phase A: water (0.05% trifluoroacetic acid), mobile Phase B: acetonitrile; the flow rate, 60 mL/min.

NH₄HCO₃ conditions: column, waters XBridge BEH C18 5 μm particle size, 30×150 mm; eluting with mobile phase A: water (10 mM ammonium bicarbonate), mobile Phase B: acetonitrile; the flow rate, 60 mL/min.

HCOOH conditions: column, Sunfire Prep C18 OBD 5 μm particle size, 30×150 mm; eluting with mobile phase A: water (0.1% formic acid), mobile Phase B: acetonitrile; the flow rate, 60 mL/min.

The separating gradient was optimized for each compound. The separated compounds were typically subjected to analytical liquid chromatography mass spectrometry (LCMS) for purity check under the following conditions: Instrument: Shimadzu LCMS-2020, column: Halo C₁₈ 2 μm particle size, 3×30 mm; buffers: mobile phase A: 0.05% TFA in water and mobile phase B: acetonitrile; gradient: 0 to 60% of B in 1.9 min, 60% to 100% of B in 0.35 min with flow rate 1.5 mL/min.

### Intermediate 1. N,6-Dimethyl-5-(piperazin-1-yl)picolinamide

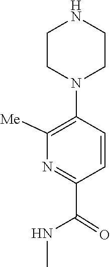 [SMILES: CNC(=O)c1ccc(N2CCNCC2)c(C)n1]

### Step 1: 5-Bromo-N,6-dimethylpicolinamide

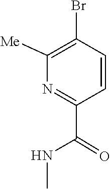

A mixture of 5-bromo-6-methylpyridine-2-carboxylic acid (5 g, 23.1 mmol) was treated with O-(7-azabenzotriazol-1-yl)-N,N,N,N-tetramethyl uronium hexafluorophosphate (10.56 g, 27.8 mmol) in N,N-dimethylformamide (80 mL) at room temperature for 30 min, followed by the addition of N-ethyl-N-isopropylpropan-2-amine (14.96 g, 115.7 mmol) and methylamine hydrochloride (2.34 g, 34.7 mmol). The resulting mixture was stirred at the same temperature for 2 h, and then diluted with ethyl acetate (500 mL). The resulting mixture was washed with water (3×100 mL) and brine (3×100 mL). The combined organics were dried with anhydrous sodium sulfate. After filtration, the filtrate was concentrated under reduced pressure. The residue was purified by silica gel column chromatography, eluted with 50% ethyl acetate in petroleum ether to provide the desired product as a white solid (4.53 g, 85%). LCMS calculated for C₈H₁₀BrN₂O (M+H)⁺ m/z=229.0; found 228.9; ¹H NMR (300 MHz, CD₃OD) δ 8.11 (d, J=8.1 Hz, 1H), 7.81 (d, J=8.1 Hz, 1H), 2.97 (s, 3H), 2.72 (s, 3H).

### Step 2: tert-Butyl 4-(2-methyl-6-(methylcarbamoyl)pyridin-3-yl)piperazine-1-carboxylate

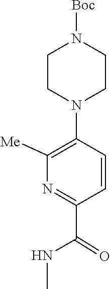 [SMILES: CNC(=O)c1ccc(N2CCN(C(=O)OC(C)(C)C)CC2)c(C)n1]

A mixture of 5-bromo-N,6-dimethylpyridine-2-carboxamide (700 mg, 3.1 mmol), tert-butyl piperazine-1-carboxylate (854 mg, 4.6 mmol), palladium (II) acetate (69 mg, 0.31 mmol), racemic-2,2′-bis(diphenylphosphino)-1,1′-binaphthyl (285 mg, 0.46 mmol), and cesium carbonate (1.99 g, 6.1 mmol) in toluene (12 mL) was stirred at 80° C. for 18 h under nitrogen atmosphere. After cooling to room temperature, the resulting mixture was concentrated under reduced pressure, and the residue was purified by silica gel column chromatography, eluted with 60% ethyl acetate in petroleum ether to afford the desired product as a yellow solid (800 mg, 78%). LCMS calculated for C₁₇H₂₇N₄O₃ (M+H)⁺ m/z=335.2; found 335.3; ¹H NMR (300 MHz, CDCl₃) δ 8.12 (brs, 1H), 8.04 (d, J=8.4 Hz, 1H), 7.38 (d, J=8.4 Hz, 1H), 3.64-3.61 (m, 4H), 3.04 (d, J=5.1 Hz, 3H), 2.95-2.92 (m, 4H), 2.58 (s, 3H), 1.51 (s, 9H).

Step 3: N,6-Dimethyl-5-(piperazin-1-yl)picolinamide A mixture of tert-butyl 4-[2-methyl-6-(methylcarbamoyl)pyridin-3-yl]piperazine-1-carboxylate (150 mg, 0.45 mmol) in dichloromethane (6 mL) was treated with hydrogen chloride (4 mL, 4 M in dioxane). After stirring at room temperature for 2 h, the mixture was concentrated under reduced pressure. To the residue was charged a saturated sodium bicarbonate aqueous solution (30 mL). The mixture was extracted with dichloromethane (4×50 mL). The combined organic layers were dried with anhydrous sodium sulfate. After filtered, the filtrate was concentrated under reduced pressure to provide the desired product which was used in the next step without further purification. LCMS calculated for C₁₂H₁₉N₄O (M+H)⁺ m/z=235.2; found 235.1.

### Intermediate 2. N-Cyclopropyl-6-methyl-5-(piperazin-1-yl)picolinamide

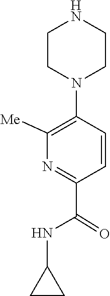 [SMILES: Cc1nc(C(=O)NC2CC2)ccc1N1CCNCC1]

### Step 1: 5-Bromo-N-cyclopropyl-6-methylpicolinamide

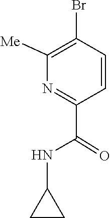 [SMILES: Cc1nc(C(=O)NC2CC2)ccc1Br]

To a solution of 5-bromo-6-methylpicolinic acid (2 g, 9.26 mmol) in N,N-dimethylformamide (30 mL) was added 2-(7-azabenzotriazol-1-yl)-N,N,N,N-tetramethyluronium hexafluorophosphate (4.22 g, 11.11 mmol). The mixture was stirred at room temperature for 30 min. N-Ethyl-N-isopropylpropan-2-amine (5.98 g, 46.29 mmol) was added followed by cyclopropanamine (0.79 g, 13.89 mmol). After stirring at room temperature for 2 h, the reaction was diluted with water (300 mL) and extracted with ethyl acetate (3×150 mL). The combined organic layers were washed with brine (800 mL), dried over anhydrous sodium sulfate. After filtration, the filtrate was concentrated. The residue was purified by silica gel column chromatography, eluted with 30% ethyl acetate in petroleum ether to give the desired product as a yellow oil (1.83 g, 77%). LCMS calculated for C₁₀H₁₂BrN₂O (M+H)⁺ m/z=255.0; found 255.0.

### Step 2: tert-Butyl 4-(6-(cyclopropylcarbamoyl)-2-methylpyridin-3-yl)piperazine-1-carboxylate

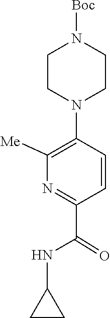 [SMILES: Cc1nc(C(=O)NC2CC2)ccc1N1CCN(C(=O)OC(C)(C)C)CC1]

To a round bottom flask equipped with a magnetic stir bar was added 5-bromo-N-cyclopropyl-6-methylpicolinamide (1.83 g, 7.17 mmol), cesium carbonate (4.67 g, 14.35 mmol), racemic-2,2′-bis(diphenylphosphino)-1,1′-binaphthyl (0.67 g, 1.07 mmol), palladium acetate (0.16 g, 0.72 mmol) and tert-butyl piperazine-1-carboxylate (2 g, 10.76 mmol). The flask was sealed with a rubber septum, evacuated, and backfilled nitrogen (this process was repeated a total of three times). Toluene (20 mL) was added and the reaction mixture was heated at 80° C. for 16 h. After cooling to room temperature, the mixture was concentrated. The residue was purified by silica gel column chromatography, eluted with 30% ethyl acetate in petroleum ether to give the desired product as a brown yellow oil (2.25 g, 87%). LCMS calculated for C₁₉H₂₉N₄O₃ (M+H)⁺ m/z=361.2; found 361.2; ¹H NMR (400 MHz, CDCl₃) δ 7.99 (d, J=8.4 Hz, 1H), 7.32 (d, J=8.4 Hz, 1H), 3.61-3.59 (m, 4H), 2.91-2.89 (m, 5H), 2.52 (s, 3H), 1.49 (s, 9H), 0.89-0.84 (m, 2H), 0.68-0.64 (m, 2H).

Step 3: N-cyclopropyl-6-methyl-5-(piperazin-1-yl)picolinamide A mixture of tert-butyl 4-(6-(cyclopropylcarbamoyl)-2-methylpyridin-3-yl)piperazine-1-carboxylate (2.25 g, 6.24 mmol) in hydrogen chloride (4 M in 1,4-dioxane, 30 mL) was stirred at room temperature for 1 h. The resulting mixture was concentrated. The residue was treated with saturated aqueous sodium bicarbonate (100 mL) and extracted with dichloromethane (5×80 mL). The combined organic layers were dried over anhydrous sodium sulfate and filtered. The filtrate was concentrated under reduced pressure. The residue was purified by silica gel column chromatography, eluted with 10% ammonia (7 N in methanol) in dichloromethane to give the desired product as a light-yellow solid (1.58 g, 97%). LCMS calculated for C₁₄H₂₁N₄O (M+H)⁺ m/z=261.2; found 261.2.

### Intermediate 3. Methyl 6-methyl-5-(piperazin-1-yl)picolinate

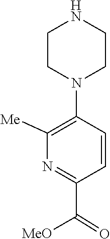 [SMILES: COC(=O)c1ccc(N2CCNCC2)c(C)n1]

### Step 1: Methyl 5-bromo-6-methylpicolinate

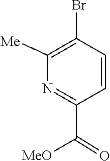 [SMILES: COC(=O)c1ccc(Br)c(C)n1]

To a stirred mixture of 5-bromo-6-methylpicolinic acid (5 g, 23.15 mmol) in dichloromethane (40 mL) was added N,N-dimethylpyridin-4-amine (4.24 g, 34.71 mmol) at 0° C. 1-Ethyl-3-(3-dimethylaminopropyl)carbodiimide HCl (4.67 g, 24.36 mmol) was added in portions. Then methanol (10 mL, 246.96 mmol) was added. The resulting mixture was stirred at room temperature for 16 h. The reaction mixture was diluted with water (100 mL) and extracted with ethyl acetate (3×100 mL). The combined organic layers were dried over anhydrous sodium sulfate. After filtration, the filtrate was concentrated under reduced pressure. The residue was purified by silica gel column chromatography, eluted with 30% ethyl acetate in petroleum ether to give the desired product as a white solid (3.7 g, 69%). LCMS calculated for C₈H₉BrNO₂ (M+H)⁺ m/z=230.0; found 229.9; ¹H NMR (300 MHz, CDCl₃) δ 7.97 (d, J=8.2 Hz, 1H), 7.83 (d, J=8.2 Hz, 1H), 4.00 (s, 3H), 2.77 (s, 3H).

### Step 2: tert-Butyl 4-(6-(methoxycarbonyl)-2-methylpyridin-3-yl)piperazine-1-carboxylate

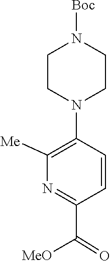 [SMILES: COC(=O)c1ccc(N2CCN(C(=O)OC(C)(C)C)CC2)c(C)n1]

To a round bottom flask equipped with a magnetic stir bar was added methyl 5-bromo-6-methylpicolinate (3.6 g, 15.65 mmol), cesium carbonate (15.30 g, 46.94 mmol), 2-dicyclohexylphosphino-2′,6′-diisopropoxybiphenyl (1.46 g, 3.13 mmol), tris(dibenzylideneacetone)dipalladium (1.43 g, 1.57 mmol) and tert-butyl piperazine-1-carboxylate (3.21 g, 17.21 mmol). The flask was sealed with a rubber septum, evacuated and backfilled with nitrogen (this process was repeated a total of three times). Toluene (100 mL) was added. The reaction mixture was stirred at 100° C. for 3 h. After cooling to room temperature, the mixture was concentrated. The residue was purified by silica gel column chromatography, eluted with 50% ethyl acetate in petroleum ether to give the desired product as a brown solid (5 g, 96%). LCMS calculated for C₁₇H₂₆N₃O₄ (M+H)⁺ m/z=336.2; found 336.1; ¹H NMR (400 MHz, CDCl₃) δ 7.99 (d, J=8.0 Hz, 1H), 7.33 (d, J=8.4 Hz, 1H), 3.99 (s, 3H), 3.63-3.61 (m, 4H), 2.96-2.94 (m, 4H), 2.67 (s, 3H), 1.49 (s, 9H).

### Step 3: Methyl 6-methyl-5-(piperazin-1-yl)picolinate

The solution of tert-butyl 4-(6-(methoxycarbonyl)-2-methylpyridin-3-yl)piperazine-1-carboxylate (5 g, 14.91 mmol) in hydrogen chloride (4 M in 1,4-dioxane, 50 mL) was stirred at room temperature for 1 h. The mixture was neutralized with saturated aqueous sodium bicarbonate. The resulting mixture was extracted with 25% isopropanol in chloroform(3×200 mL). The combined organic layers were washed with brine (2×300 mL), dried over anhydrous sodium sulfate. After filtration, the filtrate was concentrated under reduced pressure. The residue was purified by silica gel column chromatography, eluted with 10% methanol in dichloromethane to give the desired product as a brown oil (3.3 g, 94%). LCMS calculated for C₁₂H₁₈N₃O₂ (M+H)⁺ m/z=236.1; found 236.1; ¹H NMR (400 MHz, CDCl₃) δ 7.97 (d, J=8.4 Hz, 1H), 7.31 (d, J=8.4 Hz, 1H), 3.98 (s, 3H), 3.09-3.06 (m, 4H), 2.99-2.96 (m, 4H), 2.62 (s, 3H).

### Example 1. 5-(4-((7-Fluoro-9-oxo-2,3,8,9-tetrahydro-1H-3a,4,8,9a-tetraazacyclopenta[def]phenanthren-6-yl)methyl)piperazin-1-yl)-N,6-dimethylpicolinamide

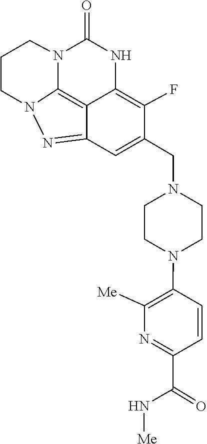 [SMILES: CNC(=O)c1ccc(N2CCN(Cc3cc4nn5c6c4c(c3F)NC(=O)N6CCC5)CC2)c(C)n1]

### Step 1: 2-Bromo-4-chloro-3,6-difluorobenzonitrile

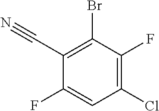 [SMILES: N#Cc1c(F)cc(Cl)c(F)c1Br]

A mixture of 4-chloro-2,5-difluorobenzonitrile (6 g, 34.57 mmol), N-bromosuccinimide (6.77 g, 38.03 mmol), palladium (II) acetate (0.78 g, 3.46 mmol) and p-toluenesulfonic acid (3.29 g, 17.29 mmol) in 1,2-dichloroethane (60 mL) was stirred at 70° C. for 12 h under nitrogen atmosphere. Upon cooling to room temperature, the reaction was concentrated under reduced pressure. The residue was purified by silica gel column chromatography, eluted with 50% ethyl acetate in petroleum ether to provide the desired product as a white solid (2.2 g, 25%). ¹H NMR (300 MHz, CDCl₃) δ 7.36 (dd, J=7.8, 5.4 Hz, 1H).

### Step 2: 4-Bromo-6-chloro-5-fluoro-1H-indazol-3-amine

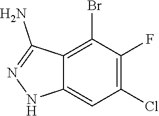 [SMILES: Nc1n[nH]c2cc(Cl)c(F)c(Br)c12]

To a mixture of 2-bromo-4-chloro-3,6-difluorobenzonitrile (7 g, 27.7 mmol) in tert-butanol (70 mL) was added hydrazine hydrate (80% solution in water, 8.67 g, 138.7 mmol) dropwise at 0° C. The resulting mixture was stirred at 100° C. for 16 h. Upon cooling to room temperature, the mixture was concentrated under reduced pressure. The residue was triturated with ethyl acetate and filtered. The solid was dried under vacuum to afford the desired product as a white solid (6.6 g, 91%). LCMS calculated for C₇H₃BrClFN₃ (M−H)⁻ m/z=261.9; found 261.8.

### Step 3: 10-Bromo-8-chloro-9-fluoro-1,2,3,4-tetrahydropyrimido[1,2-b]indazole

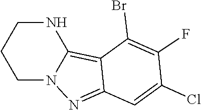 [SMILES: Fc1c(Cl)cc2nn3c(c2c1Br)NCCC3]

To a mixture of 4-bromo-6-chloro-5-fluoro-1H-indazol-3-amine (2 g, 7.56 mmol) in methanol (30 mL) were added 3-((tert-butyldimethylsilyl)oxy)propanal (2.85 g, 15.12 mmol) and acetic acid (0.91 g, 15.12 mmol) at room temperature. The resulting mixture was stirred at 50° C. for 5 h, and then cooled to room temperature. Sodium cyanoborohydride (1.43 g, 22.69 mmol) was added in portions. The resulting mixture was stirred at 50° C. for additional 2 h. Upon cooling to room temperature, the mixture was basified to pH=10 with ammonia (7 M in methanol solution). The resulting mixture was concentrated under reduced pressure. The residue was purified by silica gel column chromatography, eluted with 30% ethyl acetate in dichloromethane to afford the desired product as a yellow solid (400 mg, 17%). LCMS calculated for C₁₀H₉BrClFN₃ (M+H)⁺ m/z=304.0; found 303.9; ¹H NMR (400 MHz, CDCl₃) δ 7.40 (d, J=6.0 Hz, 1H), 5.33 (s, 1H), 4.36 (t, J=6.0 Hz, 2H), 3.54 (t, J=5.6 Hz, 2H), 2.31-2.25 (m, 2H).

### Step 4: 10-Bromo-8-chloro-9-fluoro-3,4-dihydropyrimido[1,2-b]indazole-1(2H)-carboxamide

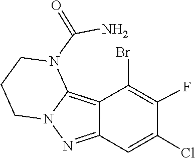 [SMILES: NC(=O)N1CCCn2nc3cc(Cl)c(F)c(Br)c3c21]

To a mixture of 10-bromo-8-chloro-9-fluoro-1,2,3,4-tetrahydropyrimido[1,2-b]indazole (400 mg, 1.31 mmol) and N,N-dimethylpyridin-4-amine (16.05 mg, 0.13 mmol) in tetrahydrofuran (15 mL) were added triethylamine (2.66 g, 26.26 mmol) and triphosgene (1.95 g, 6.57 mmol) in portions at 0° C. The resulting mixture was stirred for 2 h at room temperature; and then cooled to 0° C. Aqueous ammonia (28%, 15 mL) was added dropwise. The resulting mixture was stirred for additional 10 min at room temperature, and then was diluted with water (30 mL). The resulting mixture was extracted with ethyl acetate (3×50 mL), dried over anhydrous sodium sulfate. After filtration, the filtrate was concentrated under reduced pressure. The residue was purified by silica gel column chromatography, eluted with 30% ethyl acetate in dichloromethane to afford the desired product as a yellow solid (300 mg, 65%). LCMS calculated for C₁₁H₁₀BrClFN₄O (M+H)⁺ m/z=347.0; found 346.9.

### Step 5: 6-Chloro-7-fluoro-2,3-dihydro-1H-3a,4,8,9a-tetraazacyclopenta[def]phenanthren-9(8H)-one

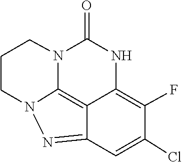 [SMILES: O=C1Nc2c(F)c(Cl)cc3nn4c(c23)N1CCC4]

To a screw-cap vial equipped with a magnetic stir bar was added 10-bromo-8-chloro-9-fluoro-3,4-dihydropyrimido[1,2-b]indazole-1(2H)-carboxamide (300 mg, 0.86 mmol), bis(dibenzylideneacetone)palladium (99 mg, 0.17 mmol), di-tert-butyl[2′,4′,6′-tris(propan-2-yl)-[1,1′-biphenyl]-2-yl]phosphane (147 mg, 0.35 mmol) and cesium carbonate (562 mg, 1.73 mmol). The vial was sealed with a Teflon-lined septum, evacuated and backfilled with nitrogen (this process was repeated a total of three times). 1,4-Dioxane (4 mL) were added. The resulting mixture was stirred at 100° C. for 2 h. Upon cooling to room temperature, the mixture was concentrated under reduced pressure. The residue was purified by silica gel column chromatography, eluted with 10% methanol in dichloromethane to afford the desired product as an off-white solid (85 mg, 37%). LCMS calculated for C₁₁H₉ClFN₄O (M+H)⁺ m/z=267.0; found 267.1.

### Step 6: 7-Fluoro-6-(hydroxymethyl)-2,3-dihydro-1H-3a,4,8,9a-tetraazacyclopenta[def]phenanthren-9(8H)-one

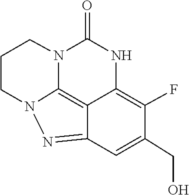 [SMILES: O=C1Nc2c(F)c(CO)cc3nn4c(c23)N1CCC4]

To a screw-cap vial equipped with a magnetic stir bar was added 6-chloro-7-fluoro-2,3-dihydro-1H-3a,4,8,9a-tetraazacyclopenta[def]phenanthren-9(8H)-one (135 mg, 0.51 mmol), (tributylstannyl)methanol (325 mg, 1.01 mmol) and XPhos Pd G3 (86 mg, 0.1 mmol). The vial was sealed with a Teflon-lined septum, evacuated and backfilled with nitrogen (this process was repeated a total of three times). 1,4-Dioxane (3 mL) were added. The resulting mixture was stirred at 100° C. for 2 h. Upon cooling to room temperature, the mixture was concentrated under reduced pressure. The residue was purified by silica gel column chromatography, eluted with 10% methanol in dichloromethane to afford the desired product as a yellow solid (40 mg, 30%). LCMS calculated for C₁₂H₁₂FN₄O₂(M+H)⁺ m/z=263.1; found 263.2.

### Step 7: 5-(4-((7-Fluoro-9-oxo-2,3,8,9-tetrahydro-1H-3a,4,8,9a-tetraazacyclopenta[def]phenanthren-6-yl)methyl)piperazin-1-yl)-N,6-dimethylpicolinamide

To 7-fluoro-6-(hydroxymethyl)-2,3-dihydro-1H-3a,4,8,9a-tetraazacyclopenta[def]phenanthren-9(8H)-one (20 mg, 0.08 mmol) was added hydrogen bromide (33 wt. % solution in glacial acid, 2 mL) at room temperature. The reaction mixture was heated at 80° C. under nitrogen atmosphere for 15 min. Upon cooling to room temperature, the reaction mixture was concentrated under reduced pressure. To the residue was added acetonitrile (3 mL), followed by N,6-dimethyl-5-(piperazin-1-yl)picolinamide (21 mg, 0.09 mmol) and N-ethyl-N-isopropylpropan-2-amine (98 mg, 0.76 mmol). The resulting mixture was stirred at room temperature for 16 h; and then was concentrated under vacuum. The residue was purified by Prep-HPLC (column: XBridge Prep Phenyl OBD Column 19*250 mm, 5 m; mobile phase A: water (0.05% trifluoroacetic acid), mobile phase B: acetonitrile; Flow rate: 60 mL/min; gradient: 2% B to 19% B over 8 min; detector: 254/220 nm). Fractions were collected and lyophilized to provide the TFA salt of the desired product as a white solid (22 mg). LCMS calculated for C₂₄H₂₈FN₈O₂(M+H)⁺ m/z=479.2; found 479.3; ¹H NMR (400 MHz, CD₃OD) δ 7.90 (d, J=8.0 Hz, 1H), 7.57 (d, J=8.0 Hz, 1H), 7.10 (d, J=3.2 Hz, 1H), 4.58 (s, 2H), 4.32 (t, J=5.6 Hz, 2H), 3.91 (t, J=5.6 Hz, 2H), 3.65-3.56 (m, 4H), 3.34-3.27 (m, 4H), 2.94 (s, 3H), 2.60 (s, 3H), 2.50-2.44 (m, 2H).

### Example 2. N-Cyclopropyl-5-(4-((7-fluoro-9-oxo-2,3,8,9-tetrahydro-1H-3a,4,8,9a-tetraazacyclopenta[def]phenanthren-6-yl)methyl)piperazin-1-yl)-6-methylpicolinamide

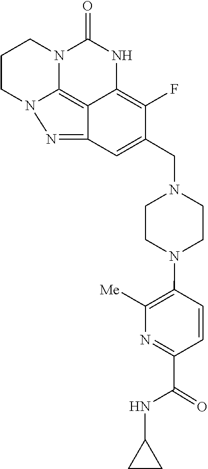 [SMILES: Cc1nc(C(=O)NC2CC2)ccc1N1CCN(Cc2cc3nn4c5c3c(c2F)NC(=O)N5CCC4)CC1]

To 7-fluoro-6-(hydroxymethyl)-2,3-dihydro-1H-3a,4,8,9a-tetraazacyclopenta[def]phenanthren-9(8H)-one (20 mg, 0.08 mmol) was added hydrogen bromide (33 wt. % solution in glacial acid, 2 mL) at room temperature. The reaction mixture was heated at 80° C. under nitrogen atmosphere for 15 min. Upon cooling to room temperature, the reaction mixture was concentrated under reduced pressure. To the residue was added acetonitrile (3 mL); followed by N-cyclopropyl-6-methyl-5-(piperazin-1-yl)picolinamide (24 mg, 0.09 mmol) and N-ethyl-N-isopropylpropan-2-amine (98 mg, 0.76 mmol). The resulting mixture was stirred at room temperature for 16 h; and then concentrated under vacuum. The residue was purified by Prep-HPLC (column: XBridge Prep Phenyl OBD Column 19*250 mm, 5 m; mobile phase A: water (0.05% trifluoroacetic acid), mobile phase B: acetonitrile; Flow rate: 60 mL/min; gradient: 6% B to 23% B over 8 min; detector: 254/220 nm). Fractions were collected and lyophilized to provide the TFA salt of the desired product as a white solid (21 mg). LCMS calculated for C₂₆H₃₀FN₈O₂(M+H)⁺ m/z=505.2; found 505.3; ¹H NMR (400 MHz, CD₃OD) δ 7.91 (d, J=8.4 Hz, 1H), 7.57 (d, J=8.4 Hz, 1H), 7.04 (d, J=3.6 Hz, 1H), 4.55 (s, 2H), 4.31 (t, J=5.6 Hz, 2H), 3.89 (t, J=5.6 Hz, 2H), 3.63-3.54 (m, 4H), 3.29-3.18 (m, 4H), 2.88-2.82 (m, 1H), 2.58 (s, 3H), 2.48-2.42 (m, 2H), 0.86-0.81 (m, 2H), 0.68-0.64 (m, 2H).

### Example 3. 5-(4-((6-Fluoro-4-oxo-2,3,4,5-tetrahydro-1-oxa-3a,5,9-triazapyren-7-yl)methyl)piperazin-1-yl)-N,6-dimethylpicolinamide

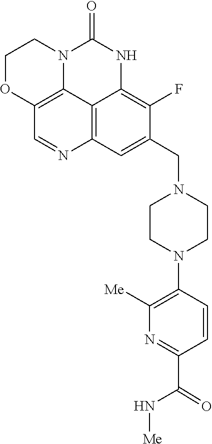 [SMILES: CNC(=O)c1ccc(N2CCN(Cc3cc4ncc5c6c4c(c3F)NC(=O)N6CCO5)CC2)c(C)n1]

### Step 1: 5,7-Dichloro-6-fluoroquinolin-4-ol

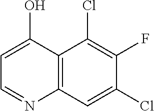 [SMILES: Oc1ccnc2cc(Cl)c(F)c(Cl)c12]

To a mixture of 3,5-dichloro-4-fluoroaniline (10 g, 55.5 mmol) in iso-propanol (100 mL) was added 5-(methoxymethylene)-2,2-dimethyl-1,3-dioxane-4,6-dione (11.38 g, 61.1 mmol) dropwise at 0° C. The resulting mixture was stirred at room temperature for 1 h. The precipitated solids were collected by filtration, washed with iso-propanol (3×100 mL) and dried under vacuum. The residue was taken in diphenyl ether (150 mL). The resulting mixture was stirred at 230° C. for 30 min. The precipitated solids were collected by filtration, washed with hexane (3×100 mL) and then dried under vacuum to afford the desired product as a brown solid (7 g, 54%). LCMS calculated for C₉H₅Cl₂FNO (M+H)⁺ m/z=232.0; found 231.9. H NMR (400 MHz, DMSO-d6) δ 11.86 (s, 1H), 7.89 (d, J=7.6 Hz, 1H), 7.68 (d, J=6.4 Hz, 1H), 6.03 (d, J=7.6 Hz, 1H).

### Step 2: 3-Bromo-5,7-dichloro-6-fluoroquinolin-4-ol

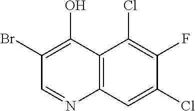 [SMILES: Oc1c(Br)cnc2cc(Cl)c(F)c(Cl)c12]

To a mixture of 5,7-dichloro-6-fluoroquinolin-4-ol (7 g, 30.17 mmol) in acetic acid (70 mL) was added 1-bromopyrrolidine-2,5-dione (5.37 g, 30.17 mmol) at room temperature. The resulting mixture was stirred at 60° C. for 2 h. Upon cooling to room temperature, the precipitated solids were collected by filtration, washed with water (5×200 mL) and then dried under vacuum to afford the desired product as a white solid (9 g, 96%). LCMS calculated for C₉H₄BrCl₂FNO (M+H)⁺ m/z=309.9; found 309.8.

### Step 3: 3-Bromo-4,5,7-trichloro-6-fluoroquinoline

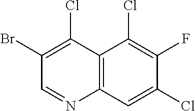 [SMILES: Fc1c(Cl)cc2ncc(Br)c(Cl)c2c1Cl]

A mixture of 3-bromo-5,7-dichloro-6-fluoroquinolin-4-ol (9 g, 28.94 mmol) in phosphoryl trichloride (100 mL) was stirred at 100° C. for 2 h. Upon cooling to room temperature, the mixture was concentrated under reduced pressure. The residue was dissolved in ethyl acetate (500 mL), washed with saturated aqueous sodium bicarbonate (2×250 mL). Organic layers were dried over anhydrous sodium sulfate and filtered. The filtrate was concentrated under reduced pressure. The residue was purified by silica gel column chromatography, eluted with 30% ethyl acetate in dichloromethane to afford the desired product as a brown solid (9 g, 94%). LCMS calculated for C₉H₃BrCl₃FN (M+H)⁺ m/z=327.8; found 327.9.

### Step 4: 2-((3-Bromo-5,7-dichloro-6-fluoroquinolin-4-yl)amino)ethan-1-ol

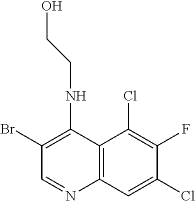 [SMILES: OCCNc1c(Br)cnc2cc(Cl)c(F)c(Cl)c12]

To a mixture of 3-bromo-4,5,7-trichloro-6-fluoroquinoline (3 g, 9.11 mmol) in dimethyl sulfoxide (30 mL) was added 2-aminoethan-1-ol (0.67 g, 10.93 mmol) at 25° C. The resulting mixture was stirred for 2 h at 100° C. Upon cooling to room temperature, the resulting mixture was diluted with water (300 mL) and extracted with ethyl acetate (3×150 mL). The combined organic layers were washed with brine (150 mL), dried over anhydrous sodium sulfate. After filtration, the filtrate was concentrated under reduced pressure. The residue was purified by silica gel column chromatography, eluted with 50% ethyl acetate in dichloromethane to afford the desired product as a white solid (3 g, 93%). LCMS calculated for C₁₁H₉BrCl₂FN₂O (M+H)⁺ m/z=352.9; found 352.9.

### Step 5: 8,10-Dichloro-9-fluoro-2,3-dihydro-1H-[1,4]oxazino[2,3-c]quinoline

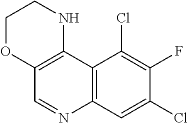 [SMILES: Fc1c(Cl)cc2ncc3c(c2c1Cl)NCCO3]

To a screw-cap vial equipped with a magnetic stir bar was added 2-((3-bromo-5,7-dichloro-6-fluoroquinolin-4-yl)amino)ethan-1-ol (1.4 g, 3.955 mmol), cesium carbonate (2.58 g, 7.91 mmol) and RockPhos Pd G3(0.33 g, 0.396 mmol). The vial was sealed with a Teflon-lined septum, evacuated and backfilled with nitrogen (this process was repeated a total of three times). Toluene (30 mL) was added. The resulting mixture was stirred at 120° C. for 16 h. Upon cooling to room temperature, the reaction mixture was concentrated under reduced pressure. The residue was purified by silica gel column chromatography, eluted with 50% ethyl acetate in dichloromethane to afford the desired product as a white solid (150 mg, 14%). LCMS calculated for C₁₁H₈Cl₂FN₂O (M+H)⁺ m/z=273.0; found 273.1.

### Step 6: 7-Chloro-6-fluoro-5-(4-methoxybenzyl)-2,3-dihydro-1-oxa-3a,5,9-triazapyren-4(5H)-one

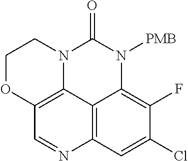

To a mixture of 8,10-dichloro-9-fluoro-2,3-dihydro-1H-[1,4]oxazino[2,3-c]quinoline (150 mg, 0.55 mmol) in N,N-dimethylformamide (3 mL) was added sodium hydride (44 mg, 1.10 mmol, 60% in mineral oil) at 0° C. under nitrogen atmosphere. The mixture was stirred for 20 min, followed by the addition of 1-(isocyanatomethyl)-4-methoxybenzene (134 mg, 0.82 mmol) at 0° C. The resulting mixture was stirred for 1 h at the same temperature; and then quenched with water (50 mL). The resulting mixture was extracted with ethyl acetate (3×50 mL). The combined organic layers were washed with brine (50 mL), dried over anhydrous sodium sulfate. After filtration, the filtrate was concentrated under reduced pressure. The residue was purified by silica gel column chromatography, eluted with 50% ethyl acetate in dichloromethane to afford the desired product as a white solid (160 mg, 73%). LCMS calculated for C₂₀H₁₆ClFN₃O₃(M+H)⁺ m/z=400.1; found 400.0. ¹H NMR (400 MHz, CDCl₃) δ 8.42 (s, 1H), 7.56 (d, J=6.0 Hz, 1H), 7.29 (d, J=8.7, 2H), 6.86 (d, J=8.7 Hz, 2H), 5.37 (s, 2H), 4.34 (t, J=4.4 Hz, 2H), 4.10 (t, J=4.4 Hz, 2H), 3.78 (s, 3H).

### Step 7: 6-Fluoro-7-(hydroxymethyl)-5-(4-methoxybenzyl)-2,3-dihydro-1-oxa-3a,5,9-triazapyren-4(5H)-one

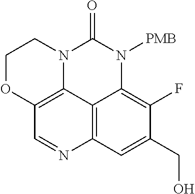

To a screw-cap vial equipped with a magnetic stir bar was added 7-chloro-6-fluoro-5-(4-methoxybenzyl)-2,3-dihydro-1-oxa-3a,5,9-triazapyren-4(5H)-one (80 mg, 0.20 mmol), (tributylstannyl)methanol (96 mg, 0.30 mmol) and XPhos Pd G3 (34 mg, 0.04 mmol). The vial was sealed with a Teflon-lined septum, evacuated and backfilled with nitrogen (this process was repeated a total of three times). 1,4-Dioxane (1 mL) were added. The resulting mixture was stirred at 100° C. for 2 h. Upon cooling to room temperature, the mixture was concentrated under reduced pressure. The residue was purified by silica gel column chromatography, eluted with 10% methanol in dichloromethane to afford the desired product as a yellow solid (70 mg, 88%). LCMS calculated for C₂₁H₁₉FN₃O₄(M+H)⁺ m/z=396.1; found 396.1. ¹H NMR (400 MHz, DMSO-d₆) δ 8.38 (s, 1H), 7.42 (d, J=5.2 Hz, 1H), 7.24 (d, J=8.7 Hz, 2H), 6.87 (d, J=8.7 Hz, 2H), 5.41 (t, J=6.0 Hz, 1H), 5.23 (s, 2H), 4.53 (d, J=6.0 Hz, 2H), 4.35 (t, J=4.4 Hz, 2H), 3.96 (t, J=4.4 Hz, 2H), 3.72 (s, 3H).

### Step 8: 5-(4-((6-Fluoro-4-oxo-2,3,4,5-tetrahydro-1-oxa-3a,5,9-triazapyren-7-yl)methyl)piperazin-1-yl)-N,6-dimethylpicolinamide

To 6-fluoro-7-(hydroxymethyl)-5-(4-methoxybenzyl)-2,3-dihydro-1-oxa-3a,5,9-triazapyren-4(5H)-one (35 mg, 0.09 mmol) was added hydrogen bromide (33 wt. % solution in glacial acid, 1 mL) at room temperature. The reaction mixture was heated at 80° C. under nitrogen atmosphere for 30 min. Upon cooling to room temperature, the reaction mixture was concentrated under reduced pressure. To the residue was added acetonitrile (2 mL), followed by N,6-dimethyl-5-(piperazin-1-yl)picolinamide (22 mg, 0.09 mmol) and N-ethyl-N-isopropylpropan-2-amine (296 mg, 2.30 mmol). The resulting mixture was stirred at room temperature for 16 h; and then was concentrated under vacuum. The residue was purified by Prep-HPLC (column: XBridge Prep Phenyl OBD Column 19*250 mm, 5 m; mobile phase A: water (0.05% trifluoroacetic acid), mobile phase B: acetonitrile; Flow rate: 40 mL/min; gradient: 2% B to 40% B over 15 min; detector: 254/220 nm). Fractions were collected and lyophilized to provide the TFA salt of the desired product as a white solid (20 mg). LCMS calculated for C₂₅H₂₇FN₇O₃(M+H)⁺ m/z=492.2; found 492.3; ¹H NMR (400 MHz, CD₃OD) δ 8.55 (s, 1H), 7.90 (d, J=8.4 Hz, 1H), 7.63 (d, J=5.2 Hz, 1H), 7.56 (d, J=8.4 Hz, 1H), 4.62 (s, 2H), 4.45 (t, J=4.4 Hz, 2H), 4.10 (t, J=4.4 Hz, 2H), 3.61-3.49 (m, 4H), 3.39-3.34 (m, 4H), 2.94 (s, 3H), 2.60 (s, 3H).

### Example 4. N-Cyclopropyl-5-(4-((6-fluoro-4-oxo-2,3,4,5-tetrahydro-1-oxa-3a,5,9-triazapyren-7-yl)methyl)piperazin-1-yl)-6-methylpicolinamide

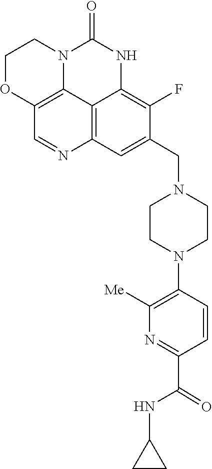 [SMILES: Cc1nc(C(=O)NC2CC2)ccc1N1CCN(Cc2cc3ncc4c5c3c(c2F)NC(=O)N5CCO4)CC1]

To 6-fluoro-7-(hydroxymethyl)-5-(4-methoxybenzyl)-2,3-dihydro-1-oxa-3a,5,9-triazapyren-4(5H)-one (35 mg, 0.09 mmol) was added hydrogen bromide (33 wt. % solution in glacial acid, 1 mL) at room temperature. The reaction mixture was heated at 80° C. under nitrogen atmosphere for 30 min. Upon cooling to room temperature, the reaction mixture was concentrated under reduced pressure. To the residue was added acetonitrile (2 mL), followed by N-cyclopropyl-6-methyl-5-(piperazin-1-yl)picolinamide (24 mg, 0.09 mmol) and N-ethyl-N-isopropylpropan-2-amine (298 mg, 2.28 mmol). The resulting mixture was stirred at room temperature for 16 h; and then was concentrated under vacuum. The residue was purified by Prep-HPLC (column: XBridge Prep Phenyl OBD Column 19*250 mm, 5 m; mobile phase A: water (0.05% trifluoroacetic acid), mobile phase B: acetonitrile; Flow rate: 40 mL/min; gradient: 6% B to 40% B over 10 min; detector: 254/220 nm). Fractions were collected and lyophilized to provide the TFA salt of the desired product as a white solid (25 mg). LCMS calculated for C₂₇H₂₉FN₇O₃(M+H)⁺ m/z=518.2; found 518.3; ¹H NMR (400 MHz, CD₃OD) δ 8.63 (s, 1H), 7.90 (d, J=8.4 Hz, 1H), 7.74-7.72 (m, 1H), 7.57 (d, J=8.4 Hz, 1H), 4.70 (s, 2H), 4.49 (t, J=4.4 Hz, 2H), 4.15 (t, J=4.4 Hz, 2H), 3.64-3.56 (m, 4H), 3.32-3.30 (m, 4H), 2.90-2.80 (m, 1H), 2.58 (s, 3H), 0.88-0.80 (m, 2H), 0.70-0.63 (m, 2H).

### Example 5. 5-(4-((7-Fluoro-5-oxo-2,3,5,6-tetrahydro-1H-[1,6]naphthyridino[1,8,7-cde]quinazolin-8-yl)methyl)piperazin-1-yl)-N,6-dimethylpicolinamide

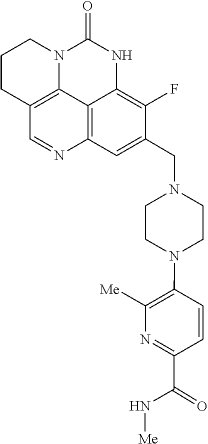 [SMILES: CNC(=O)c1ccc(N2CCN(Cc3cc4ncc5c6c4c(c3F)NC(=O)N6CCC5)CC2)c(C)n1]

### Step 1: tert-Butyl (3-(4,5,7-trichloro-6-fluoroquinolin-3-yl)propyl carbamate

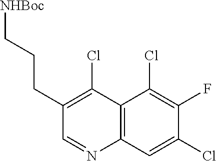 [SMILES: CC(C)(C)OC(=O)NCCCc1cnc2cc(Cl)c(F)c(Cl)c2c1Cl]

To a mixture of tert-butyl N-(prop-2-en-1-yl)carbamate (310 mg, 1.97 mmol) in tetrahydrofuran (3 mL) was added 9-borabicyclo[3.3.1]nonane (0.5 M in tetrahydrofuran, 7.89 mL, 3.947 mmol) at room temperature under nitrogen atmosphere. The resulting mixture was stirred at room temperature for 2 h. To the above mixture were added 3-bromo-4,5,7-trichloro-6-fluoroquinoline (Example 3, Step 3: 500 mg, 1.52 mmol), potassium phosphate (838 mg, 3.95 mmol), 1,1′-bis(diphenylphosphino)ferrocene-palladium(II)dichloride dichloromethane complex (124 mg, 0.15 mmol) and water (1.5 mL) at room temperature. The resulting mixture was stirred at room temperature for 16 h. After solvent removed, the residue was partitioned between ethyl acetate (50 mL) and water (50 mL). The aqueous layer was separated and extracted with ethyl acetate (2×50 mL). The combined organic extracts were washed with brine, dried over anhydrous sodium sulfate. After filtration, the filtrate was concentrated under reduced pressure. The residue was purified by silica gel column chromatography, eluted with 30% ethyl acetate in petroleum ether to afford the desired product as a white solid (200 mg, 32%). LCMS calculated for C₁₇H₁₉Cl₃FN₂O₂(M+H)⁺ m/z=407.0; found 407.1; ¹H NMR (400 MHz, CDCl₃) δ 8.70 (s, 1H), 8.12 (d, J=7.6 Hz, 1H), 4.63 (s, 1H), 3.28-3.23 (m, 2H), 3.00-2.96 (m, 2H), 1.93-1.86 (m, 2H), 1.46 (s, 9H).

### Step 2: 8,10-Dichloro-9-fluoro-1,2,3,4-tetrahydrobenzo[h][1,6]naphthyridine

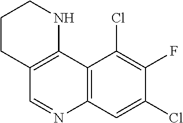 [SMILES: Fc1c(Cl)cc2ncc3c(c2c1Cl)NCCC3]

To a stirred mixture of tert-butyl (3-(4,5,7-trichloro-6-fluoroquinolin-3-yl)propyl)carbamate (180 mg, 0.42 mmol) in dichloromethane (5 mL) was added 2,2,2-trifluoroacetic acid (2 mL). The resulting mixture was stirred for 30 min. After solvent removed, the residue was taken in N,N-dimethylformamide (3 mL). To the above mixture was added potassium carbonate (293 mg, 2.12 mmol). The resulting mixture was stirred at 100° C. for 1 h. Upon cooling to room temperature, the residue was partitioned between ethyl acetate (30 mL) and water (30 mL). The aqueous layer was separated and extracted with ethyl acetate (2×50 mL). The combined organic extracts were washed with brine, dried over anhydrous sodium sulfate. After filtration, the filtrate was concentrated under reduced pressure. The residue was purified by silica gel column chromatography, eluted with 50% ethyl acetate in petroleum ether to afford the desired product as a white solid (113 mg, 98%). LCMS calculated for C₁₂H₁₀Cl₂FN₂ (M+H)⁺ m/z=271.0; found 271.0; ¹H NMR (400 MHz, CDCl₃) δ 8.21 (s, 1H), 7.88 (d, J=7.2 Hz, 1H), 7.16 (s, 1H), 3.55-3.51 (m, 2H), 2.86 (t, J=6.4 Hz, 2H), 2.05-1.96 (m, 2H).

### Step 3: 8-Chloro-7-fluoro-6-(4-methoxybenzyl)-1,2,3,6-tetrahydro-5H-[1,6]naphthyridino[1,8,7-cde]quinazolin-5-one

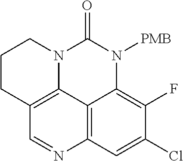

To a stirred mixture of 8,10-dichloro-9-fluoro-1,2,3,4-tetrahydrobenzo[h][1,6]naphthyridine (100 mg, 0.37 mmol) in N,N-dimethylformamide (2 mL) was added sodium hydride (60% dispersion in mineral oil, 30 mg, 0.74 mmol). The resulting mixture was stirred for 30 min at 0° C. To the above mixture was added 1-(isocyanatomethyl)-4-methoxybenzene (92 mg, 0.55 mmol) dropwise at 0° C. The resulting mixture was stirred at room temperature for 2 h. The reaction mixture was quenched with saturated aqueous ammonium chloride solution (10 mL) at 0° C. The resulting mixture was extracted with ethyl acetate (3×20 mL). The combined organic layers were washed with brine (10 mL), dried over anhydrous sodium sulfate. After filtration, the filtrate was concentrated under reduced pressure. The residue was purified by silica gel column chromatography, eluted with 50% ethyl acetate in petroleum ether to afford the desired product as a light-yellow solid (122 mg, 83%). LCMS calculated for C₂₁H₁₈ClFN₃O₂(M+H)⁺ m/z=398.1; found 398.0.

### Step 4: 7-Fluoro-8-(hydroxymethyl)-6-(4-methoxybenzyl)-1,2,3,6-tetrahydro-5H-[1,6]naphthyridino[1,8,7-cde]quinazolin-5-one

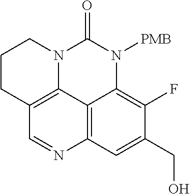

To a screw-cap vial equipped with a magnetic stir bar was added 8-chloro-7-fluoro-6-(4-methoxybenzyl)-1,2,3,6-tetrahydro-5H-[1,6]naphthyridino[1,8,7-cde]quinazolin-5-one (100 mg, 0.25 mmol), XPhos Pd G3 (43 mg, 0.05 mmol), and (tributylstannyl)methanol (161 mg, 0.5 mmol). The vial was sealed with a Teflon-lined septum, evacuated and backfilled with nitrogen (this process was repeated a total of three times). 1,4-Dioxane (2 mL) were added. The resulting mixture was stirred at 100° C. for 2 h. Upon cooling to room temperature, the mixture was concentrated under reduced pressure. The residue was purified by silica gel column chromatography, eluted with 10% methanol in dichloromethane to afford the desired product as a light-yellow solid (90 mg, 91%). LCMS calculated for C₂₂H₂₁FN₃O₃ (M+H)⁺ m/z=394.2; found 394.3.

### Step 5: 5-(4-((7-Fluoro-5-oxo-2,3,5,6-tetrahydro-1H-[1,6]naphthyridino[1,8,7-cde]quinazolin-8-yl)methyl)piperazin-1-yl)-N,6-dimethylpicolinamide

To 7-fluoro-8-(hydroxymethyl)-6-(4-methoxybenzyl)-1,2,3,6-tetrahydro-5H-[1,6]naphthyridino[1,8,7-cde]quinazolin-5-one (40 mg, 0.1 mmol) was added hydrogen bromide (33 wt. % solution in glacial acid, 1 mL) at room temperature. The reaction mixture was heated at 80° C. under nitrogen atmosphere for 15 min. Upon cooling to room temperature, the reaction mixture was concentrated under reduced pressure. To the residue was added acetonitrile (3 mL), followed by N,6-dimethyl-5-(piperazin-1-yl)picolinamide (26 mg, 0.11 mmol) and N-ethyl-N-isopropylpropan-2-amine (131 mg, 1.02 mmol). The resulting mixture was stirred at room temperature for 16 h; and then was concentrated under vacuum. The residue was purified by silica gel column chromatography, eluted with 10% methanol in dichloromethane to afford crude product which was further purified by reversed-phase flash chromatography with the following conditions: column, C18 silica gel; mobile phase, acetonitrile in water (0.1% trifluoroacetic acid), 10% to 40% gradient in 25 min; detector, UV 254 nm. Fractions were collected and lyophilized to provide the TFA salt of the desired product as a white solid (20 mg). LCMS calculated for C₂₆H₂₉FN₇O₂(M+H)⁺ m/z=490.2; found 490.2; ¹H NMR (300 MHz, CD₃OD) δ 8.61 (s, 1H), 7.93 (d, J=8.4 Hz, 1H), 7.73 (d, J=5.1 Hz, 1H), 7.59 (d, J=8.4 Hz, 1H), 4.75 (s, 2H), 4.11 (t, J=5.7 Hz, 2H), 3.63-3.60 (m, 4H), 3.31-3.27 (m, 4H), 3.00-2.98 (m, 2H), 2.96 (s, 3H), 2.62 (s, 3H), 2.19-2.15 (m, 2H).

### Example 6. N-Cyclopropyl-5-(4-((7-fluoro-5-oxo-2,3,5,6-tetrahydro-1H-[1,6]naphthyridino[1,8,7-cde]quinazolin-8-yl)methyl)piperazin-1-yl)-6-methylpicolinamide

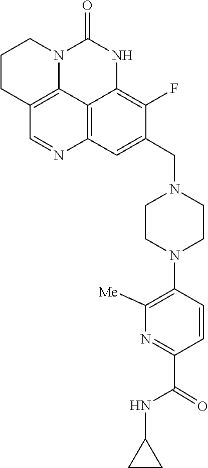 [SMILES: Cc1nc(C(=O)NC2CC2)ccc1N1CCN(Cc2cc3ncc4c5c3c(c2F)NC(=O)N5CCC4)CC1]

To 7-fluoro-8-(hydroxymethyl)-6-(4-methoxybenzyl)-1,2,3,6-tetrahydro-5H-[1,6]naphthyridino[1,8,7-cde]quinazolin-5-one (40 mg, 0.1 mmol) was added hydrogen bromide (33 wt. % solution in glacial acid, 1 mL) at room temperature. The reaction mixture was heated at 80° C. under nitrogen atmosphere for 15 min. Upon cooling to room temperature, the reaction mixture was concentrated under reduced pressure. To the residue was added acetonitrile (3 mL), followed by N-cyclopropyl-6-methyl-5-(piperazin-1-yl)picolinamide (29 mg, 0.11 mmol) and N-ethyl-N-isopropylpropan-2-amine (131 mg, 1.02 mmol). The resulting mixture was stirred at room temperature for 16 h; and then was concentrated under vacuum. The residue was purified by silica gel column chromatography, eluted with 10% methanol in dichloromethane to afford crude product which was further purified by reversed-phase flash chromatography with the following conditions: column, C18 silica gel; mobile phase, acetonitrile in water (0.1% trifluoroacetic acid), 10% to 40% gradient in 25 min; detector, UV 254 nm. Fractions were collected and lyophilized to provide the TFA salt of the desired product as a white solid. LCMS calculated for C₂₈H₃₁FN₇O₂ (M+H)⁺ m/z=516.3; found 516.4; ¹H NMR (300 MHz, CD₃OD) δ 8.61 (s, 1H), 7.93 (d, J=8.4 Hz, 1H), 7.71 (d, J=5.1 Hz, 1H), 7.59 (d, J=8.4 Hz, 1H), 4.70 (s, 2H), 4.11 (t, J=5.7 Hz, 2H), 3.61-3.56 (m, 4H), 3.32-3.28 (m, 4H), 2.97 (t, J=6.0 Hz, 2H), 2.90-2.84 (m, 1H), 2.61 (s, 3H), 2.19-2.13 (m, 2H), 0.89-0.82 (m, 2H), 0.71-0.65 (m, 2H).

### Example 7. 5-(4-((3-Ethyl-9-fluoro-2-oxo-2,3-dihydro-1H-pyrimido[4,5,6-de]quinazolin-8-yl)methyl)piperazin-1-yl)-N-(2-hydroxyethyl)-6-methylpicolinamide

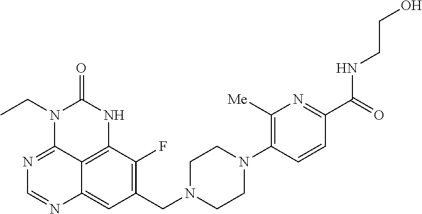 [SMILES: CCN1C(=O)Nc2c(F)c(CN3CCN(c4ccc(C(=O)NCCO)nc4C)CC3)cc3ncnc1c23]

### Step 1: 2-Amino-4-bromo-6-fluorobenzonitrile

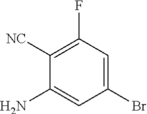 [SMILES: N#Cc1c(N)cc(Br)cc1F]

A mixture of 4-bromo-2,6-difluorobenzonitrile (10 g, 45.87 mmol) and ammonium hydroxide (16.08 g, 458.7 mmol) in propan-2-ol (25 mL) was stirred at 80° C. for 16 h. Upon cooling to room temperature, the reaction mixture was diluted with water (500 mL). The precipitated solids were collected by filtration and washed with water (2×100 mL). The residue was purified by silica gel column chromatography, eluted with 20% ethyl acetate in petroleum ether to provide the desired product as a white solid (5.8 g, 59%). LCMS calculated for C₇H₃BrFN₂ (M−H)⁻ m/z=212.9; found 212.9.

### Step 2: 7-Bromo-5-fluoroquinazolin-4-amine

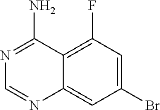 [SMILES: Nc1ncnc2cc(Br)cc(F)c12]

A mixture of 2-amino-4-bromo-6-fluorobenzonitrile (3.9 g, 18.14 mmol), ammonium acetate (21 g, 272.1 mmol) and triethyl orthoformate (40.32 g, 272.1 mmol) in ethanol (40 mL) was stirred at 110° C. for 16 h. Upon cooling to room temperature, the reaction mixture was concentrated under reduced pressure. The residue was purified by silica gel column chromatography, eluted with 50% ethyl acetate in petroleum ether to provide the desired product as a yellow solid (3.3 g, 75%). LCMS calculated for C₈H₆BrFN₃ (M+H)⁺ m/z=242.0; found 242.0.

### Step 3: 7-Bromo-NV-(4-methoxybenzyl)quinazoline-4,5-diamine

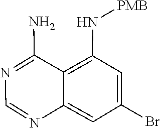

A mixture of 7-bromo-5-fluoroquinazolin-4-amine (1 g, 4.13 mmol) and (4-methoxyphenyl)methanamine (763.2 mg, 6.2 mmol) in dimethyl sulfoxide (10 mL) was stirred for 5 h at 80° C. Upon cooling to room temperature, the reaction mixture was diluted with water (100 mL), extracted with ethyl acetate (3×100 mL) and dried over anhydrous sodium sulfate. After filtration, the filtrate was concentrated under reduced pressure. The residue was purified by silica gel column chromatography, eluted with 80% ethyl acetate in dichloromethane to afford a yellow solid (1 g, 67%). LCMS calculated for C₁₆H₁₆BrN₄O (M+H)⁺ m/z=359.1; found 359.0.

### Step 4: 7-Bromo-6-fluoro-NV-(4-methoxybenzyl)quinazoline-4,5-diamine

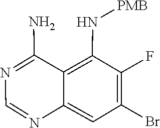

A mixture of 7-bromo-N⁵-(4-methoxybenzyl)quinazoline-4,5-diamine (1 g, 2.78 mmol) in PS-750-M (3% in water, 12 mL) was stirred at room temperature for 2 min under nitrogen atmosphere, followed by the addition of N-fluorobenzenesulfonimide (2.19 g, 6.96 mmol) in THF (12 mL) in portions over 5 min at room temperature. The resulting mixture was stirred at 60° C. for 16 h. Upon cooling to room temperature, the reaction mixture was concentrated under reduced pressure. The residue was purified by silica gel column chromatography, eluted with 7% methanol in dichloromethane to afford the desired product (450 mg, 43%) as a yellow solid. LCMS calculated for C₁₆H₁₅BrFN₄O (M+H)⁺ m/z=377.0; found 377.0; ¹H NMR (300 MHz, CDCl₃) δ 8.49 (s, 1H), 7.91 (d, J=6.6 Hz, 1H), 7.59 (s, 2H), 7.26-7.19 (m, 2H), 6.96-6.88 (m, 2H), 4.10 (d, J=6.3 Hz, 2H), 3.85 (s, 3H).

### Step 5: 8-Bromo-9-fluoro-1-(4-methoxybenzyl)-1H-pyrimido[4,5,6-de]quinazolin-2(3H)-one

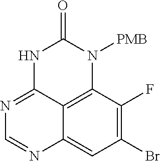

A mixture of 7-bromo-6-fluoro-N⁵-[(4-methoxyphenyl)methyl]quinazoline-4,5-diamine (710 mg, 1.88 mmol), triphosgene (1117 mg, 3.76 mmol) and N,N-diisopropylethylamine (486.5 mg, 3.76 mmol) in tetrahydrofuran (10 mL) was stirred for 1 h at room temperature under nitrogen atmosphere; and then quenched with sat. ammonium chloride (aq.) at 0° C. The precipitated solids were collected by filtration and washed with water (3×100 mL). The residue was purified by trituration with ethyl acetate (100 mL). The resulting product was a yellow solid (400 mg, 53%) which was used in the next step without further purification. LCMS calculated for C₁₇H₁₃BrFN₄O₂(M+H)⁺ m/z=403.0; found 403.1.

### Step 6: 8-Bromo-3-ethyl-9-fluoro-1-(4-methoxybenzyl)-1H-pyrimido[4,5,6-de]quinazolin-2(3H)-one

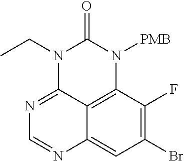

A mixture of 8-bromo-9-fluoro-1-(4-methoxybenzyl)-1H-pyrimido[4,5,6-de]quinazolin-2(3H)-one (170 mg, 0.42 mmol) and potassium carbonate (116.5 mg, 0.84 mmol) in N,N-dimethylformamide (3 mL) was treated with ethyl iodide (131.5 mg, 0.84 mmol) at 0° C. The resulting mixture was stirred at room temperature for 16 h, and then diluted with water (30 mL), extracted with ethyl acetate (3×50 mL). The combined organic layers were washed with brine (2×50 mL), dried over anhydrous sodium sulfate. After filtration, the filtrate was concentrated under reduced pressure. The resulting residue was purified by silica gel column chromatography, eluted with 10% methanol in dichloromethane to afford a yellow solid (145 mg, 80%). LCMS calculated for C₁₉H₁₇BrFN₄O₂ (M+H)⁺ m/z=431.1; found 431.0.

### Step 7: 3-Ethyl-9-fluoro-1-(4-methoxybenzyl)-8-vinyl-1H-pyrimido[4,5,6-de]quinazolin-2(3H)-one

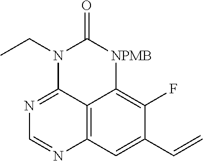

To a 500-mL round bottom flask equipped with a magnetic stirring bar was added 8-bromo-3-ethyl-9-fluoro-1-(4-methoxybenzyl)-1H-pyrimido[4,5,6-de]quinazolin-2(3H)-one (5.85 g, 13.56 mmol), Pd(dppf)Cl₂ CH₂Cl₂ (1.11 g, 1.36 mmol), vinylboronic acid pinacol ester (6.89 g, 27.13 mmol) and cesium carbonate (8.84 g, 27.13 mmol). The flask was sealed with a rubber septum, evacuated and backfilled with nitrogen (this process was repeated a total of three times). 1,4-Dioxane (100 mL) was added followed by water (20 mL). The mixture was stirred at 80° C. for 4 h. After cooling to room temperature, the reaction mixture was diluted with water (295 mL). The resulting precipitate was collected by filtration and dried under vacuum. The crude solid was purified by silica gel column chromatography (0-100% EtOAc in hexanes) to provide the desired product as a slight yellow solid (3.00 g, 58%). LCMS calculated for C₂₁H₂₀FN₄O₂(M+H)⁺ m/z=379.2; found 379.2.

### Step 8: 3-Ethyl-9-fluoro-1-(4-methoxybenzyl)-2-oxo-2,3-dihydro-1H-pyrimido[4,5,6-de]quinazoline-8-carbaldehyde

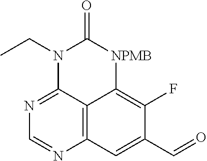

To 3-ethyl-9-fluoro-1-(4-methoxybenzyl)-8-vinyl-1H-pyrimido[4,5,6-de]quinazolin-2(3H)-one (4.70 g, 12.42 mmol) was added THE (90 mL) and water (30 mL) at room temperature. Then sodium periodate (13.28 g, 62.1 mmol) was added followed by osmium tetraoxide (4% in water, 3.25 mL, 0.50 mmol) and 2,6-dimethylpyridine (2.86 mL, 24.84 mmol). The reaction mixture was stirred at 45° C. for 18 hours. After cooling to room temperature, the reaction mixture was diluted with water (200 mL). The resulting precipitate was collected by filtration and washed with water (250 mL). To the precipitate was added water (800 mL). The mixture was stirred at room temperature for 1 hour. The solid was collected by filtration and dry under to provide the desired product as a slight yellow solid (3.92 g, 83%). LCMS calculated for C₂₀H₁₈FN₄O₃(M+H)⁺ m/z=381.1; found 381.1.

### Step 9: 3-Ethyl-9-fluoro-2-oxo-2,3-dihydro-1H-pyrimido[4,5,6-de]quinazoline-8-carbaldehyde

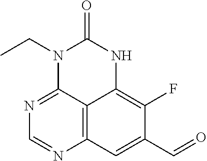 [SMILES: CCN1C(=O)Nc2c(F)c(C=O)cc3ncnc1c23]

To 3-ethyl-9-fluoro-1-(4-methoxybenzyl)-2-oxo-2,3-dihydro-1H-pyrimido[4,5,6-de]quinazoline-8-carbaldehyde (3.92 g, 10.31 mmol) was added trifluoroacetic acid (100.0 mL) at room temperature. The resulting mixture was stirred at 75° C. for 18 hours before cooling to room temperature. To the reaction mixture was added dichloromethane (50 mL) and water (300 mL). The separated aqueous phase was neutralized with saturated aqueous sodium bicarbonate solution until pH between 7 and 8. The resulting mixture was extracted with 10% methanol in dichloromethane (8×200 mL). The combined organic layers were concentrated to provide the desired product as a slight yellow solid (1.98 g, 72%). LCMS calculated for C₁₂H₁₀FN₄O₂(M+H)⁺ m/z=261.1; found 261.1.

### Step 10: Methyl 5-(4-((3-ethyl-9-fluoro-2-oxo-2,3-dihydro-1H-pyrimido[4,5,6-de]quinazolin-8-yl)methyl)piperazin-1-yl)-6-methylpicolinate

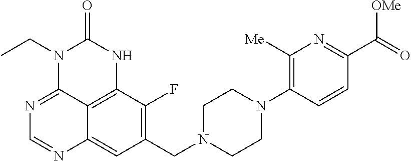 [SMILES: CCN1C(=O)Nc2c(F)c(CN3CCN(c4ccc(C(=O)OC)nc4C)CC3)cc3ncnc1c23]

To a stirred mixture of 3-ethyl-9-fluoro-2-oxo-2,3-dihydro-1H-pyrimido[4,5,6-de]quinazoline-8-carbaldehyde (817 mg, 3.14 mmol) and methyl 6-methyl-5-(piperazin-1-yl)picolinate (Intermediate 3: 923 mg, 3.92 mmol) in dichloromethane (30 mL) was added methanol (5 mL) followed by acetic acid (19 mg, 0.31 mmol). The mixture was stirred for 1 h at room temperature. Then sodium triacetoxyborohydride (2.66 g, 12.56 mmol) was added. The resulting mixture was stirred for 16 h, and then was concentrated. The residue was purified by silica gel column chromatography, eluted with 10% MeOH in CH₂Cl₂ to give the desired product as a white solid (950 mg, 63%). LCMS calculated for C₂₄H₂₇FN₇O₃(M+H)⁺ m/z=480.2; found 480.1.

### Step 11: 5-(4-((3-Ethyl-9-fluoro-2-oxo-2,3-dihydro-1H-pyrimido[4,5,6-de]quinazolin-8-yl)methyl)piperazin-1-yl)-6-methylpicolinic acid

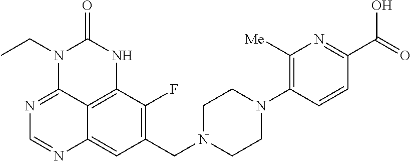 [SMILES: CCN1C(=O)Nc2c(F)c(CN3CCN(c4ccc(C(=O)O)nc4C)CC3)cc3ncnc1c23]

To a mixture of methyl 5-(4-((3-ethyl-9-fluoro-2-oxo-2,3-dihydro-1H-pyrimido[4,5,6-de]quinazolin-8-yl)methyl)piperazin-1-yl)-6-methylpicolinate (850 mg, 1.77 mmol) in methanol (5 mL) was added tetrahydrofuran (2 mL) and water (3 mL). Then lithium hydroxide monohydrate (149 mg, 3.55 mmol) was added. The reaction was stirred at 70° C. for 2 h. After cooling to room temperature, the mixture was concentrated. The residue was neutralized with hydrogen chloride (1 M, 3.6 mL) at 0° C. The resulting mixture was concentrated to give the crude desired product as a yellow solid (0.95 g) which was used directly without further purification. LCMS calculated for C₂₃H₂₅FN₇O₃(M+H)⁺ m/z=466.2; found 466.2.

### Step 12: 5-(4-((3-Ethyl-9-fluoro-2-oxo-2,3-dihydro-1H-pyrimido[4,5,6-de]quinazolin-8-yl)methyl)piperazin-1-yl)-N-(2-hydroxyethyl)-6-methylpicolinamide

To the mixture of 5-(4-((3-ethyl-9-fluoro-2-oxo-2,3-dihydro-1H-pyrimido[4,5,6-de]quinazolin-8-yl)methyl)piperazin-1-yl)-6-methylpicolinic acid (20 mg, 0.04 mmol) in N,N-dimethylformamide (1 mL) was added 2-(7-azabenzotriazol-1-yl)-N,N,N′,N′-tetramethyluronium hexafluorophosphate (25 mg, 0.06 mmol) at room temperature. The reaction was stirred for 15 min, followed by the addition of 2-aminoethan-1-ol (5 mg, 0.09 mmol) and N,N-diisopropylethylamine (16.7 mg, 0.13 mmol). The reaction mixture was stirred for an additional 2 h. The mixture was then purified by Prep-HPLC with the following conditions (Column: Xselect CSH F-Phenyl OBD column 30*250 mm, 5 m; Mobile Phase: acetonitrile in water (0.05% trifluoroacetic acid); Flow rate: 60 mL/min; Gradient: 10% B to 28% B in 10 min; Detector: 254/220 nm). Eluted fractions were collected and lyophilized to afford the TFA salt of the desired product as a white solid. LCMS calculated for C₂₅H₃₀FN₈O₃ (M+H)⁺ m/z=509.2; found 509.3; ¹H NMR (400 MHz, DMSO-d₆) δ 11.83 (s, 1H), 8.76 (s, 1H), 8.42 (t, J=6.0 Hz, 1H), 7.85 (d, J=8.4 Hz, 1H), 7.63-7.53 (m, 2H), 4.64 (s, 2H), 4.16 (q, J=6.8 Hz, 2H), 3.59-3.23 (m, 10H), 3.12-3.03 (m, 2H), 2.53 (s, 3H), 1.23 (t, J=6.8 Hz, 3H).

### Example 8. N-(2-cyanoethyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide

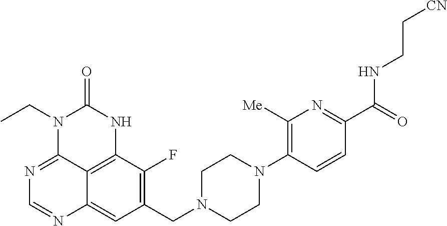 [SMILES: CCN1C(=O)Nc2c(F)c(CN3CCN(c4ccc(C(=O)NCCC#N)nc4C)CC3)cc3ncnc1c23]

The title compound was prepare according to the procedure described in Example 7, using 3-aminopropanenitrile instead of 2-aminoethan-1-ol in Step 12. LCMS calculated for C₂₆H₂₉FN₉O₂ (M+H)⁺ m/z=518.2; found 518.3. ¹H NMR (400 MHz, DMSO-d₆) δ 11.82 (s, 1H), 8.82 (t, J=6.0 Hz, 1H), 8.75 (s, 1H), 7.86 (d, J=8.4 Hz, 1H), 7.62-7.54 (m, 2H), 4.62 (s, 2H), 4.15 (q, J=6.8 Hz, 2H), 3.58-3.27 (m, 8H), 3.15-3.06 (m, 2H), 2.79 (t, J=6.4 Hz, 2H), 2.55 (s, 3H), 1.23 (t, J=6.8 Hz, 3H).

### Example 9. N-(cyanomethyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide

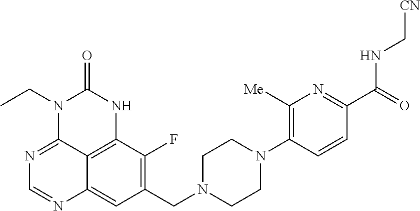 [SMILES: CCN1C(=O)Nc2c(F)c(CN3CCN(c4ccc(C(=O)NCC#N)nc4C)CC3)cc3ncnc1c23]

The title compound was prepared according to the procedure described in Example 7, using 2-aminoacetonitrile instead of 2-aminoethan-1-ol in Step 12. LCMS calculated for C₂₅H₂₇FN₉O₂(M+H)⁺ m/z=504.2; found 504.3. ¹H NMR (400 MHz, DMSO-d₆) δ 11.84 (s, 1H), 9.15 (t, J=6.0 Hz, 1H), 8.76 (s, 1H), 7.88 (d, J=8.4 Hz, 1H), 7.63-7.55 (m, 2H), 4.66 (s, 2H), 4.29 (d, J=6.0 Hz, 2H), 4.16 (q, J=6.8 Hz, 2H), 3.61-3.03 (m, 8H), 2.54 (s, 3H), 1.23 (t, J=6.8 Hz, 3H).

### Example 10. 5-(4-((3-Ethyl-9-fluoro-2-oxo-2,3-dihydro-1H-pyrimido[4,5,6-de]quinazolin-8-yl)methyl)piperazin-1-yl)-N-((1r,3r)-3-hydroxycyclobutyl)-6-methylpicolinamide

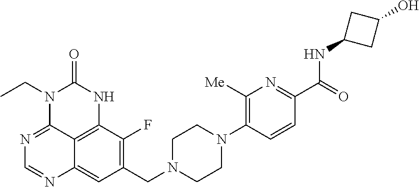 [SMILES: CCN1C(=O)Nc2c(F)c(CN3CCN(c4ccc(C(=O)N[C@H]5C[C@H](O)C5)nc4C)CC3)cc3ncnc1c23]

The title compound was prepared according to the procedure described in Example 7, using (1r,3r)-3-aminocyclobutan-1-ol instead of 2-aminoethan-1-ol in Step 12. LCMS calculated for C₂₇H₃₂FN₈O₃(M+H)⁺ m/z=535.3; found 535.3. ¹H NMR (400 MHz, DMSO-d₆) δ 11.82 (s, 1H), 8.75 (s, 1H), 8.53 (d, J=7.6 Hz, 1H), 7.81 (d, J=8.4 Hz, 1H), 7.62-7.52 (m, 2H), 4.63 (s, 2H), 4.56-4.42 (m, 1H), 4.33-4.26 (m, 1H), 4.16 (q, J=6.8 Hz, 2H), 3.57-2.98 (m, 8H), 2.54 (s, 3H), 2.37-2.27 (m, 2H), 2.20-2.10 (m, 2H), 1.23 (t, J=6.8 Hz, 3H).

### Example 13. 5-(4-((3-Ethyl-2-oxo-2,3-dihydro-1H-pyrimido[4,5,6-de]quinazolin-8-yl)methyl)piperazin-1-yl)-N-(2-hydroxyethyl)-6-methylpicolinamide

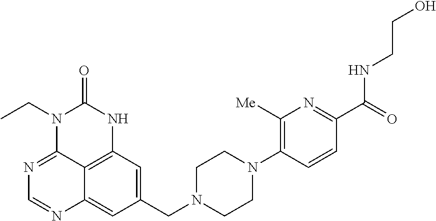 [SMILES: CCN1C(=O)Nc2cc(CN3CCN(c4ccc(C(=O)NCCO)nc4C)CC3)cc3ncnc1c23]

### Step 1: 8-Bromo-1-(4-methoxybenzyl)-1H-pyrimido[4,5,6-de]quinazolin-2(3H)-one

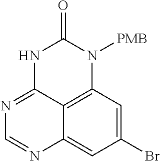

To the mixture of 7-bromo-N5-[(4-methoxyphenyl)methyl]quinazoline-4,5-diamine (Example 7, Step 3: 18 g, 50.1 mmol) and N,N-diisopropylethylamine (19.4 g, 150.3 mmol) in THE (300 mL) were added triphosgene (15.6 g, 52.6 mmol) in tetrahydrofuran (100 mL) dropwise at 0° C. The reaction was allowed to warm to room temperature and stirred for 3 h. The resulting mixture was treated with saturated aqueous sodium bicarbonate (500 mL). The precipitated solids were collected by filtration and washed with water (3×100 mL). The solid was dried under reduced pressure to afford the desired product as a yellow solid (12 g, 62%). LCMS calculated for C₁₇H₁₄BrN₄O₂(M+H)⁺ m/z=385.0; found 385.0.

### Step 2: 8-Bromo-3-ethyl-1-(4-methoxybenzyl)-1H-pyrimido[4,5,6-de]quinazolin-2(3H)-one

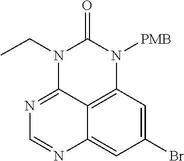

To the mixture of 8-bromo-1-(4-methoxybenzyl)-1H-pyrimido[4,5,6-de]quinazolin-2(3H)-one (2 g, 5.2 mmol) and potassium carbonate (1.44 g, 10.4 mmol) in N,N-dimethylformamide (20 mL) was added iodoethane (1.6 g, 10.4 mmol) dropwise at 0° C. The reaction was allowed to warm to room temperature and stirred for 16 h. The resulting mixture was diluted with water (100 mL). The aqueous solution was extracted with ethyl acetate (3×100 mL). The combined organic layers were washed with brine (2×200 mL), dried over anhydrous sodium sulfate. After filtration, the filtrate was concentrated under reduced pressure. The residue was purified by silica gel column chromatography, eluted with 75% ethyl acetate in petroleum ether to afford the desired product as a light-yellow solid (1.5 g, 70%). LCMS calculated for C₁₉H₁₈BrN₄O₂(M+H)⁺ m/z=413.1; found 413.1.

### Step 3: 3-Ethyl-8-(hydroxymethyl)-1-(4-methoxybenzyl)-1H-pyrimido[4,5,6-de]quinazolin-2(3H)-one

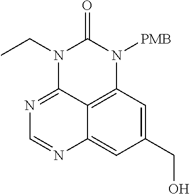

To a screw-cap vial equipped with a magnetic stir bar were placed 8-bromo-3-ethyl-1-(4-methoxybenzyl)-1H-pyrimido[4,5,6-de]quinazolin-2(3H)-one (1.5 g, 3.6 mmol) and XPhos Pd G3 (307 mg, 0.36 mmol). The vial was sealed with a Teflon-lined septum, evacuated and backfilled with nitrogen (this process was repeated a total of three times). A solution of (tributylstannyl)methanol (1.748 g, 5.45 mmol) in 1,4-dioxane (20 mL) was added via syringe. The reaction was stirred at 80° C. for 3 h. Upon cooling to room temperature, the mixture was concentrated under reduced pressure. The residue was purified by silica gel column chromatography, eluted with 10% methanol in dichloromethane to afford the desired product as a light-yellow solid (1.2 g, 91%). LCMS calculated for C₂₀H₂₁N₄O₃ (M+H)⁺ m/z=365.2; found 365.1.

### Step 4: 8-(Bromomethyl)-3-ethyl-1H-pyrimido[4,5,6-de]quinazolin-2(3H)-one

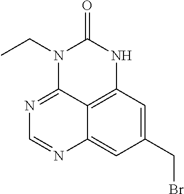 [SMILES: CCN1C(=O)Nc2cc(CBr)cc3ncnc1c23]

The mixture of 3-ethyl-8-(hydroxymethyl)-1-(4-methoxybenzyl)-1H-pyrimido[4,5,6-de]quinazolin-2(3H)-one (500 mg, 1.4 mmol) and hydrogen bromide (33 wt. % solution in glacial acid, 4 mL) was stirred at 80° C. for 8 h. Upon cooling to room temperature, the mixture was concentrated under vacuum to give the crude product which was used in the next step directly without further purification. LCMS calculated for C₁₂H₁₂BrN₄O (M+H)⁺ m/z=307.0; found 307.0.

### Step 5: Methyl 5-(4-((3-ethyl-2-oxo-2,3-dihydro-1H-pyrimido[4,5,6-de]quinazolin-8-yl)methyl)piperazin-1-yl)-6-methylpicolinate

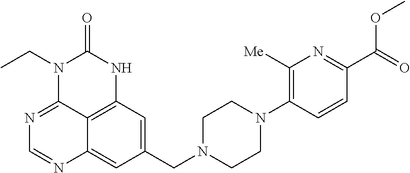 [SMILES: CCN1C(=O)Nc2cc(CN3CCN(c4ccc(C(=O)OC)nc4C)CC3)cc3ncnc1c23]

To the mixture of 8-(bromomethyl)-3-ethyl-1H-pyrimido[4,5,6-de]quinazolin-2(3H)-one (400 mg, 1.3 mmol) and methyl 6-methyl-5-(piperazin-1-yl)pyridine-2-carboxylate (Intermediate 3: 337 mg, 1.43 mmol) in acetonitrile (8 mL) was added N,N-diisopropylethylamine (1.68 g, 13 mmol). The reaction mixture was stirred at 50° C. for 16 h. Upon cooling to room temperature, the mixture was concentrated under reduced pressure. The residue was purified by silica gel column chromatography, eluted with 10% methanol in dichloromethane to afford the desired product as an off-white solid (500 mg, 83%). LCMS calculated for C₂₄H₂₈N₇O₃ (M+H)⁺ m/z=462.2; found 462.2.

### Step 6: 5-(4-((3-Ethyl-2-oxo-2,3-dihydro-1H-pyrimido[4,5,6-de]quinazolin-8-yl)methyl)piperazin-1-yl)-6-methylpicolinic acid

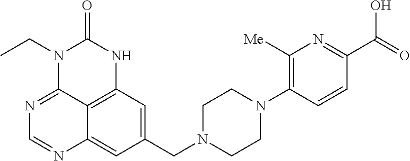 [SMILES: CCN1C(=O)Nc2cc(CN3CCN(c4ccc(C(=O)O)nc4C)CC3)cc3ncnc1c23]

To the mixture of methyl 5-(4-((3-ethyl-2-oxo-2,3-dihydro-1H-pyrimido[4,5,6-de]quinazolin-8-yl)methyl)piperazin-1-yl)-6-methylpicolinate (580 mg, 1.26 mmol) in tetrahydrofuran (6 mL), methanol (2 mL) and water (2 mL) was added lithium hydroxide monohydrate (106 mg, 2.5 mmol) at room temperature. The reaction mixture was stirred at 60° C. for 2 h. Upon cooling to room temperature, the mixture was neutralized to pH 6 with hydrochloric acid aqueous solution (1 N). The resulting mixture was concentrated under reduced pressure to give the crude product which was used in the next step directly without further purification. LCMS calculated for C₂₃H₂₆N₇O₃ (M+H)⁺ m/z=448.2; found 448.2.

### Step 7: 5-(4-((3-Ethyl-2-oxo-2,3-dihydro-1H-pyrimido[4,5,6-de]quinazolin-8-yl)methyl)piperazin-1-yl)-N-(2-hydroxyethyl)-6-methylpicolinamide

The mixture of 5-(4-((3-ethyl-2-oxo-2,3-dihydro-1H-pyrimido[4,5,6-de]quinazolin-8-yl)methyl)piperazin-1-yl)-6-methylpicolinic acid (50 mg, 0.11 mmol) and 2-(7-azabenzotriazol-1-yl)-N,N,N′,N′-tetramethyluronium hexafluorophosphate (51 mg, 0.13 mmol) in N,N-dimethylformamide (2 mL) was stirred at room temperature for 15 min. To the above mixture was added 2-aminoethan-1-ol (10 mg, 0.17 mmol) and N,N-diisopropylethylamine (72 mg, 0.56 mmol). The resulting mixture was stirred at room temperature for 2 h then purified by Prep-HPLC with the following conditions (Column: Xselect CSH C18 OBD Column 30*150 mm 5 m; Mobile Phase: acetonitrile in water (0.1% trifluoroacetic acid); Flow rate: 60 mL/min; Gradient: 40% B to 60% B in 8 min; Detector: 254/220 nm). Eluted fractions were collected and lyophilized to afford the TFA salt of the desired product as a white solid. LCMS calculated for C₂₅H₃₁N₈O₃ (M+H)⁺ m/z=491.2; found 491.3. ¹H NMR (400 MHz, Methanol-d₄) δ 8.63 (s, 1H), 7.87 (d, J=8.4 Hz, 1H), 7.49 (d, J=8.4 Hz, 1H), 7.34 (d, J=1.2 Hz, 1H), 7.03 (d, J=1.2 Hz, 1H), 4.26 (q, J=7.2 Hz, 2H), 3.75 (s, 2H), 3.70 (t, J=5.6 Hz, 2H), 3.52 (t, J=5.6 Hz, 2H), 3.09-3.02 (m, 4H), 2.74-2.68 (m, 4H), 2.54 (s, 3H), 1.31 (t, J=7.2 Hz, 3H).

### Example 14. N-(2-cyanoethyl)-5-(4-((3-ethyl-2-oxo-2,3-dihydro-1H-pyrimido[4,5,6-de]quinazolin-8-yl)methyl)piperazin-1-yl)-6-methylpicolinamide

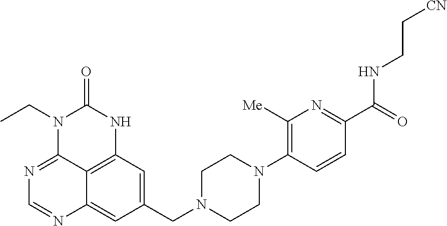 [SMILES: CCN1C(=O)Nc2cc(CN3CCN(c4ccc(C(=O)NCCC#N)nc4C)CC3)cc3ncnc1c23]

The title compound was prepared according to the procedure described in Example 13, using 3-aminopropanenitrile instead of 2-aminoethan-1-ol in Step 7. LCMS calculated for C₂₆H₃₀N₉O₂ (M+H)⁺ m/z=500.3; found 500.3. ¹H NMR (400 MHz, Methanol-d₄) δ 8.80 (s, 1H), 7.93 (d, J=8.4 Hz, 1H), 7.57 (d, J=8.4 Hz, 1H), 7.51 (d, J=1.2 Hz, 1H), 7.10 (d, J=1.2 Hz, 1H), 4.58 (s, 2H), 4.31 (q, J=7.2 Hz, 2H), 3.67 (t, J=6.4 Hz, 2H), 3.58-3.49 (m, 4H), 3.31-3.23 (m, 4H), 2.78 (t, J=6.4 Hz, 2H), 2.61 (s, 3H), 1.33 (t, J=7.2 Hz, 3H).

### Example 15. N-(cyanomethyl)-5-(4-((3-ethyl-2-oxo-2,3-dihydro-1H-pyrimido[4,5,6-de]quinazolin-8-yl)methyl)piperazin-1-yl)-6-methylpicolinamide

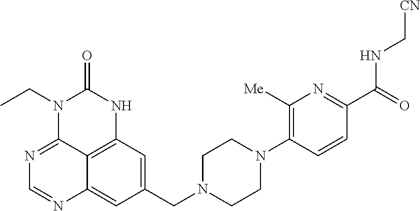 [SMILES: CCN1C(=O)Nc2cc(CN3CCN(c4ccc(C(=O)NCC#N)nc4C)CC3)cc3ncnc1c23]

The title compound was prepared according to the procedure described in Example 13, using 2-aminoacetonitrile instead of 2-aminoethan-1-ol in Step 7. LCMS calculated for C₂₅H₂₈N₉O₂ (M+H)⁺ m/z=486.2; found 486.3. ¹H NMR (400 MHz, Methanol-d₄) δ 8.82 (s, 1H), 7.94 (d, J=8.4 Hz, 1H), 7.57 (d, J=8.4 Hz, 1H), 7.51 (d, J=1.2 Hz, 1H), 7.13 (d, J=1.2 Hz, 1H), 4.58 (s, 2H), 4.39-4.26 (m, 4H), 3.62-3.49 (m, 4H), 3.31-3.21 (m, 4H), 2.61 (s, 3H), 1.33 (t, J=7.2 Hz, 3H).

### Example 16. N-cyclopropyl-5-(4-((3-ethyl-2-oxo-2,3-dihydro-1H-pyrimido[4,5,6-de]quinazolin-8-yl)methyl)piperazin-1-yl)-6-methylpicolinamide

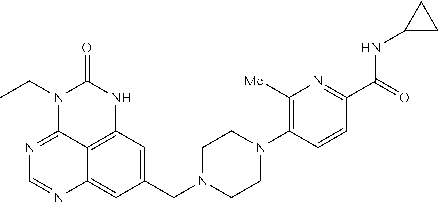 [SMILES: CCN1C(=O)Nc2cc(CN3CCN(c4ccc(C(=O)NC5CC5)nc4C)CC3)cc3ncnc1c23]

The title compound was prepared according to the procedure described in Example 13, using cyclopropanamine instead of 2-aminoethan-1-ol in Step 7. LCMS calculated for C₂₆H₃₁N₈O₂ (M+H)⁺ m/z=487.3; found 487.3. ¹H NMR (400 MHz, Methanol-d₄) δ 8.82 (s, 1H), 7.91 (d, J=8.4 Hz, 1H), 7.56 (d, J=8.4 Hz, 1H), 7.51 (d, J=1.2 Hz, 1H), 7.13 (d, J=1.2 Hz, 1H), 4.58 (s, 2H), 4.32 (q, J=7.2 Hz, 2H), 3.58-3.49 (m, 4H), 3.32-3.20 (m, 4H), 2.89-2.80 (m, 1H), 2.58 (s, 3H), 1.33 (t, J=7.2 Hz, 3H), 0.86-0.80 (m, 2H), 0.69-0.62 (m, 2H).

### Example 17. N-(1-cyanocyclopropyl)-5-(4-((3-ethyl-2-oxo-2,3-dihydro-1H-pyrimido[4,5,6-de]quinazolin-8-yl)methyl)piperazin-1-yl)-6-methylpicolinamide

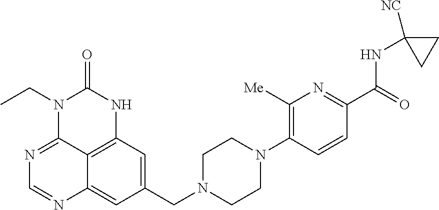 [SMILES: CCN1C(=O)Nc2cc(CN3CCN(c4ccc(C(=O)NC5(C#N)CC5)nc4C)CC3)cc3ncnc1c23]

The title compound was prepared according to the procedure described in Example 13, using 1-aminocyclopropane-1-carbonitrile instead of 2-aminoethan-1-ol in Step 7. LCMS calculated for C₂₇H₃₀N₉O₂ (M+H)⁺ m/z=512.3; found 512.3. ¹H NMR (400 MHz, Methanol-d₄) δ 8.90 (s, 1H), 7.93 (d, J=8.4 Hz, 1H), 7.59-7.54 (m, 2H), 7.22 (d, J=1.2 Hz, 1H), 4.62 (s, 2H), 4.35 (q, J=7.2 Hz, 2H), 3.59-3.51 (m, 4H), 3.35-3.25 (m, 4H), 2.59 (s, 3H), 1.63-1.55 (m, 2H), 1.41-1.31 (m, 5H).

### Example 18. N-cyclobutyl-5-(4-((3-ethyl-2-oxo-2,3-dihydro-1H-pyrimido[4,5,6-de]quinazolin-8-yl)methyl)piperazin-1-yl)-6-methylpicolinamide

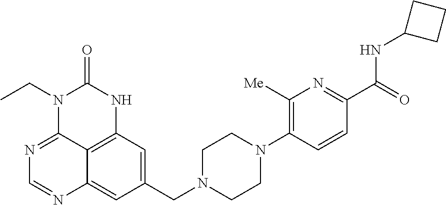 [SMILES: CCN1C(=O)Nc2cc(CN3CCN(c4ccc(C(=O)NC5CCC5)nc4C)CC3)cc3ncnc1c23]

The title compound was prepared according to the procedure described in Example 13, using cyclobutanamine instead of 2-aminoethan-1-ol in Step 7. LCMS calculated for C₂₇H₃₃N₈O₂ (M+H)⁺ m/z=501.3; found 501.4. ¹H NMR (400 MHz, Methanol-d₄) δ 8.81 (s, 1H), 7.89 (d, J=8.4 Hz, 1H), 7.56 (d, J=8.4 Hz, 1H), 7.52 (d, J=1.2 Hz, 1H), 7.13 (d, J=1.2 Hz, 1H), 4.59 (s, 2H), 4.55-4.44 (m, 1H), 4.32 (q, J=7.2 Hz, 2H), 3.60-3.46 (m, 4H), 3.31-3.21 (m, 4H), 2.61 (s, 3H), 2.40-2.30 (m, 2H), 2.20-2.07 (m, 2H), 1.85-1.73 (m, 2H), 1.33 (t, J=7.2 Hz, 3H).

### Example 19. 5-(4-((3-ethyl-2-oxo-2,3-dihydro-1H-pyrimido[4,5,6-de]quinazolin-8-yl)methyl)piperazin-1-yl)-N-((1s,3s)-3-hydroxycyclobutyl)-6-methylpicolinamide

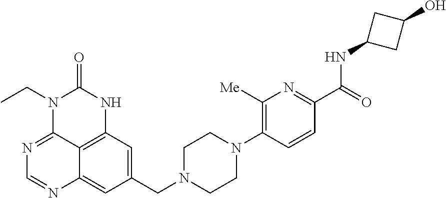 [SMILES: CCN1C(=O)Nc2cc(CN3CCN(c4ccc(C(=O)N[C@H]5C[C@@H](O)C5)nc4C)CC3)cc3ncnc1c23]

The title compound was prepared according to the procedure described in Example 13, using (1s,3s)-3-aminocyclobutan-1-ol instead of 2-aminoethan-1-ol in Step 7. LCMS calculated for C₂₇H₃₃N₈O₃ (M+H)⁺ m/z=517.3; found 517.3. ¹H NMR (400 MHz, Methanol-d₄) δ 8.82 (s, 1H), 7.89 (d, J=8.4 Hz, 1H), 7.56 (d, J=8.4 Hz, 1H), 7.52 (d, J=1.2 Hz, 1H), 7.13 (d, J=1.2 Hz, 1H), 4.59 (s, 2H), 4.32 (q, J=7.2 Hz, 2H), 4.09-3.98 (m, 2H), 3.59-3.47 (m, 4H), 3.31-3.22 (m, 4H), 2.80-2.71 (m, 2H), 2.61 (s, 3H), 2.05-1.94 (m, 2H), 1.33 (t, J=7.2 Hz, 3H).

### Example 20. 5-(4-((3-ethyl-2-oxo-2,3-dihydro-1H-pyrimido[4,5,6-de]quinazolin-8-yl)methyl)piperazin-1-yl)-N-((1r,3r)-3-hydroxycyclobutyl)-6-methylpicolinamide

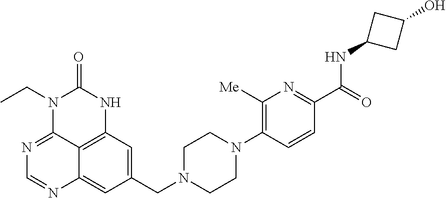 [SMILES: CCN1C(=O)Nc2cc(CN3CCN(c4ccc(C(=O)N[C@H]5C[C@H](O)C5)nc4C)CC3)cc3ncnc1c23]

The title compound was prepared according to the procedure described in Example 13, using (1r,3r)-3-aminocyclobutan-1-ol instead of 2-aminoethan-1-ol in Step 7. LCMS calculated for C₂₇H₃₃N₈O₃ (M+H)⁺ m/z=517.3; found 517.3. ¹H NMR (400 MHz, Methanol-d₄) δ 8.82 (s, 1H), 7.89 (d, J=8.4 Hz, 1H), 7.57 (d, J=8.4 Hz, 1H), 7.52 (d, J=1.2 Hz, 1H), 7.13 (d, J=1.2 Hz, 1H), 4.64-4.55 (m, 3H), 4.50-4.42 (m, 1H), 4.32 (q, J=7.2 Hz, 2H), 3.58-3.48 (m, 4H), 3.31-3.21 (m, 4H), 2.61 (s, 3H), 2.46-2.35 (m, 4H), 1.33 (t, J=7.2 Hz, 3H).

### Example 21. N-((1R,5S,6r)-3-oxabicyclo[3.1.0]hexan-6-yl)-5-(4-((3-ethyl-2-oxo-2,3-dihydro-1H-pyrimido[4,5,6-de]quinazolin-8-yl)methyl)piperazin-1-yl)-6-methylpicolinamide

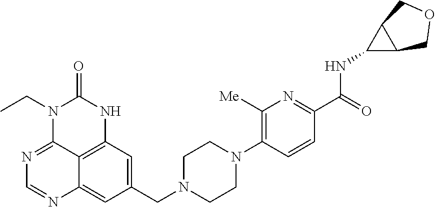 [SMILES: CCN1C(=O)Nc2cc(CN3CCN(c4ccc(C(=O)N[C@H]5[C@@H]6COC[C@@H]65)nc4C)CC3)cc3ncnc1c23]

The title compound was prepared according to the procedure described in Example 13, using (1R,5S,6r)-3-oxabicyclo[3.1.0]hexan-6-amine instead of 2-aminoethan-1-ol in Step 7. LCMS calculated for C₂₈H₃₃N₈O₃ (M+H)⁺ m/z=529.3; found 529.3. ¹H NMR (400 MHz, Methanol-d₄) δ 8.81 (s, 1H), 7.91 (d, J=8.4 Hz, 1H), 7.56 (d, J=8.4 Hz, 1H), 7.51 (d, J=1.2 Hz, 1H), 7.12 (d, J=1.2 Hz, 1H), 4.58 (s, 2H), 4.32 (q, J=7.2 Hz, 2H), 4.02 (d, J=8.4 Hz, 2H), 3.74 (d, J=8.4 Hz, 2H), 3.58-3.47 (m, 4H), 3.31-3.21 (m, 4H), 2.64-2.61 (m, 1H), 2.58 (s, 3H), 1.98-1.95 (m, 2H), 1.33 (t, J=7.2 Hz, 3H).

### Example 24. N-((1r,3r)-3-cyanocyclobutyl)-5-(4-((3-ethyl-2-oxo-2,3-dihydro-1H-pyrimido[4,5,6-de]quinazolin-8-yl)methyl)piperazin-1-yl)-6-methylpicolinamide

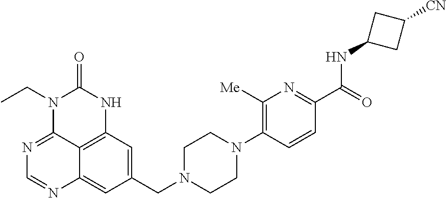 [SMILES: CCN1C(=O)Nc2cc(CN3CCN(c4ccc(C(=O)N[C@H]5C[C@H](C#N)C5)nc4C)CC3)cc3ncnc1c23]

The title compound was prepared according to the procedure described in Example 13, using (1r,3r)-3-aminocyclobutane-1-carbonitrile instead of 2-aminoethan-1-ol in Step 7. LCMS calculated for C₂₈H₃₂N₉O₂ (M+H)⁺ m/z=526.3; found 526.3; ¹H NMR (400 MHz, Methanol-d₄) δ 8.90 (s, 1H), 7.90 (d, J=8.4 Hz, 1H), 7.61-7.53 (m, 2H), 7.22 (d, J=1.2 Hz, 1H), 4.86-4.77 (m, 1H), 4.63 (s, 2H), 4.35 (q, J=7.2 Hz, 2H), 3.60-3.50 (m, 4H), 3.31-3.22 (m, 5H), 2.71-2.63 (m, 4H), 2.61 (s, 3H), 1.35 (t, J=7.2 Hz, 3H).

### Example 25. N-((1s,3s)-3-cyanocyclobutyl)-5-(4-((3-ethyl-2-oxo-2,3-dihydro-1H-pyrimido[4,5,6-de]quinazolin-8-yl)methyl)piperazin-1-yl)-6-methylpicolinamide

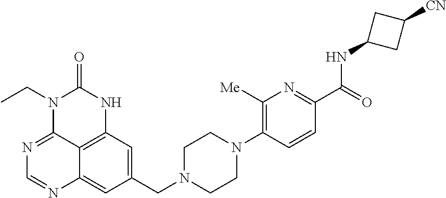 [SMILES: CCN1C(=O)Nc2cc(CN3CCN(c4ccc(C(=O)N[C@H]5C[C@@H](C#N)C5)nc4C)CC3)cc3ncnc1c23]

The title compound was prepared according to the procedure described in Example 13, using (1s,3s)-3-aminocyclobutane-1-carbonitrile instead of 2-aminoethan-1-ol in Step 7. LCMS calculated for C₂₈H₃₂N₉O₂ (M+H)⁺ m/z=526.3; found 526.3; ¹H NMR (400 MHz, Methanol-d₄) δ 8.91 (s, 1H), 7.89 (d, J=8.4 Hz, 1H), 7.60-7.54 (m, 2H), 7.24 (d, J=1.2 Hz, 1H), 4.63 (s, 2H), 4.59-4.48 (m, 1H), 4.36 (q, J=7.2 Hz, 2H), 3.59-3.51 (m, 4H), 3.32-3.23 (m, 4H), 3.10-2.98 (m, 2H), 2.84-2.72 (m, 2H), 2.62 (s, 3H), 2.60-2.54 (m, 1H), 1.35 (t, J=7.2 Hz, 3H).

### Example 26. N-(1-(cyanomethyl)cyclopropyl)-5-(4-((3-ethyl-2-oxo-2,3-dihydro-1H-pyrimido[4,5,6-de]quinazolin-8-yl)methyl)piperazin-1-yl)-6-methylpicolinamide

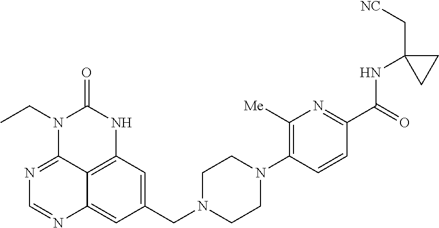 [SMILES: CCN1C(=O)Nc2cc(CN3CCN(c4ccc(C(=O)NC5(CC#N)CC5)nc4C)CC3)cc3ncnc1c23]

### Step 1: 1-(Bromomethyl)cyclopropan-1-amine

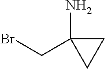

To the mixture of tert-butyl (1-(bromomethyl)cyclopropyl)carbamate (100 mg, 0.4 mmol) in dichloromethane (0.5 mL) was added trifluoroacetic acid (0.5 mL) at room temperature. The reaction mixture was stirred for 2 h. The resulting mixture was concentrated under reduced pressure to afford the TFA salt of the crude product which was used in the next step directly without further purification. LCMS calculated for C₄H₉BrN (M+H)⁺ m/z=150.0; found 150.0.

### Step 2: N-(1-(bromomethyl)cyclopropyl)-5-(4-((3-ethyl-2-oxo-2,3-dihydro-1H-pyrimido[4,5,6-de]quinazolin-8-yl)methyl)piperazin-1-yl)-6-methylpicolinamide

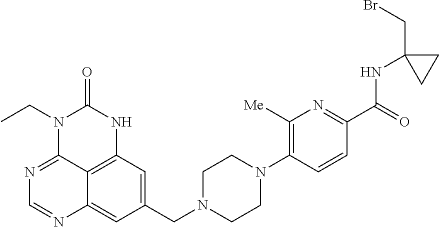 [SMILES: CCN1C(=O)Nc2cc(CN3CCN(c4ccc(C(=O)NC5(CBr)CC5)nc4C)CC3)cc3ncnc1c23]

The mixture of 5-(4-((3-ethyl-2-oxo-2,3-dihydro-1H-pyrimido[4,5,6-de]quinazolin-8-yl)methyl)piperazin-1-yl)-6-methylpicolinic acid (Example 13, Step 6: 50 mg, 0.11 mmol) and 2-(7-azabenzotriazol-1-yl)-N,N,N′,N′-tetramethyluronium hexafluorophosphate (64 mg, 0.17 mmol) in N,N-dimethylformamide (1 mL) was stirred for 0.5 h at room temperature. To the above mixture was added 1-(bromomethyl)cyclopropan-1-amine (25 mg, 0.17 mmol) and N,N-diisopropylethylamine (72 mg, 0.56 mmol). The reaction mixture was stirred for 2 h at room temperature. The mixture was purified by reversed-phase flash chromatography with the following conditions (Column: C18 silica gel; Mobile phase: acetonitrile in water (10 mmol/L ammonium bicarbonate); Gradient: 30% to 50% in 20 min; Detector: 254 nm). Eluted fractions were collected and concentrated to afford the desired product as a light-yellow solid (33 mg, 51%). LCMS calculated for C₂₇H₃₂BrN₈O₂(M+H)⁺ m/z=579.2; found 579.0.

### Step 3: N-(1-(cyanomethyl)cyclopropyl)-5-(4-((3-ethyl-2-oxo-2,3-dihydro-1H-pyrimido[4,5,6-de]quinazolin-8-yl)methyl)piperazin-1-yl)-6-methylpicolinamide

The mixture of N-(1-(bromomethyl)cyclopropyl)-5-(4-((3-ethyl-2-oxo-2,3-dihydro-1H-pyrimido[4,5,6-de]quinazolin-8-yl)methyl)piperazin-1-yl)-6-methylpicolinamide (30 mg, 0.05 mmol) and potassium cyanide (10 mg, 0.16 mmol) in dimethyl sulfoxide (2 mL) was stirred for 3 h at 40° C. Upon cooling to room temperature, the resulting mixture was diluted with water (30 mL). The resulting mixture was extracted with ethyl acetate (3×30 mL). The combined organic layers were washed with brine (60 mL), dried over anhydrous sodium sulfate. After filtration, the filtrate was concentrated under reduced pressure. The crude product was purified by Prep-HPLC with the following conditions (Column: Xselect CSH F-Phenyl OBD column 30*250 mm, 5 m; Mobile Phase: acetonitrile in water (0.05% trifluoroacetic acid); Flow rate: 60 mL/min; Gradient: 5% B to 23% B in 10 min; Detector: 254/220 nm). Eluted fractions were collected and lyophilized to afford the TFA salt of the desired product as a white solid. LCMS calculated for C₂₈H₃₂N₉O₂ (M+H)⁺ m/z=526.3; found 526.3; ¹H NMR (400 MHz, Methanol-d₄) δ 8.79 (s, 1H), 7.92 (d, J=8.4 Hz, 1H), 7.56 (d, J=8.4 Hz, 1H), 7.50 (d, J=1.2 Hz, 1H), 7.08 (d, J=1.2 Hz, 1H), 4.57 (s, 2H), 4.31 (q, J=7.2 Hz, 2H), 3.58-3.48 (m, 4H), 3.31-3.19 (m, 4H), 2.95 (s, 2H), 2.60 (s, 3H), 1.33 (t, J=7.2 Hz, 3H), 1.07-0.97 (m, 4H).

### Example 31. N-cyclopropyl-5-(4-((3-cyclopropyl-2-oxo-2,3-dihydro-1H-pyrimido[4,5,6-de]quinazolin-8-yl)methyl)piperazin-1-yl)-6-methylpicolinamide

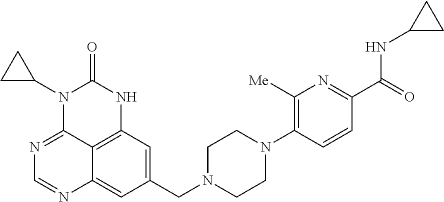 [SMILES: Cc1nc(C(=O)NC2CC2)ccc1N1CCN(Cc2cc3c4c(ncnc4c2)N(C2CC2)C(=O)N3)CC1]

### Step 1: 8-Bromo-3-cyclopropyl-1-(4-methoxybenzyl)-1H-pyrimido[4,5,6-de]quinazolin-2(3H)-one

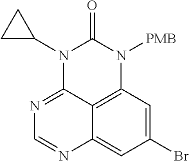

To a screw-cap vial equipped with a magnetic stir bar were placed 8-bromo-1-(4-methoxybenzyl)-1H-pyrimido[4,5,6-de]quinazolin-2(3H)-one (Example 13, Step 1: 1 g, 2.6 mmol), cyclopropylboronic acid (0.45 g, 5.2 mmol), cupric acetate (0.94 g, 5.2 mmol) and pyridine (0.41 g, 5.2 mmol). The vial was sealed with a Teflon-lined septum, evacuated and backfilled with oxygen (this process was repeated a total of three times). 1,2-Dichloroethane (10 mL) was added. The reaction was stirred at 80° C. for 16 h. Upon cooling to room temperature, the mixture was diluted with water (30 mL). The aqueous layer was extracted with dichloromethane (3×30 mL). The resulting mixture was concentrated under reduced pressure. The residue was purified by silica gel column chromatography, eluted with 50% ethyl acetate in petroleum ether to afford the desired product as a white solid (130 mg, 12%). LCMS calculated for C₂₀H₁₈BrN₄O₂(M+H)⁺ m/z=425.1; found 425.1.

### Step 2: 3-Cyclopropyl-8-(hydroxymethyl)-1-(4-methoxybenzyl)-1H-pyrimido[4,5,6-de]quinazolin-2(3H)-one

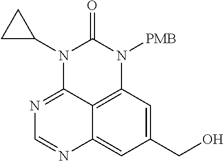

To a screw-cap vial equipped with a magnetic stir bar were placed 8-bromo-3-cyclopropyl-1-(4-methoxybenzyl)-1H-pyrimido[4,5,6-de]quinazolin-2(3H)-one (130 mg, 0.3 mmol), (tributylstannyl)methanol (196 mg, 0.6 mmol) and XPhos Pd G3 (52 mg, 0.06 mmol). The vial was sealed with a Teflon-lined septum, evacuated and backfilled with nitrogen (this process was repeated a total of three times). 1,4-Dioxane (3 mL) was added. The reaction mixture was stirred at 100° C. for 2 h. Upon cooling to room temperature, the resulting mixture was concentrated under reduced pressure. The residue was purified by silica gel column chromatography, eluted with 10% methanol in dichloromethane to afford the desired product as a white solid (80 mg, 69%). LCMS calculated for C₂₁H₂₁N₄O₃ (M+H)⁺ m/z=377.2; found 377.2.

### Step 3: N-cyclopropyl-5-(4-((3-cyclopropyl-2-oxo-2,3-dihydro-1H-pyrimido[4,5,6-de]quinazolin-8-yl)methyl)piperazin-1-yl)-6-methylpicolinamide

To 3-cyclopropyl-8-(hydroxymethyl)-1-(4-methoxybenzyl)-1H-pyrimido[4,5,6-de]quinazolin-2(3H)-one (60 mg, 0.16 mmol) was added hydrogen bromide (33 wt. % solution in glacial acid, 2 mL) at room temperature. The reaction mixture was then stirred at 80° C. for 16 h. Upon cooling to room temperature, the reaction mixture was concentrated under reduced pressure. The residue was taken in acetonitrile (3 mL), followed by the addition of N-cyclopropyl-6-methyl-5-(piperazin-1-yl)picolinamide (Intermediate 2: 46 mg, 0.18 mmol) and N,N-diisopropylethylamine (207 mg, 1.6 mmol). The resulting mixture was stirred at room temperature for 16 h, and then concentrated under reduced pressure. The residue was purified by reversed-phase flash chromatography with the following conditions (Column: C18 silica gel; Mobile Phase: acetonitrile in water (0.1% trifluoroacetic acid); Gradient: 5% B to 95% B in 30 min; Detector: 254/220 nm). Eluted fractions were lyophilized to afford the TFA salt of the desired product as a white solid. LCMS calculated for C₂₇H₃₁N₈O₂ (M+H)⁺ m/z=499.3; found 499.3; ¹H NMR (400 MHz, DMSO-d₆) δ 11.60 (s, 1H), 8.80 (s, 1H), 8.37 (d, J=4.8 Hz, 1H), 7.83 (d, J=8.4 Hz, 1H), 7.55 (d, J=8.4 Hz, 1H), 7.48 (d, J=1.2 Hz, 1H), 6.97 (d, J=1.2 Hz, 1H), 4.57 (s, 2H), 3.51-3.25 (m, 6H), 3.08-2.94 (m, 2H), 2.91-2.80 (m, 1H), 2.79-2.72 (m, 1H), 2.50 (s, 3H), 1.18-1.10 (m, 2H), 0.88-0.79 (m, 2H), 0.75-0.67 (m, 2H), 0.67-0.60 (m, 2H).

### Example 32. 5-(4-((3-(Cyanomethyl)-2-oxo-2,3-dihydro-1H-pyrimido[4,5,6-de]quinazolin-8-yl)methyl)piperazin-1-yl)-N-cyclopropyl-6-methylpicolinamide

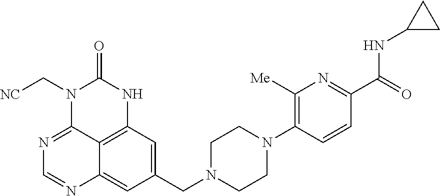 [SMILES: Cc1nc(C(=O)NC2CC2)ccc1N1CCN(Cc2cc3c4c(ncnc4c2)N(CC#N)C(=O)N3)CC1]

### Step 1: 2-(8-Bromo-1-(4-methoxybenzyl)-2-oxo-1,2-dihydro-3H-pyrimido[4,5,6-de]quinazolin-3-yl)acetonitrile

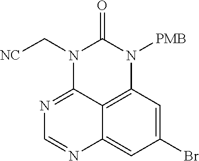

To the mixture of 8-bromo-1-(4-methoxybenzyl)-1H-pyrimido[4,5,6-de]quinazolin-2(3H)-one (200 mg, 0.52 mmol) and potassium carbonate (Example 13, Step 1: 143 mg, 1.0 mmol) in N,N-dimethylformamide (3 mL) was added iodoacetonitrile (104 mg, 0.6 mmol) dropwise at 0° C. The reaction mixture was stirred at room temperature for 2 h. The mixture was diluted with water (20 mL). The aqueous layer was extracted with ethyl acetate (3×20 mL). The resulting mixture was concentrated under reduced pressure. The residue was purified by silica gel column chromatography, eluted with 50% ethyl acetate in petroleum ether to afford the desired product as a yellow solid (210 mg, 95%). LCMS calculated for C₁₉H₁₅BrN₅O₂ (M+H)⁺ m/z=424.0; found 424.1.

### Step 2: 2-(8-Bromo-2-oxo-1,2-dihydro-3H-pyrimido[4,5,6-de]quinazolin-3-yl)acetonitrile

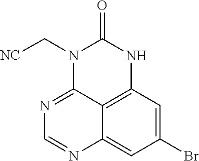 [SMILES: N#CCN1C(=O)Nc2cc(Br)cc3ncnc1c23]

To the mixture of 2-(8-bromo-1-(4-methoxybenzyl)-2-oxo-1,2-dihydro-3H-pyrimido[4,5,6-de]quinazolin-3-yl)acetonitrile (500 mg, 1.18 mmol) in acetonitrile (5 mL) and water (5 mL) was added diammonium cerium(IV) nitrate (1.95 g, 3.54 mmol) at room temperature. After stirring at room temperature for 16 h, the reaction mixture was diluted with water (50 mL). The aqueous layer was extracted with dichloromethane (3×100 mL), dried over anhydrous sodium sulfate. After filtration, the filtrate was concentrated under reduced pressure. The residue was purified by silica gel column chromatography, eluted with 75% ethyl acetate in petroleum ether to afford the desired product as a yellow solid (100 mg, 28%). LCMS calculated for C₁H₅BrN₅O (M−H)⁻ m/z=302.0; found 302.0.

### Step 3: 2-(8-(Hydroxymethyl)-2-oxo-1,2-dihydro-3H-pyrimido[4,5,6-de]quinazolin-3-yl)acetonitrile

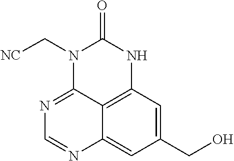 [SMILES: N#CCN1C(=O)Nc2cc(CO)cc3ncnc1c23]

To a screw-cap vial equipped with a magnetic stir bar were placed 2-(8-bromo-2-oxo-1,2-dihydro-3H-pyrimido[4,5,6-de]quinazolin-3-yl)acetonitrile (100 mg, 0.33 mmol), (tributylstannyl)methanol (158 mg, 0.5 mmol) and XPhos Pd G3 (55 mg, 0.07 mmol). The vial was sealed with a Teflon-lined septum, evacuated and backfilled with nitrogen (this process was repeated a total of three times). 1,4-Dioxane (3 mL) was added. The reaction mixture was stirred at 100° C. for 1 h. Upon cooling to room temperature, the resulting mixture was concentrated under reduced pressure. The residue was purified by silica gel column chromatography, eluted with 10% methanol in dichloromethane to afford the desired product as a white solid (50 mg, 59%). LCMS calculated for C₁₂H₁₀N₅O₂(M+H)⁺ m/z=256.1; found 256.1.

### Step 4: 2-(8-Formyl-2-oxo-1,2-dihydro-3H-pyrimido[4,5,6-de]quinazolin-3-yl)acetonitrile

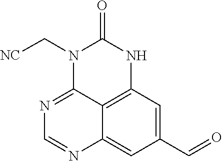 [SMILES: N#CCN1C(=O)Nc2cc(C=O)cc3ncnc1c23]

To the mixture of 2-(8-(Hydroxymethyl)-2-oxo-1,2-dihydro-3H-pyrimido[4,5,6-de]quinazolin-3-yl)acetonitrile (45 mg, 0.18 mmol) in dichloromethane (2 mL) were added Dess-Martin periodinane (374 mg, 0.88 mmol) and sodium bicarbonate (30 mg, 0.35 mmol) at room temperature. After stirring at room temperature for 6 h, the reaction mixture was concentrated under reduced pressure. The residue was purified by Prep-TLC (10% methanol in dichloromethane) to afford the desired product as a yellow solid (10 mg, 22%). LCMS calculated for C₁₂H₈N₅O₂(M+H)⁺ m/z=254.1; found 254.1.

### Step 5: 5-(4-((3-(Cyanomethyl)-2-oxo-2,3-dihydro-1H-pyrimido[4,5,6-de]quinazolin-8-yl)methyl)piperazin-1-yl)-N-cyclopropyl-6-methylpicolinamide

The mixture of 2-(8-formyl-2-oxo-1,2-dihydro-3H-pyrimido[4,5,6-de]quinazolin-3-yl)acetonitrile (10 mg, 0.04 mmol), N-cyclopropyl-6-methyl-5-(piperazin-1-yl)picolinamide (Intermediate 2: 12 mg, 0.05 mmol) and acetic acid (7 mg, 0.12 mmol) in methanol (1 mL) was stirred at room temperature for 30 min. To the above mixture was added sodium cyanoborohydride (5 mg, 0.08 mmol) at 0° C. After stirring at room temperature for 16 h, the reaction mixture was purified by reversed-phase flash chromatography with the following conditions (Column: C18 silica gel; Mobile phase: acetonitrile in water (0.1% trifluoroacetic acid), Gradient: 10% to 50% in 25 min; Detector: 254 nm). Eluted fractions were collected and lyophilized to afford the TFA salt of the desired product as a white solid. LCMS calculated for C₂₆H₂₈N₉O₂ (M+H)⁺ m/z=498.2; found 498.3; ¹H NMR (400 MHz, Methanol-d₄) δ 8.86 (s, 1H), 7.91 (d, J=8.4 Hz, 1H), 7.60 (d, J=1.2 Hz, 1H), 7.57 (d, J=8.4 Hz, 1H), 7.13 (d, J=1.2 Hz, 1H), 5.20 (s, 2H), 4.61 (s, 2H), 3.62-3.49 (m, 4H), 3.32-3.18 (m, 4H), 2.89-2.81 (m, 1H), 2.58 (s, 3H), 0.86-0.80 (m, 2H), 0.70-0.62 (m, 2H).

### Example A. In-Cell Western Measurement of PARylation in PARP1 WT and Knockout Cells

The purpose of this cell-based assay is to measure the potency and selectivity of synthesized PARP inhibitors in suppressing hydrogen peroxide-induced PARylation in cells with and without PARP1 expression. Several cell lines were used in this assay. HeLa cells (wide-type with PARP1 gene) were purchased from ATCC. The HAP1 cell line without the PARP1 gene (#HZGHC003943c006) was purchased from Horizon Discovery. Cells were cultured using complete medium containing 10% fetal bovine serum. One day before the assay, cells were seeded in 96-well plates at the density of 20,000 cells per well in 80 uL complete medium. Compounds were dissolved using DMSO and diluted to the working concentrations using complete medium. Cells were pre-treated with compounds for 60 min and then stimulated with 10 mM hydrogen peroxide for 15 min. After stimulation, cells were immediately fixed using ice-cold methanol for 20 min. After fixation, cells were washed using 1×PBST buffer for 3 times, 5 min each time, to eliminate residual methanol. Cells were blocked for 1 h at room temperature in blocking buffer (10% goat serum, 1% BSA, 0.1% Triton X-100 in PBST). Primary antibody was diluted in blocking buffer at 50 uL per well. Cells were incubated with the primary antibody (Adipogen, AG-20T-0001-M001, 1:300) at 4° C. for 18 h. The wells were then washed using 1×PBST for 3 times, 5 min each time. IRDye® 680RD Goat anti-Mouse IgG (H+L) secondary antibody was diluted at 1/1000 using the diluent buffer and added at 50 uL per well. Secondary antibody incubation was for 1 h at room temperature. Cells were then washed using 1×PBST for 3 times, 5 min each time. The signal was detected and analyzed using LI-COR Odyssey DLx Imaging system. Data were collected and further processed using GraphPad Prism for IC₅₀ estimation.

Results of the assay described above for HeLa_WT are presented in Table 1. Compounds denoted of the present disclosure showed IC₅₀ values in the following ranges:
A: IC₅₀≤10 nM;
B: 10 nM<IC₅₀≤100 nM;
C: 100 nM<IC₅₀≤500 nM;
D: 500 nM<IC₅₀≤1,000 nM;
E: 1,000 nM<IC₅₀≤10,000 nM.

TABLE 1

| Compound | HeLa_WT_IC₅₀ (nM) |
|---|---|
| 1 | B |
| 2 | B |
| 3 | A |
| 4 | A |
| 5 | A |
| 6 | A |
| 7 | A |
| 8 | A |
| 9 | A |
| 10 | A |
| 13 | A |
| 14 | A |
| 15 | A |
| 16 | A |
| 17 | A |
| 18 | A |
| 19 | A |
| 20 | A |
| 21 | A |
| 24 | A |
| 25 | A |
| 26 | A |

### Example B. Cell-Titer Glo Measurement of Cytotoxicity in BRCA2 Isogenic Cells

The purpose of this cellular assay is to measure cytotoxicity and cell killing activity of selected PARP1 inhibitors in DLD1 isogenic cells with and without the BRCA2 genes. The BRCA2 knockout DLD1 cell line (#HD 105-007) and its isogenic wide-type control were purchased from Horizon Discovery. Cells were cultured using RPMI 1640 complete medium containing 10% fetal bovine serum and Penicillin-Streptomycin. One day before the assay, cells were seeded at 50-750 cells per well in 96-well plates. Compounds were dissolved and diluted in DMSO and added into complete medium to final working concentrations. The treatment was for 7 days. At the end of this assay, Promega Cell-titer Glo reagents (#G7573) were added at 100 uL per well. Plates were kept on orbital shaker for 2 min. These plates were then kept in the CO2 incubator for another 10 minutes. Luminescence signal was measured using the SpectraMax i3× plate reader. Data were further analyzed using GraphPad Prism for IC₅₀ estimation.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula I: 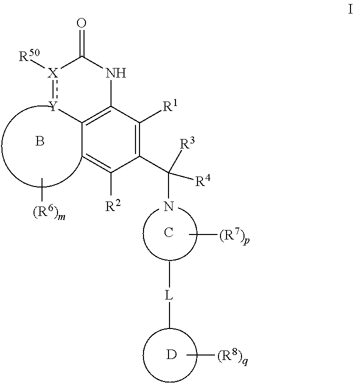
or a pharmaceutically acceptable salt thereof, wherein:
X is C or N;
Y is C or N; [symbol]
is a single or double bond;
m is 0, 1, 2, 3, 4, 5, or 6;
p is 0, 1, 2, 3, 4, 5, or 6;
q is 0, 1, 2, 3, 4, 5, or 6;
Ring B is C₅₋₁₀ cycloalkyl, phenyl, 5-10 membered heterocycloalkyl, or 5-6 membered heteroaryl;
Ring C is 4-14 membered heterocycloalkyl;
Ring D is C₃₋₁₀ cycloalkyl, C₆₋₁₀ aryl, 4-10 membered heterocycloalkyl, or 5-10 membered heteroaryl;
L is selected from bond, C₁₋₄ alkylene, C₁₋₄ haloalkylene, C₃₋₇ cycloalkylene, 4-7 membered heterocycloalkylene, —C₃₋₇ cycloalkylene-C₁₋₄ alkyl-, -(4-7 membered heterocycloalkylene)-C₁₋₄ alkyl-, —O—, —N(R^{L})—, —C(O)—, —C(O)N(R^{L})—, —N(R^{L})C(O)N(R^{L})—, —N(R^{L})C(O)O—, —S(O)₂—, —N(R^{L})S(O)₂—, and —N(R^{L})S(O)₂N(R^{L})—, wherein the C₁₋₄ alkylene, C₁₋₄ haloalkylene, C₃₋₇ cycloalkylene, 4-7 membered heterocycloalkylene, C₃₋₇ cycloalkylene-C₁₋₄ alkyl, and (4-7 membered heterocycloalkylene)-C₁₋₄ alkyl of L are each optionally substituted with 1, 2, 3, or 4 independently selected R^{G} substituents;
each R^{L} is independently selected from H, C₁₋₆ alkyl, and C₁₋₆ haloalkyl;
R¹ is selected from H, halo, CN, C₁₋₃ alkyl, C₁₋₃ haloalkyl, C₁₋₃ alkoxy, and C₁₋₃ haloalkoxy;
R² is selected from H, halo, CN, C₁₋₃ alkyl, C₁₋₃ haloalkyl, C₁₋₃ alkoxy, and C₁₋₃ haloalkoxy;
R³ is selected from H, halo, C₁₋₃ alkyl, and C₁₋₃ haloalkyl;
R⁴ is selected from H, halo, C₁₋₃ alkyl, and C₁₋₃ haloalkyl;
or, R³ and R⁴, together with the carbon atom to which they are attached, form a C₃₋₇ cycloalkyl, or 4-7 membered heterocycloalkyl group, wherein the C₃₋₇ cycloalkyl, and 4-7 membered heterocycloalkyl group is optionally substituted with 1, 2, 3, 4, 5, or 6 independently selected R^{G} substituents;
R⁵⁰ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a50}, —SR^{a50}, —NR^{c50}R^{d50}, —NO₂, —C(O)R^{a50}, —C(O)OR^{a50}, —C(O)NR^{c50}R^{d50}, —C(O)NR^{c50}(OR^{a50}), —OC(O)R^{a50}, —OC(O)NR^{c50}R^{d50}, —OC(O)OR^{a50}, —OS(O)₂R^{b50}, —OS(O)₂NR^{c50}R^{d50}, —NR^{c50}C(O)R^{a50}, —NR^{c50}C(O)OR^{a50}, —NR^{c50}C(O)NR^{c50}R^{d50}, —NR^{c50}S(O)₂R^{b50}, —NR^{c50}S(O)₂NR^{c50}R^{d50}, —NR^{c50}OR^{a50}, —NR^{c50}S(O)R^{b50}, —NR^{c50}S(O)NR^{c50}R^{d50}, —S(O)R^{b50}, —S(O)₂R^{b50}, —S(O)NR^{c50}R^{d50}, —S(O)₂NR^{c50}R^{d50}, —C(═NR^{e50})R^{a50}, —C(═NR^{e50})NR^{c50}R^{d50}, —NR^{c50}C(═NR^{e50})R^{a50}, —NR^{c50}C(═NR^{e50})NR^{c50}R^{d50}, —NR^{c50}S(O)(═NR^{e50})R^{b50}, —NR^{c50}S(O)(═NR^{e50})NR^{c50}R^{d50}, —OS(O)(═NR^{e50})R^{b50}, —S(O)(═NR^{e50})R^{b50}, —S(O)(═NR^{e50})NR^{c50}R^{d50}, —C(O)NR^{c50}S(O)₂R^{b50}, —C(O)NR^{c50}S(O)₂NR^{c50}R^{d50}, —S(O)₂NR^{c50}C(O)R^{b50}, —NR^{c50}S(O)NR^{c50}C(O)R^{b5}, and —P(O)R^{f50}R^{g50}, wherein the C₁₋₆ alkyl, C₂₋₆ alkenyl, C₂-6 alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁵⁰ are each optionally substituted with 1, 2, 3, 4, 5, or 6 independently selected R^{G} substituents;
each R^{a50}, R^{c50}, and R^{d50} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a50}, R^{c50} and R^{d50} are each optionally substituted with 1, 2, 3, 4, 5, or 6 independently selected R^{G} substituents;
or, any R^{c50} and R^{d50} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b50} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b50} are each optionally substituted with 1, 2, 3, 4, 5, or 6 independently selected R^{G} substituents;
each R^{e50} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f50} and R^{g50} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
or, R⁵⁰ and one R⁶, taken together with the atoms to which they are attached, form a Ring A which is selected from C₅₋₁₀ cycloalkyl, phenyl, 5-10 membered heterocycloalkyl, and 5-6 membered heteroaryl, wherein the C₅₋₁₀ cycloalkyl, phenyl, 5-10 membered heterocycloalkyl, and 5-6 membered heteroaryl group is optionally substituted with 1, 2, 3, 4, 5, or 6 independently selected R⁵ substituents;
each R⁵ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{c5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R^{b5}, —NR^{c5}S(O)₂NR^{c5}R^{d5}, —NR^{c5}R^{d5}, —NR^{c5}S(O)R^{b5}, —NR^{c5}S(O)NR^{c5}R^{d5}, —S(O)R^{b5}, —S(O)₂R^{c5}, —S(O)NR^{c5}R^{d5}, —S(O)₂NR^{c5}R^{d5}, —C(═NR^{e5})R^{a5}, —C(═NR^{e5})NR^{c5}R^{d5}, —NR^{c5}C(═NR^{e5})R^{a5}, —NR^{c5}C(═NR^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g5}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁵ are each optionally substituted with 1, 2, 3, 4, 5, or 6 independently selected R^{G} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a5}, R^{c5}, and R^{d5} are each optionally substituted with 1, 2, 3, 4, 5, or 6 independently selected R^{G} substituents;
or, any R^{a5} and R^{d5} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b5} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b5} are each optionally substituted with 1, 2, 3, 4, 5, or 6 independently selected R^{G} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁶ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6}, —SR^{a6}, —NR^{c6}R^{d6}, —NO₂, —C(O)R^{a6}, —C(O)OR^{a6}, —C(O)NR^{c6}R^{d6}, —C(O)NR^{c6}(OR^{a6}), —OC(O)R^{a6}, —OC(O)NR^{c6}R^{d6}, —OC(O)OR^{a6}, —OS(O)₂R^{b6}, —OS(O)₂NR^{c6}R^{d6}, —NR^{c6}C(O)R^{a6}, —NR^{c6}C(O)OR^{a6}, —NR^{c6}C(O)NR^{c6}R^{d6}, —NR^{c6}S(O)₂R^{b6}, —NR^{c6}S(O)₂NR^{c6}R^{d6}, —NR^{c6}OR^{a6}, —NR^{c6}S(O)R^{b6}, —NR^{c6}S(O)NR^{c6}R^{d6}, —S(O)R^{b6}, —S(O)₂R^{b6}, —S(O)NR^{c6}R^{d6}, —S(O)₂NR^{c6}R^{d6}, —C(═NR^{e6})R^{a6}, —C(═NR^{e6})R^{c6}R^{d6}, —R^{c6}C(═NR^{e6})R^{a6}, —NR^{c6}C(═NR^{e6})NR^{c6}R^{d6}, —NR^{c6}S(O)(═NR^{e6})R^{b6}, —NR^{c6}S(O)(═NR^{e6})NR^{c6}R^{d6}, —OS(O)(═NR^{e6})R^{b6}, —S(O)(═NR^{e6})R^{b6}, —S(O)(═NR^{e6})NR^{c6}R^{d6}, —C(O)NR^{c6}S(O)₂R^{b6}, —C(O)NR^{c6}S(O)₂NR^{c6}R^{d6}, —S(O)₂NR^{c6}C(O)R^{b6}, —NR^{c6}S(O)NR^{c6}C(O)R^{b6}, and —P(O)R^{f6}R^{g6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, 4, 5, or 6 independently selected R^{G} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a6}, R^{c6}, and R^{d6} are each optionally substituted with 1, 2, 3, 4, 5, or 6 independently selected R^{G} substituents;
or, any R^{c6} and R^{d6} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b6} are each optionally substituted with 1, 2, 3, 4, 5, or 6 independently selected R^{G} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f6} and R^{g6} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁷ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a7}, —SR^{a7}, —NR^{c7}R^{d7}, —NO₂, —C(O)R^{a7}, —C(O)OR^{a7}, —C(O)NR^{c7}R^{d7}, —C(O)NR^{c7}(OR^{a7}), —OC(O)R^{a7}, —OC(O)NR^{c7}R^{d7}, —OC(O)OR^{a7}, —OS(O)₂R^{b7}, —OS(O)₂NR^{c7}R^{d7}, —NR^{c7}C(O)R^{a7}, —NR^{c7}C(O)OR^{a7}, —NR^{c7}C(O)NR^{c7}R^{d7}, —NR^{c7}S(O)₂R^{b7}, —NR^{c7}S(O)₂NR^{c7}R^{d7}, —NR^{c7}OR^{a7}, —NR^{c7}S(O)R^{b7}, —R^{c7}S(O)NR^{c7}R^{d7}, —S(O)R^{b7}, —S(O)₂R^{b7}, —S(O)NR^{c7}R^{d7}, —S(O)₂NR^{c7}R^{d7}, —C(═NR^{e7})R^{a7}, —C(═NR^{e7})NR^{c7}R^{d7}, —NR^{c7}C(═NR^{e7})R^{a7}, —NR^{c7}C(═NR^{c7})NR^{c7}R^{d7}, —NR^{c7}S(O)(═NR^{e7})R^{b7}, —NR^{c7}S(O)(═NR^{e7})NR^{c7}R^{d7}, —OS(O)(═NR^{e7})R^{b7}, —S(O)(═NR^{e7})R^{b7}, —S(O)(═NR^{e7})NR^{c7}R^{d7}, —C(O)NR^{c7}S(O)₂R^{b7}, —C(O)NR^{c7}S(O)₂NR^{c7}R^{d7}, —S(O)₂NR^{c7}C(O)R^{b7}, —NR^{c7}S(O)NR^{c7}C(O)R^{b7} and —P(O)R^{f7}R^{g7}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁷ are each optionally substituted with 1, 2, 3, 4, 5, or 6 independently selected R^{G} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a7}, R^{c7}, and R^{d7} are each optionally substituted with 1, 2, 3, 4, 5, or 6 independently selected R^{G} substituents;
or, any R^{c7} and R^{d7} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{b7} are each optionally substituted with 1, 2, 3, 4, 5, or 6 independently selected R^{G} substituents;
each R^{c7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁸ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, —C(═NR^{e8})R^{a8}, —C(═NR^{e8})NR^{c8}R^{d8}, —NR^{c8}C(═NR^{e8})R^{a8}, —NR^{c8}C(═NR^{e8})NR^{c8}R^{d8}, —NR^{c8}S(O)(═NR^{e8})R^{b8}, —NR^{c8}S(O)(═NR^{e8})NR^{c8}R^{d8}, —OS(O)(═NR^{e8})R^{b8}, —S(O)(═NR^{e8})R^{b8}, —S(O)(═NR^{e8})NR^{c8}R^{d8}, —C(O)NR^{c8}S(O)₂R^{b8}, —C(O)NR^{c8}S(O)₂NR^{c8}R^{d8}, —S(O)₂NR^{c8}C(O)R^{b8}, —NR^{c8}S(O)NR^{c8}C(O)R^{b8}, and —P(O)R^{f8}R^{g8}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁸ are each optionally substituted with 1, 2, 3, 4, 5, or 6 independently selected R^{8A} substituents;
or, a R⁷ and a R⁸, together with the atoms to which they are attached, form a C₅₋₁₀ cycloalkyl, phenyl, 5-10 membered heterocycloalkyl, or 5-6 membered heteroaryl group, wherein the C₅₋₁₀ cycloalkyl, phenyl, 5-10 membered heterocycloalkyl, or 5-6 membered heteroaryl group is optionally substituted with 1, 2, 3, 4, 5, or 6 independently selected R^{8A} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{c8}, and R^{d8} are each optionally substituted with 1, 2, 3, 4, 5, or 6 independently selected R^{8A} substituents;
or, any R^{c8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{b8} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8} are each optionally substituted with 1, 2, 3, 4, 5, or 6 independently selected R^{8A} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{c8A}R^{d8A}, —NO₂, —C(O)R^{a8A}, —C(O)OR^{a8A}, —C(O)NR^{c8A}R^{d8A}, —C(O)NR^{c8A}(OR^{a8A}), —OC(O)R^{a8A}, —OC(O)NR^{c8A}R^{d8A}, —OC(O)OR^{a8A}, —OS(O)₂R^{b8A}, —OS(O)₂NR^{c8A}R^{d8A}, —NR^{c8A}C(O)R^{a8A}, —NR^{c8A}C(O)OR^{a8A}, —NR^{c8A}C(O)NR^{c8A}R^{d8A}, —NR^{c8A}S(O)₂R^{b8A}, —NR^{c8A}S(O)₂NR^{c8A}R^{d8A}, —NR^{e8A}OR^{a8A}, —NR^{c8A}S(O)R^{b8A}, —NR^{c8A}S(O)NR^{c8A}R^{d8A}, —S(O)R^{b8A}, —S(O)₂R^{b8A}, —S(O)NR^{c8A}R^{d8A}, —S(O)₂NR^{c8A}R^{d8A}, C(═NR^{c8A})R^{b8A}, —C(═NR^{c8A})R^{c8A}R^{d8A}, —NR^{c8A}C(═NR^{c8A})R^{a8A}, —NR^{c8A}C(═NR^{c8A})NR^{c8A}R^{d8A}, —NR^{e8A}S(O)(═NR^{c8A})R^{b8A}, —NR^{e8A}S(O)(═NR^{c8A})NR^{c8A}R^{d8A}, —OS(O)(═NR^{c8A})R^{b8A}, —S(O)(═NR^{c8A})R^{b8A}, —S(O)(═NR^{c8A})R^{c8A}R^{d8A}, —C(O)NR^{e8A}S(O)₂R^{b8A}, —C(O)NR^{c8A}S(O)₂NR^{c8A}R^{d8A}, —S(O)₂NR^{c8A}C(O)R^{b8A}, —NR^{e8A}S(O)NR^{c8A}C(O)R^{b8A}, and —P(O)R^{f8A}R^{g8A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{8A} are each optionally substituted with 1, 2, 3, 4, 5, or 6 independently selected R^{G} substituents;
each R^{a8A}, R^{c8A}, and R^{d8A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8A}, R^{c8A} and R^{d8A} are each optionally substituted with 1, 2, 3, 4, 5, or 6 independently selected R^{G} substituents;
or, any R^{b8A} and R^{d8A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b8A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8A} are each optionally substituted with 1, 2, 3, 4, 5, or 6 independently selected R^{G} substituents;
each R^{c8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2. The compound of claim 1, wherein the compound of Formula I is a compound of Formula II: 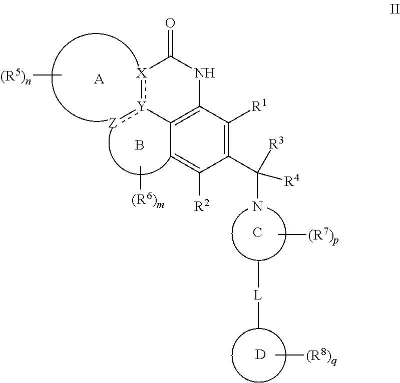
or a pharmaceutically acceptable salt thereof, wherein:
Z is C or N; and
each [symbol] is independently a single or double bond.

3. The compound of claim 1 or 2, or a pharmaceutically acceptable salt thereof, wherein X is N.

4. The compound of any one of claims 1 to 3, or a pharmaceutically acceptable salt thereof, wherein Y is C.

5. The compound of any one of claims 2 to 4, or a pharmaceutically acceptable salt thereof, wherein Z is N.

6. The compound of any one of claims 2 to 4, or a pharmaceutically acceptable salt thereof, wherein Z is C.

7. The compound of claim 1 or 2, or a pharmaceutically acceptable salt thereof, wherein Ring A is selected from

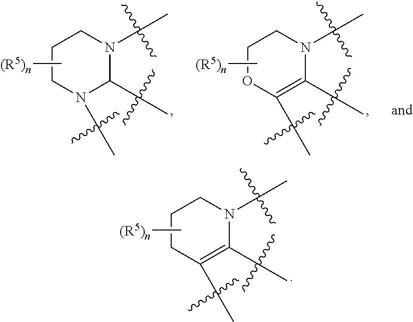

8. The compound of any one of claims 2 to 7, or a pharmaceutically acceptable salt thereof, wherein n is 0 or 1.

9. The compound of any one of claims 2 to 7, or a pharmaceutically acceptable salt thereof, wherein n is 0.

10. The compound of any one of claims 1 to 9, or a pharmaceutically acceptable

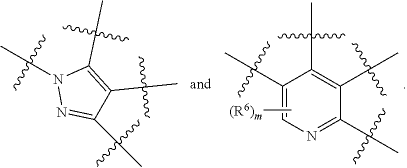

11. The compound of any one of claims 1 to 10, or a pharmaceutically acceptable salt thereof, wherein m is 0 or 1.

12. The compound of any one of claims 1 to 10, or a pharmaceutically acceptable salt thereof, wherein m is 0.

13. The compound of any one of claims 1 to 12, or a pharmaceutically acceptable salt thereof, wherein R¹ is selected from H, halo, and C₁₋₃ alkyl.

14. The compound of any one of claims 1 to 12, or a pharmaceutically acceptable salt thereof, wherein R¹ is halo.

15. The compound of any one of claims 1 to 12, or a pharmaceutically acceptable salt thereof, wherein R¹ is fluoro.

16. The compound of any one of claims 1 to 15, or a pharmaceutically acceptable salt thereof, wherein R² is selected from H, halo, and C₁₋₃ alkyl.

17. The compound of any one of claims 1 to 15, or a pharmaceutically acceptable salt thereof, wherein R² is selected from H.

18. The compound of any one of claims 1 to 17, or a pharmaceutically acceptable salt thereof, wherein R³ is selected from H, halo, and C₁₋₃ alkyl.

19. The compound of any one of claims 1 to 17, or a pharmaceutically acceptable salt thereof, wherein R³ is H.

20. The compound of any one of claims 1 to 19, or a pharmaceutically acceptable salt thereof, wherein R⁴ is selected from H, halo, and C₁₋₃ alkyl.

21. The compound of any one of claims 1 to 19, or a pharmaceutically acceptable salt thereof, wherein R⁴ is H.

22. The compound of any one of claims 1 to 17, or a pharmaceutically acceptable salt thereof, wherein R³ and R⁴ are each H.

23. The compound of any one of claims 1 to 22, or a pharmaceutically acceptable salt thereof, wherein Ring C is 4-7 membered heterocycloalkyl.

24. The compound of any one of claims 1 to 22, or a pharmaceutically acceptable salt thereof, wherein Ring C is

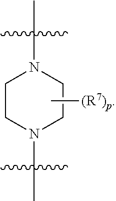

25. The compound of any one of claims 1 to 24, or a pharmaceutically acceptable salt thereof, wherein p is 0 or 1.

26. The compound of any one of claims 1 to 24, or a pharmaceutically acceptable salt thereof, wherein p is 0.

27. The compound of any one of claims 1 to 26, or a pharmaceutically acceptable salt thereof, wherein L is selected from a bond, —O—, and —N(R^{L})—.

28. The compound of any one of claims 1 to 26, or a pharmaceutically acceptable salt thereof, wherein L is a bond.

29. The compound of any one of claims 1 to 28, or a pharmaceutically acceptable salt thereof, wherein Ring D is 5-10 membered heteroaryl.

30. The compound of any one of claims 1 to 28, or a pharmaceutically acceptable salt thereof, wherein Ring D is pyridinyl.

31. The compound of any one of claims 1 to 30, or a pharmaceutically acceptable salt thereof, wherein q is 1, 2, or 3.

32. The compound of any one of claims 1 to 30, or a pharmaceutically acceptable salt thereof, wherein q is 2.

33. The compound of any one of claims 1 to 32, or a pharmaceutically acceptable salt thereof, wherein each R⁸ is independently selected from halo, C₁₋₆ alkyl, C₂₋₆ alkenyl, C₂₋₆ alkynyl, C₁₋₆ haloalkyl, —CN, —OR^{a8}, —NR^{c8}R^{d8}, —C(O)R^{a8}, —C(O)OR^{a8}, —C(O)NR^{c8}R^{d8}, and —NR^{c8}C(O)R^{a8}.

34. The compound of any one of claims 1 to 32, or a pharmaceutically acceptable salt thereof, wherein each R⁸ is independently selected from C₁₋₆ alkyl, C₁₋₆ haloalkyl, —C(O)NR^{c8}R^{d8}, and —NR^{c8}C(O)R^{a8}.

35. The compound of any one of claims 1 to 32, or a pharmaceutically acceptable salt thereof, wherein each R⁸ is independently selected from C₁₋₆ alkyl and —C(O)NR^{c8}R^{d8}; and
each R^{c8} and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, and 5-6 membered heteroaryl.

36. The compound of any one of claims 1 to 32, or a pharmaceutically acceptable salt thereof, wherein each R⁸ is independently selected from C₁₋₃ alkyl and —C(O)NR^{c8}R^{d8}; and
each R^{c8} and R^{d8} is independently selected from H, C₁₋₆ alkyl, and C₃₋₇ cycloalkyl.

37. The compound of any one of claims 1 to 32, or a pharmaceutically acceptable salt thereof, wherein each R⁸ is independently selected from methyl, methylaminocarbonyl, and cyclopropylaminocarbonyl.

38. The compound of claim 2, or a pharmaceutically acceptable salt thereof, wherein:
X is C or N;
Y is C or N;
Z is C or N; [symbol]
is a single or double bond;
n is 0, 1, 2, 3, or 4;
m is 0, 1, 2, 3, or 4;
p is 0, 1, 2, 3, or 4;
q is 0, 1, 2, 3, or 4;
L is selected from a bond, —O—, and —N(R^{L})—;
each R^{L} is independently selected from H, C₁₋₆ alkyl, and C₁₋₆ haloalkyl;
Ring A is 5-10 membered heterocycloalkyl;
Ring B is 5-6 membered heteroaryl;
Ring C is 4-10 membered heterocycloalkyl;
Ring D is 5-10 membered heteroaryl;
R¹ is selected from H, halo, and C₁₋₃ alkyl;
R² is selected from H, halo, and C₁₋₃ alkyl;
R³ is selected from H, halo, and C₁₋₃ alkyl;
R⁴ is selected from H, halo, and C₁₋₃ alkyl;
each R⁸ is independently selected from oxo, halo, C₁₋₆ alkyl, C₂₋₆ alkenyl, C₂₋₆ alkynyl, C₁₋₆ haloalkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, —C(═NR^{e8})R^{a8}, —C(═NR^{e8})NR^{c8}R^{d8}, —NR^{c8}C(═NR^{e8})R^{a8}, —NR^{c8}C(═NR^{e8})NR^{c8}R^{d8}, —NR^{c8}S(O)(═NR^{e8})R^{b8}, —NR^{c8}S(O)(═NR^{e8})NR^{c8}R^{d8}, —OS(O)(═NR^{e8})R^{b8}, —S(O)(═NR^{e8})R^{b8}, —S(O)(═NR^{e8})NR^{c8}R^{d8}, —C(O)NR^{c8}S(O)₂R^{b8}, —C(O)NR^{c8}S(O)₂NR^{c8}R^{d8}, —S(O)₂NR^{c8}C(O)R^{b8}, —NR^{c8}S(O)NR^{c8}C(O)R^{b8}, and —P(O)R^{f8}R^{g8}, wherein the C₁₋₆ alkyl, C₂₋₆ alkenyl, C₂₋₆ alkynyl, and C₁₋₆ haloalkyl of R⁸ are each optionally substituted with 1, 2, 3, 4, 5, or 6 independently selected R^{8A} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{c8}, and R^{d8} are each optionally substituted with 1, 2, 3, 4, 5, or 6 independently selected R^{8A} substituents;
or, any R^{b8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{b8} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8} are each optionally substituted with 1, 2, 3, 4, 5, or 6 independently selected R^{8A} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{c8A}R^{d8A}, —NO₂, —C(O)R^{a8A}, —C(O)OR^{a8A}, —C(O)NR^{c8A}R^{d8A}, —C(O)NR^{c8A}(OR^{a8A}), —OC(O)R^{a8A}, —OC(O)NR^{c8A}R^{d8A}, —OC(O)OR^{a8A}, —OS(O)₂R^{b8A}, —OS(O)₂NR^{c8A}R^{d8A}, —NR^{c8A}C(O)R^{a8A}, —NR^{c8A}C(O)OR^{a8A}, —NR^{c8A}C(O)NR^{c8A}R^{d8A}, —NR^{c8A}S(O)₂R^{b8A}, —NR^{c8A}S(O)₂NR^{c8A}R^{d8A}, —NR^{e8A}OR^{a8A}, —NR^{c8A}S(O)R^{b8A}, —NR^{e8A}S(O)NR^{c8A}R^{d8A}, —S(O)R^{b8A}, —S(O)₂R^{b8A}, —S(O)NR^{c8A}R^{d8A}, —S(O)₂NR^{c8A}R^{d8A}, C(═NR^{c8A})R^{b8A}, —C(═NR^{c8A})R^{c8A}R^{d8A}, —NR^{c8A}C(═NR^{c8A})R^{a8A}, —NR^{c8A}C(═NR^{c8A})NR^{c8A}R^{d8A}, —NR^{e8A}S(O)(═NR^{c8A})R^{b8A}, —NR^{e8A}S(O)(═NR^{c8A})NR^{c8A}R^{d8A}, —OS(O)(═NR^{c8A})R^{b8A}, —S(O)(═NR^{c8A})R^{b8A}, —S(O)(═NR^{c8A})R^{c8A}R^{d8A}, —C(O)NR^{e8A}S(O)₂R^{b8A}, —C(O)NR^{c8A}S(O)₂NR^{c8A}R^{d8A}, —S(O)₂NR^{c8A}C(O)R^{b8A}, —NR^{e8A}S(O)NR^{c8A}C(O)R^{b8A} and —P(O)R^{f8A}R^{g8A};
each R^{a8A}, R^{b8A}, and R^{d8A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{b8A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{c8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and
each R^{f8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl.

39. The compound of claim 2, or a pharmaceutically acceptable salt thereof, wherein:
X is C or N;
Y is C or N;
Z is C or N; [symbol]
is a single or double bond;
n is 0, 1, 2, 3, or 4;
m is 0, 1, 2, 3, or 4;
p is 0, 1, 2, 3, or 4;
q is 0, 1, 2, 3, or 4;
L is selected from a bond, —O—, and —N(R^{L})—;
each R^{L} is independently selected from H, C₁₋₆ alkyl, and C₁₋₆ haloalkyl;
Ring A is 5-10 membered heterocycloalkyl;
Ring B is 5-6 membered heteroaryl;
Ring C is 4-10 membered heterocycloalkyl;
Ring D is 5-10 membered heteroaryl;
R¹ is selected from H, halo, and C₁₋₃ alkyl;
R² is selected from H, halo, and C₁₋₃ alkyl;
R³ is selected from H, halo, and C₁₋₃ alkyl;
R⁴ is selected from H, halo, and C₁₋₃ alkyl;
each R⁸ is independently selected from halo, C₁₋₆ alkyl, C₂₋₆ alkenyl, C₂₋₆ alkynyl, C₁₋₆ haloalkyl, —CN, —OR^{a8}, —NR^{c8}R^{d8}, —C(O)R^{a8}, —C(O)OR^{a8}, —C(O)NR^{c8}R^{d8}, and —NR^{c8}C(O)R^{a8};
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl.

40. The compound of claim 1 or 2, which is a compound of Formula III: 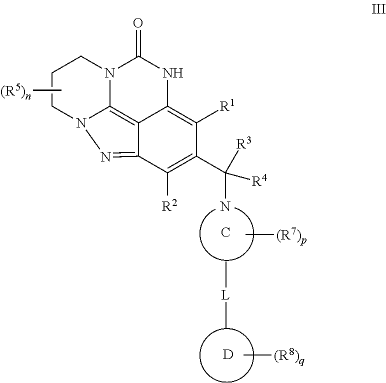
or a pharmaceutically acceptable salt thereof.

41. The compound of claim 1 or 2, which is a compound of Formula IV: 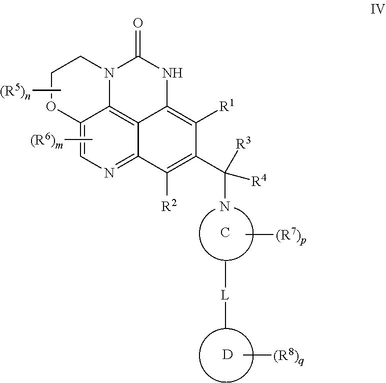
or a pharmaceutically acceptable salt thereof.

42. The compound of claim 1 or 2, which is a compound of Formula V: 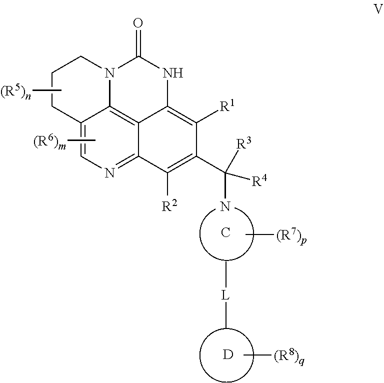
or a pharmaceutically acceptable salt thereof.

43. The compound of claim 1 or 2, wherein the compound is selected from:
5-(4-((7-fluoro-9-oxo-2,3,8,9-tetrahydro-1H-3a,4,8,9a-tetraazacyclopenta[def]phenanthren-6-yl)methyl)piperazin-1-yl)-N,6-dimethylpicolinamide;
N-cyclopropyl-5-(4-((7-fluoro-9-oxo-2,3,8,9-tetrahydro-1H-3a,4,8,9a-tetraazacyclopenta[def]phenanthren-6-yl)methyl)piperazin-1-yl)-6-methylpicolinamide;
5-(4-((6-Fluoro-4-oxo-2,3,4,5-tetrahydro-1-oxa-3a,5,9-triazapyren-7-yl)methyl)piperazin-1-yl)-N,6-dimethylpicolinamide;
N-cyclopropyl-5-(4-((6-fluoro-4-oxo-2,3,4,5-tetrahydro-1-oxa-3a,5,9-triazapyren-7-yl)methyl)piperazin-1-yl)-6-methylpicolinamide;
5-(4-((7-Fluoro-5-oxo-2,3,5,6-tetrahydro-1H-[1,6]naphthyridino[1,8,7-cde]quinazolin-8-yl)methyl)piperazin-1-yl)-N,6-dimethylpicolinamide; and
N-cyclopropyl-5-(4-((7-fluoro-5-oxo-2,3,5,6-tetrahydro-1H-[1,6]naphthyridino[1,8,7-cde]quinazolin-8-yl)methyl)piperazin-1-yl)-6-methylpicolinamide;
or a pharmaceutically acceptable salt thereof.

44. A compound, which is selected from:
5-(4-((3-ethyl-9-fluoro-2-oxo-2,3-dihydro-1H-pyrimido[4,5,6-de]quinazolin-8-yl)methyl)piperazin-1-yl)-N-(2-hydroxyethyl)-6-methylpicolinamide;
N-(2-cyanoethyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-(cyanomethyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-N-((1r,3r)-3-hydroxycyclobutyl)-6-methylpicolinamide;
5-(4-((3-ethyl-2-oxo-2,3-dihydro-1H-pyrimido[4,5,6-de]quinazolin-8-yl)methyl)piperazin-1-yl)-N-(2-hydroxyethyl)-6-methylpicolinamide;
N-(2-cyanoethyl)-5-(4-((3-ethyl-2-oxo-2,3-dihydro-1H-pyrimido[4,5,6-de]quinazolin-8-yl)methyl)piperazin-1-yl)-6-methylpicolinamide;
N-(cyanomethyl)-5-(4-((3-ethyl-2-oxo-2,3-dihydro-1H-pyrimido[4,5,6-de]quinazolin-8-yl)methyl)piperazin-1-yl)-6-methylpicolinamide;
N-cyclopropyl-5-(4-((3-ethyl-2-oxo-2,3-dihydro-1H-pyrimido[4,5,6-de]quinazolin-8-yl)methyl)piperazin-1-yl)-6-methylpicolinamide;
N-(1-cyanocyclopropyl)-5-(4-((3-ethyl-2-oxo-2,3-dihydro-1H-pyrimido[4,5,6-de]quinazolin-8-yl)methyl)piperazin-1-yl)-6-methylpicolinamide;
N-cyclobutyl-5-(4-((3-ethyl-2-oxo-2,3-dihydro-1H-pyrimido[4,5,6-de]quinazolin-8-yl)methyl)piperazin-1-yl)-6-methylpicolinamide;
5-(4-((3-ethyl-2-oxo-2,3-dihydro-1H-pyrimido[4,5,6-de]quinazolin-8-yl)methyl)piperazin-1-yl)-N-((1s,3s)-3-hydroxycyclobutyl)-6-methylpicolinamide;
5-(4-((3-ethyl-2-oxo-2,3-dihydro-1H-pyrimido[4,5,6-de]quinazolin-8-yl)methyl)piperazin-1-yl)-N-((1r,3r)-3-hydroxycyclobutyl)-6-methylpicolinamide;
N-((1R,5S,6r)-3-oxabicyclo[3.1.0]hexan-6-yl)-5-(4-((3-ethyl-2-oxo-2,3-dihydro-1H-pyrimido[4,5,6-de]quinazolin-8-yl)methyl)piperazin-1-yl)-6-methylpicolinamide;
N-((1r,3r)-3-cyanocyclobutyl)-5-(4-((3-ethyl-2-oxo-2,3-dihydro-1H-pyrimido[4,5,6-de]quinazolin-8-yl)methyl)piperazin-1-yl)-6-methylpicolinamide;
N-((1s,3s)-3-cyanocyclobutyl)-5-(4-((3-ethyl-2-oxo-2,3-dihydro-1H-pyrimido[4,5,6-de]quinazolin-8-yl)methyl)piperazin-1-yl)-6-methylpicolinamide;
N-(1-(cyanomethyl)cyclopropyl)-5-(4-((3-ethyl-2-oxo-2,3-dihydro-1H-pyrimido[4,5,6-de]quinazolin-8-yl)methyl)piperazin-1-yl)-6-methylpicolinamide;
N-cyclopropyl-5-(4-((3-cyclopropyl-2-oxo-2,3-dihydro-1H-pyrimido[4,5,6-de]quinazolin-8-yl)methyl)piperazin-1-yl)-6-methylpicolinamide; and
5-(4-((3-(cyanomethyl)-2-oxo-2,3-dihydro-1H-pyrimido[4,5,6-de]quinazolin-8-yl)methyl)piperazin-1-yl)-N-cyclopropyl-6-methylpicolinamide;
or a pharmaceutically acceptable salt thereof.

45. A pharmaceutical composition comprising a compound of any one of claims 1 to 44, or a pharmaceutically acceptable salt thereof, and a pharmaceutically acceptable carrier.

46. A method of inhibiting PARP1, comprising administering to a subject the compound of any one of claims 1 to 44, or a pharmaceutically acceptable salt thereof.

47. A method of treating a disease, disorder, or condition associated with PARP1, comprising administering to a subject in need thereof the compound of any one of claims 1 to 44, or a pharmaceutically acceptable salt thereof.

48. A method of treating cancer, comprising administering to a subject in need thereof the compound of any one of claims 1 to 44, or a pharmaceutically acceptable salt thereof.

49. The method of claim 48, wherein the cancer is selected from ovarian cancer, breast cancer, prostate cancer, and pancreatic cancer.
